(19) United States
(12) Patent Application Publication
(10) Pub. No.: US 2022/0372136 A1
(43) Pub. Date: Nov. 24, 2022

(54) METHODS FOR TREATING ANEMIA OF CHRONIC DISEASE

(71) Applicant: Disc Medicine, Inc., Watertown, MA (US)

(72) Inventors: John Quisel, Watertown, MA (US); Maria Beconi, Watertown, MA (US); Steven Robinette, Fremont, NH (US); Brian MacDonald, Newton Square, PA (US)

(73) Assignee: Disc Medicine, Inc., Watertown, MA (US)

(21) Appl. No.: 17/764,143

(22) PCT Filed: Sep. 25, 2020
(86) PCT No.: PCT/US2020/052748

§ 371 Date: Mar. 25, 2022

Related U.S. Application Data
(60) Provisional application No. 63/072,062, filed on Aug. 28, 2020.
(60) Provisional application No. 63/063,881, filed on Aug. 10, 2020.
(60) Provisional application No. 62/907,232, filed on Sep. 27, 2019.

(51) Int. Cl. C07K 16/28; A61P 7/06; A61K 45/06; A61K 39/395

(52) U.S. Cl. CPC C07K 16/28; A61P 7/06; A61K 45/06; A61K 39/3955

(57) ABSTRACT

Aspects of the application provide hepcidin antagonists and methods of using the same in treating anemias of chronic disease, iron-restricted anemias, and/or conditions associated with anemia. Accordingly, aspects of the present disclosure relate to treating a subject with high hepcidin levels (e.g., ACD) by inhibiting the BMP signaling pathway (e.g., hemojuvelin-induced BMP signaling pathway) and/or the inflammatory response (e.g., IL-6 mediated inflammatory pathway).

### RELATED APPLICATIONS

This application claims the benefit under 35 U.S.C. § 119(e) of U.S. provisional application Ser. No. 62/907,232, filed Sep. 27, 2019, entitled “HEPCIDIN ANTAGONISTS FOR TREATING ANEMIA OF CHRONIC DISEASE,” 63/063,881, filed Aug. 10, 2020, entitled “METHODS FOR TREATING ANEMIA OF CHRONIC DISEASE,” and 63/072,062, filed Aug. 28, 2020, entitled “METHODS FOR TREATING ANEMIA OF CHRONIC DISEASE,” the entire contents of each of which are incorporated herein by reference.

### BACKGROUND

Iron is a key component of oxygen-transporting storage molecules, such as hemoglobin and myoglobin. Iron deficiency results in anemia, while iron overload leads to tissue damage and fibrosis. Hepcidin is a key peptide hormonal regulator of systemic iron homeostasis. It exerts its regulatory function by binding to the cellular iron exporter ferroportin, a transmembrane protein present on hepatocytes, enterocytes in the duodenum, macrophages, and adipocytes. The binding of hepcidin promotes ferroportin degradation, preventing the export of iron from cells and release of iron into the plasma.

### SUMMARY

Aspects of the disclosure provide methods for treating high hepcidin disorders. In particular, methods are provided for treating anemia of chronic disease (ACD). ACD is a type of anemia that often arises in chronic conditions that involve inflammation. ACD can impair underlying inflammatory processes associated with chronic conditions and is a significant contributor to negative outcomes. ACD generally arises through mechanisms driven by immune and inflammatory effects that include shortened erythrocyte survival, impaired proliferation of erythroid progenitor cells, as well as abnormalities in iron metabolism. In some embodiments, effects of iron metabolism observed in ACD are associated with hepcidin upregulation. Hepcidin expression in hepatocytes mainly involves two signaling pathways. In some embodiments, hepcidin expression is regulated by the bone morphogenetic protein (BMP) signaling pathway (e.g., BMP6 induced signaling pathway). Expression of hepcidin through BMP signaling pathway is facilitated by a membrane bound co-receptor, hemojuvelin. In some embodiments, hepcidin expression is induced by increased inflammatory cytokines, causing macrophage iron sequestration and iron-restricted erythropoiesis. Moreover, in ACD, proinflammatory cytokines that induce hepcidin synthesis, such as IL-6 and oncostatin-M, are typically increased and associated with this iron sequestration, macrophage iron loading, as well as myeloid proliferation and macrophage activation (See, e.g., FIG. 1).

ACD is often associated with conditions having chronic immune activation, including, for example, chronic kidney disease, chronic renal failure, cancer (e.g., solid tumors, haematological, such as myeloma), infection (e.g., viral, bacterial, parasitic, fungal), autoimmune disease (e.g., rheumatoid arthritis, dystemic lupus erythematosus and related conditions, such as vasculitis, sarcoidosis, and inflammatory bowel disease) and other chronic conditions that involve inflammation, such as diabetes and chronic heart failure. In some embodiments, methods provided herein are useful for treating ACD associated with such conditions.

Accordingly, aspects of the present disclosure relate to treating a subject with high hepcidin levels (e.g., ACD) by inhibiting the BMP signaling pathway (e.g., hemojuvelin-induced BMP signaling pathway) and/or the inflammatory response (e.g., IL-6 mediated inflammatory pathway). Certain aspects of this disclosure relate to an observation that hemojuvelin (HJV) is a regulator of hepcidin synthesis and that loss of hemojuvelin function may be associated with iron overload. For example, in some embodiments, homozygous HJV knockdown animals fail to amplify hepcidin synthesis in response to IL-6 and are unable to mount an effective hypoferremic response to acute inflammation. Accordingly, in some embodiments, methods provided herein involve administering to a subject in need thereof a hepcidin antagonist, which may be a hemojuvelin-induced BMP signaling antagonist and/or an antagonist for IL-6 mediated signaling (e.g., JAK-STAT inhibitor), in an amount effective to treat ACD. In some embodiments, the hemojuvelin-induced BMP signaling antagonist and be combined with one or more additional therapeutic agents (e.g., JAK-STAT inhibitor, immunomodulatory agent/erythropoietin stimulating agent or erythropoietin (EPO), BET inhibitor, or GDF traps).

In some aspects, the present disclosure, at least in part, relates to a method of treating a subject having an anemia of chronic disease, the method comprising: administering to the subject an effective amount of a hepcidin antagonist. In some embodiments, the subject has impaired iron availability/functional iron deficiency.

In some embodiments, the hepcidin antagonist is a hemojuvelin-induced BMP signaling antagonist. In some embodiments, the hemojuvelin-induced BMP signaling antagonist is a BMP antagonist. In some embodiments, the BMP antagonist is a BMP2, BMP4, BMP5 or BMP6 antagonist. In some embodiments, the BMP antagonist is BMP6 antagonist. In some embodiments, the hemojuvelin-induced BMP signaling antagonist selectively inhibits its target molecule. In some embodiments, the target molecule is a BMP receptor. In some embodiments, the hemojuvelin-induced BMP signaling antagonist selectively inhibits its target molecule compared with a reference molecule. In some embodiments, the reference molecule is JAK2. In some embodiments, the hemojuvelin-induced BMP signaling antagonist selectively inhibits its target molecule compared with the reference molecule, such that it has an half maximal inhibitory concentration (IC₅₀) for the reference molecule that is at least 10-fold higher (e.g., in the range of 10¹ to 10⁶-fold higher) than the IC₅₀ for the target molecule, as measured in a kinase potency assay. In some embodiments, the hemojuvelin-induced BMP signaling antagonist is a BMP6 neutralizing antibody. In some embodiments, the BMP6 neutralizing antibody is LY311359, CSJ137, or KY1070.

In some embodiments, the Hemojuvelin-induced BMP signaling antagonist is sHJV or a soluble hemojuvelin-Fc fusion protein. In some embodiments, the soluble HJV-Fc fusion protein is FMX8.

In some embodiments, the hemojuvelin-induced BMP signaling is a modified heparin selected from: SST0001, RO-82, RO-68, NAc-91, and NacRO-00.

In some embodiments, the Hemojuvelin-induced BMP signaling antagonist is recombinant SMAD6 or SMAD7.

In some embodiments, the hepcidin antagonist is a hepcidin neutralizing agent. In some embodiments, the hepcidin neutralizing agent is NOX-94, a PEGylated L-stereoisomer RNA aptamer that binds and neutralizes hepcidin. In some embodiments, the hepcidin neutralizing agent is PRS-080, an anticalin against hepcidin. In some embodiments, the hepcidin neutralizing agent is LY2787106, a monoclonal antibody targeting hepcidin.

In some embodiments, the hemojuvelin-induced BMP signaling antagonist is an ALK2 antagonist. In some embodiments, the ALK2 antagonist is INCB000928, KER-047 or BLU-782.

In some embodiments, the hepcidin antagonist is a hemojuvelin antagonist. In some embodiments, the hemojuvelin antagonist is an anti-hemojuvelin antibody. In some embodiments, the anti-hemojuvelin antibody preferentially binds RGMc versus RGMa and RGMb. In some embodiments, the anti-hemojuvelin antibody binds RGMc with an equilibrium dissociation constant (K_{D}) less than 100 nM. In some embodiments, the anti-HJV antibody is HJV-35202. In some embodiments, the anti-HJV antibody is an anti-HJV antibody in Table 1.

In some embodiments, the anti-hemojuvelin antibody comprises: (a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or (b) a variable light chain region comprising a CDR1 comprising an amino acid sequence of SEQ ID NO: 4, a CDR2 comprising an amino acid sequence of SEQ ID NO: 5, and a CDR3 comprising an amino acid sequence of SEQ ID NO: 6.

In some embodiments, the anti-hemojuvelin antibody comprises: (a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or (b) a variable light chain region comprising a CDR1 comprising an amino acid sequence of SEQ ID NO: 7, a CDR2 comprising an amino acid sequence of SEQ ID NO: 8, and a CDR3 comprising an amino acid sequence of SEQ ID NO: 9.

In some embodiments, the anti-hemojuvelin antibody comprises: (a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or (b) a variable light chain region comprising a CDR1 comprising an amino acid sequence of SEQ ID NO: 10, a CDR2 comprising an amino acid sequence of SEQ ID NO: 11, and a CDR3 comprising an amino acid sequence of SEQ ID NO: 12.

In some embodiments, the anti-hemojuvelin antibody comprises: (a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or (b) a variable light chain region comprising a CDR1 comprising an amino acid sequence of SEQ ID NO: 13, a CDR2 comprising an amino acid sequence of SEQ ID NO: 14, and a CDR3 comprising an amino acid sequence of SEQ ID NO: 15.

In some embodiments, the anti-hemojuvelin antibody comprises: (a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or (b) a variable light chain region comprising a CDR1 comprising an amino acid sequence of SEQ ID NO: 16, a CDR2 comprising an amino acid sequence of SEQ ID NO: 17, and a CDR3 comprising an amino acid sequence of SEQ ID NO: 18.

In some embodiments, the anti-hemojuvelin antibody comprises: (a) a variable heavy chain region comprising a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21; and/or (b) a variable light chain region comprising a CDR1 comprising an amino acid sequence of SEQ ID NO: 22, a CDR2 comprising an amino acid sequence of SEQ ID NO: 23, and a CDR3 comprising an amino acid sequence of SEQ ID NO: 24.

In some embodiments, the subject is erythrocyte transfusion-dependent. In some embodiments, the subject is erythrocyte transfusion-independent. In some embodiments, a subject treated receives occasional transfusions but is not classified as transfusion dependent.

In some embodiments, the anemia of chronic disease is an iron-restricted anemia. In some embodiments, the iron-restricted anemia is associated with a functional iron deficiency. In some embodiments, the anemia of chronic disease is associated with (or caused by) chronic kidney disease.

In some embodiments, the anemia of chronic disease is associated with (or caused by) cancer. In some embodiments, the cancer is a myeloma.

In some embodiments, the anemia of chronic disease is associated with (or caused by) a chronic infection. In some embodiments, the infection is a bacterial, viral, fungal or parasitic infection.

In some embodiments, the anemia of chronic disease is associated with (or caused by) an autoimmune disease.

In some embodiments, the anemia of chronic disease is associated with (or caused by) a chronic disease that involves inflammation, such as inflammatory bowel disease, diabetes or heart failure.

In some embodiments, the subject is identified prior to the treatment as having high hepcidin levels. In some embodiments, the subject is identified as having a functional iron deficiency. In some embodiments, the subject is identified as exhibiting inflammation and/or iron-restricted erythropoiesis. In some embodiments, the subject is a human. In some embodiments, the subject does not have a nutritional iron deficiency.

In some embodiments, the subject has ferritin levels above 100 μg/L. In some embodiments, the subject has reticulocytes hemoglobin content less than 26 pg/cell. In some embodiments, the subject has a transferrin saturation level less than 50%. In some embodiments, the subject has hepatic iron levels higher than 2000 μg/g dry weight. In some embodiments, the subject has serum iron levels in a range of less than 50 μg/dL. In some embodiments, the subject has a total iron binding capacity in a range of less than 400 μg/dL. In some embodiments, the subject has hepcidin levels in a range of more than 55 ng/ml. In some embodiments, the subject has IL-6 levels of more than 1.8 pg/mL. In some embodiments, the subject has serum creatinine values of more than 2 mg/dL. In some embodiments, the subject has been identified as having hemoglobin levels in the range of 1.5 to 2.0 g/dL or 2.0 to 4.0 g/dL or more below normal hemoglobin levels. In some embodiments, the subject presents with a serum hemoglobin level of less than 10 g/dL. In some embodiments, the subject presents with a serum hemoglobin level of less than 8 g/dL. In some embodiments, the administration of the hepcidin antagonist increases hemoglobin level at least 1 g/dL from baseline.

In some embodiments, the subject presents with thrombocytopenia, anemia, and/or neutropenia. In some embodiments, the subject has received one or more transfusions. In some embodiments, the subject has transfusion-dependent anemia. In some embodiments, has received multiple transfusions over a twelve-week period.

In some embodiments, the subject has previous received one or more administrations of a JAK/STAT antagonist as a treatment for a chronic disease. In some embodiments, the subject received treatment with the JAK/STAT antagonist for 2-6 weeks. In some embodiments, the JAK/STAT antagonist is selective for JAK1 or JAK2. In some embodiments, the JAK/STAT antagonist is not active against ACVR1 (ALK2). In some embodiments, the JAK/STAT antagonist is ruxolitinib, fedratinib, pacritinib, baricitinib, tofacitinib, oclacitinib, or NSC13626. In some embodiments, the JAK/STAT antagonist inhibits IL6 mediated STAT3 activation. In some embodiments, JAK/STAT antagonist is GS-0387 or CYT-387.

In some embodiments, the method further comprising administering the subject with one or more additional therapeutic agents. In some embodiments, the additional therapeutic agent is selected from a GDF trap, a Bromodomain and extra-terminal domain (BET) inhibitor, an erythropoiesis stimulating agent, or an immunomodulatory agent. In some embodiments, the GDF trap is sotatercept, luspatercept or KER-050. In some embodiments, the BET inhibitor is CPI-0610. In some embodiments, the immunomodulatory agent is Pomalidomide. In some embodiments, the erythropoiesis stimulating agent is Erythropoietin (EPO).

In some aspects, the present disclosure also provides a method of treating a subject having an anemia of chronic disease, the method comprising administering to the subject an effective amount of a hepcidin antagonist, and one or more additional therapeutic agent. In some embodiments, the hepcidin antagonist is a HJV-induced BMP signaling antagonist, or a hepcidin neutralizing agent. In some embodiments, the HJV-induced BMP signaling antagonist is a BMP antagonist, a HJV antagonist, a modified heparin targeting BMP6, or a recombinant SMAD6 or SMAD7. In some embodiments, the BMP antagonist is a BMP6 neutralizing antibody selected from LY311359, CSJ137, and KY1070. In some embodiments, the HJV-induced BMP signaling antagonist is an HJV antagonist. In some embodiments, the HJV antagonist is an anti-HJV antibody. In some embodiments, the additional therapeutic agent is selected from a GDF trap, a JAK/STAT inhibitor, a BET inhibitor, erythropoiesis stimulating agent, or an immunomodulatory agent. In some embodiments, the additional therapeutic agent is the GDF trap. In some embodiments, the GDF trap is sotatercept, luspatercept or KER-050. In some embodiments, the JAK/STAT inhibitor is ruxolitinib, fedratinib, pacritinib, baricitinib, tofacitinib, oclacitinib, NSC13626 or momelotinib. In some embodiments, the BET inhibitor is CPI-0610. In some embodiments, the immunomodulatory agent is pomalidomide. In some embodiments, the erythropoiesis stimulating agent is EPO.

In some aspects, the present disclosure provides a method of treating a subject having or at risk of having an adverse reaction to a JAK-STAT antagonist, the method comprising: administering to the subject an effective amount of hemojuvelin-induced BMP signaling antagonist.

Certain aspects of this disclosure relate to an observation that hemojuvelin (HJV) is a regulator of hepcidin synthesis and that loss of hemojuvelin function may be associated with iron overload. For example, in some embodiments, homozygous HJV knockdown animals fail to amplify hepcidin synthesis in response to IL-6 and are unable to mount an effective hypoferremic response to acute inflammation. Accordingly, in some embodiments, methods provided herein involve administering to a subject in need thereof a hepcidin antagonist, which may be a hemojuvelin antagonist, in an amount effective to treat a high-hepcidin disorder. In some embodiments, the hemojuvelin antagonist is an anti-hemojuvelin antibody. In some embodiments, the anti-hemojuvelin antibody binds RGMc as its primary mode of action (as compared with RGMa and RGMb). Accordingly, in some embodiments, the anti-hemojuvelin antibody preferentially binds RGMc versus RGMa and/or RGMb. In some embodiments, the anti-hemojuvelin antibody binds RGMc with an equilibrium dissociation constant (K_{D}) less than one hundred nanomolar (nM) (K_{D}<100 nM). However, in some embodiments, the anti-hemojuvelin antibody binds RGMc with a similar affinity as RGMa and/or RGMb.

In some embodiments, a subject treated in accordance with the present disclosure is erythrocyte-transfusion dependent. In some embodiments, the subject treated is erythrocyte-transfusion independent. In some embodiments, the subject is identified prior to the treatment as having high hepcidin levels. In some embodiments, the subject is identified as having a functional iron deficiency. In some embodiments, the subject is identified as exhibiting inflammation and/or iron-restricted erythropoiesis. In some embodiments, the subject is a human.

In some embodiments, the subject has previously received an erythropoietin stimulating agent, a JAK-STAT inhibitor, a growth factor ligand trap, or an anti-fibrotic agent. In some embodiments, the erythropoietin stimulating agent is selected from the group consisting of danazol, prednisone, thalidomide, lenalidomide, and pomalidomide. In some embodiments, the JAK-STAT inhibitor is selected from the group consisting of ruxolitinib, momelotinib, pacritinib, INCB039110, AG490, and PpYLKTK. In some embodiments, the growth factor ligand trap is sotatercept or luspatercept. In some embodiments, the anti-fibrotic agent is PRM-151.

In some embodiments, methods of treating a subject further comprise administering to the subject one or more of an erythropoietin stimulating agent, a JAK-STAT inhibitor, a growth factor ligand trap, and an anti-fibrotic agent. In some embodiments, the erythropoietin stimulating agent is selected from the group consisting of danazol, prednisone, thalidomide, lenalidomide, and pomalidomide. In some embodiments, the JAK-STAT inhibitor is selected from the group consisting of ruxolitinib, momelotinib, pacritinib, INCB039110, AG490, and PpYLKTK. In some embodiments, the growth factor ligand trap is sotatercept or luspatercept. In some embodiments, the anti-fibrotic agent is PRM-151.

In some embodiments, an anti-hemojuvelin antibody is an affinity-matured antibody. In some embodiments, the affinity-matured antibody is derived from a mouse monoclonal antibody. In some embodiments, an anti-hemojuvelin antibody is a humanized antibody. In some embodiments, an anti-hemojuvelin antibody comprises at least three complementarity determining regions (CDRs) grafted in a heterologous framework. In some embodiments, the heterologous framework comprises a human framework region, and the at least three CDRs comprise non-human CDRs. In some embodiments, the non-human CDRs are derived from a rodent. In some embodiments, the at least three CDRs comprise variable light chain CDRs. In some embodiments, the at least three CDRs comprise three variable heavy chain CDRs and three variable light chain CDRs.

The foregoing and other aspects, implementations, acts, functionalities, features and, embodiments of the present teachings can be more fully understood from the following description in conjunction with the accompanying drawings.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 depicts a myeloproliferation cycle characteristic of certain high hepcidin disorders.

FIG. 2 depicts the hepcidin stimulatory pathway and the physiological regulation of iron homeostasis by hepcidin.

FIGS. 3A-3G illustrate the role of hepcidin in functional iron deficiency (FID) and examples of regulating hepcidin level by hepcidin antagonists. FIG. 3A depicts the mechanism of functional iron deficiency. FIG. 3B shows that functional iron deficiency is a common feature of anemia of inflammation and chronic diseases including chronic kidney disease (CKD), cancer, and cardiac failure. FIG. 3C shows that functional iron deficiency is associated with high iron level and high hepcidin level. FIG. 3D is a schematic illustration of decreasing hepcidin level to normal by using hepcidin antagonists for treatment of iron restriction diseases. FIG. 3E depicts using anti-HJV antibody as one example to inhibit the HJV induced BMP signaling pathway to reduce hepcidin to normal level. FIG. 3F shows that Matriptase-2 negatively regulates hepcidin by cleaving membrane bound HJV. FIG. 3G depicts examples of possible hepcidin antagonists for regulation of hepcidin level.

FIG. 4 shows that Activin B regulates hepcidin level through both the HJV-induced BMP signaling in response to inflammation.

FIGS. 5A-5B illustrate functional iron deficiency in chronic kidney disease (CKD). FIG. 5A shows that functional iron deficiency in CKD is a vicious cycle. FIG. 5B shows pathways of CKD and inflammation leading to anemia.

FIG. 6 is a graph showing that IL-6 induces hepcidin expression in Cynomolgus macaque, and anti-HJV antibody treatment prevents inflammation-induced (IL 6) hepcidin increase in a dose-dependent manner in Cynomolgus macaque.

### DETAILED DESCRIPTION

According to some aspects, the disclosure provides hepcidin antagonists for targeting hepcidin that are effective for inhibiting hepcidin function and/or reducing hepcidin expression, particularly for modulating iron homeostasis for the treatment of anemias of chronic disease (ACD) and/or one or more symptoms or complications thereof. Accordingly, in related aspects, the disclosure provides compositions and methods for treating ACD, which may be associated with chronic kidney disease, cancer, chronic infection, autoimmune disease, or other chronic inflammatory conditions as disclosed herein.

Further aspects of the disclosure, including a description of defined terms, are provided below.

### I. DEFINITIONS

Administering: As used herein, the terms “administering” or “administration” means to provide a complex to a subject in a manner that is physiologically and/or pharmacologically useful (e.g., to treat a condition in the subject).

Anemia of Chronic Disease: As used herein, the term “anemia of chronic disease” (ACD) refers to a haematological disorder arising in the context of an illness or condition that elicits an active immune/inflammatory response resulting in a deficiency in the ability of blood to transport oxygen. Chronic conditions (e.g., lasting 3 months or longer) can give rise to a low level of iron in the blood, despite normal or even increased levels of iron stores in macrophages and hepatocytes. In this context, inflammation may prevent the use of stored iron to product sufficient healthy red blood cells, leading to anemia. In some embodiments, ACD is the result of a deficiency in red blood cells, a deficiency in hemoglobin, and/or a deficiency in total blood volume. In some embodiments, ACD is associated with an alteration of iron metabolism and diversion of body iron (e.g., via macrophage sequestration), haemophagocytosis, reduction in erythropoiesis, and/or diminished response to erythropoietin stimulation. In some embodiments, ACD may be associated with or characterized by one or more of the following: impaired production of erythropoietin (EPO), blunted marrow erythroid response to EPO, iron-restricted erythropoiesis, and a diminished pool of EPO-responsive cells in combination with an associated chronic condition associated with inflammation. Accordingly, in some embodiments, low serum iron levels can provide diagnostic indicia of the presence of ACD in a subject when observed in the presence of an underlying chronic condition or disease. In some embodiments, ACD is an iron-restricted anemia, which may be characterized by a functional iron deficiency, which may present in a subject as a result of iron accumulation in tissue macrophages. Conditions associated with ACD include diseases which share features of immune activation. Examples of conditions associated with ACD include, without limitation, chronic kidney disease, cancer (e.g., a myeloma), infection (e.g., viral, bacterial, parasitic, or fungal), malignancies (e.g., hematological or solid tumors), autoimmune disease (e.g., rheumatoid arthritis, lupus, vasculitis, sarcoidosis), renal disease (e.g., chronic renal failure), cardiac disease, and chronic disease that involves inflammation (e.g., inflammatory bowel disease, diabetes, heart failure).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as V_{H}), and/or a light (L) chain variable region (abbreviated herein as V_{L}). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the V_{H} domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the V_{H} domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Affinity Matured Antibody: “Affinity Matured Antibody” is used herein to refer to an antibody with one or more alterations in one or more CDRs, which result in an improvement in the affinity (i.e. KD, kd or ka) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies are known in the art, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); and Hawkins et al, J. Mol. Biol., 226: 889-896 (1992). Selective mutation at selective mutagenesis positions and at contact or hypermutation positions with an activity-enhancing amino acid residue is described in U.S. Pat. No. 6,914,128 B1.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

CDR: As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence) with a number, and numbers corresponding to the heavy chain and light chain CDRs are provided in Table 3. One skilled in the art is able to derive the CDR sequences of the anti-HJV antibodies provided in Table 2 using the different numbering systems as set forth in Table 3.

TABLE 3
CDR Definitions

| | IMGT¹ | Kabat² | Chothia³ |
|---|---|---|---|
| HC CDR1 | 27-38 | 31-35 | 26-32 |
| HC CDR2 | 56-65 | 50-65 | 53-55 |
| HC CDR3 | 105-116/117 | 95-102 | 96-101 |
| LC CDR1 | 27-38 | 24-34 | 26-32 |
| LC CDR2 | 56-65 | 50-56 | 50-52 |
| LC CDR3 | 105-116/117 | 89-97 | 91-96 |

¹IMGT ®, the international ImMunoGeneTics information system ®, imgt.org, Lefranc, M.-P. et al., Nucleic Acids Res., 27:209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196:901-917 (1987)

CDR-grafted antibody: The term “CDR-grafted antibody” refers to antibodies which comprise heavy and light chain variable region sequences from one species but in which the sequences of one or more of the CDR regions of V_{H} and/or V_{L} are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human hemojuvelin and non-human primate hemojuvelin) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Effective Amount: As used herein, “an effective amount” refers to the amount of each active agent (e.g., anti-HJV antibody) required to confer therapeutic effect on the subject, either alone or in combination with one or more other active agents. In some embodiments, the therapeutic effect is reduced hepcidin level or activity, increased level of transferrin saturation (TSAT %), and/or alleviated disease conditions (e.g., reduced anemia).

Hemojuvelin (HJV): As used herein, the term “hemojuvelin (HJV)” (also known as repulsive guidance molecule C (RGMc) or hemochromatosis type 2 protein (HFE2)) refers to a membrane-bound and soluble form protein that regulates hepcidin production through the BMP/SMAD signaling pathway. The HFE2 gene encodes two known classes of GPI-anchored and glycosylated HJV molecules, which are targeted to the membrane and undergo distinct fates. HJV exists in multiple isoforms, including two soluble isoforms and two membrane-associated isoforms. In some embodiments, a predominant membrane-associated isoform is a disulfide-linked two-chain form composed of N- and C-terminal fragments. In some embodiments, a full-length single-chain isoform associates with the membrane, but is released from the cell surface and accumulates in extracellular fluid. In some embodiments, HJV may be of human (NCBI Gene ID 148738), non-human primate (e.g., NCBI Gene ID 698805), or rodent (e.g., NCBI Gene ID 69585 or NCBI Gene ID 310681) origin. In addition to HJV (RGMc), the repulsive guidance molecule family includes repulsive guidance molecule A (RGMa) and repulsive guidance molecule B (RGMb). RGMa and RGMb are expressed in the central nervous system during development and are thought to be involved in controlling axonal patterning and neuronal survival, while HJV is produced in the liver and in cardiac and skeletal muscle.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Hepcidin Antagonist: As used herein, a “hepcidin antagonist” refers to an agent that reduces hepcidin expression and/or hepcidin activity (directly or indirectly). In some embodiments, a hepcidin antagonist inhibits hepcidin-induced ferroportin degradation. Accordingly, in some embodiments, a hepcidin antagonist targets hepcidin function indirectly through the hepcidin stimulatory pathway to decrease hepcidin expression. In some embodiments, a hepcidin antagonist targets hepcidin function directly, e.g., by binding the hepcidin peptide to sequester free hepcidin or by binding ferroportin to inhibit the hepcidin-ferroportin binding interaction, thereby decreasing hepcidin-induced ferroportin degradation. In some embodiments, a hepcidin antagonist is a ferroportin inhibitor that disrupts ferroportin-hepcidin interactions, such as, for example, as disclosed in Ross S L, et al., Identification of Antibody and Small Molecule Antagonists of Ferroportin-Hepcidin Interaction. Front Pharmacol. 2017 Nov. 21; 8:838; Fung E., et al., High-Throughput Screening of Small Molecules Identifies Hepcidin Antagonists. Molecular Pharmacology March 2013, 83 (3) 681-690; and Angeliki Katsarou and Kostas Pantopoulos, Hepcidin Therapeutics. Pharmaceuticals (Basel). 2018 December; 11(4): 127, the relevant contents of each of which are incorporated herein by reference.

HJV-induced BMP signaling: As used herein, the term “HJV-induced BMP signaling” refers to signaling through BMP receptors that is induced by Hemojuvelin (HJV), which is a membrane bound co-receptor for bone morphogenetic protein (BMP) signaling. As discussed in Xia Y, et al., Hemojuvelin regulates hepcidin expression via a selective subset of BMP ligands and receptors independently of neogenin, Blood. 2008 May 15; 111(10): 5195-5204, in hepatocytes, HJV-induced BMP signaling positively regulates hepcidin mRNA expression. In some embodiments, HJV binds to BMP2, BMP4, BMP5, or BMP6 to induce BMP signaling, e.g., to positively regulate hepcidin levels in hepatocytes. In some embodiments, cleavage of HJV by matripatase-2 reduces the amount of cell surface HJV available to participate in BMP signaling. In some embodiments, induction of BMP signaling by HJV is independent of neogenin. However, in some embodiments, neogenin facilitates induction of BMP signaling by HJV, as discussed in Zhao et al, Neogenin Facilitates the Induction of Hepcidin Expression by Hemojuvelin in the Liver, J Biol Chem. 2016 Jun. 3; 291(23): 12322-12335. In some embodiments, BMP6 is responsible for iron-dependent activation of the Smad signaling. In some embodiments, BMP6 is secreted from liver sinusoidal endothelial cells and binds to a BMP receptor (BMPR) on hepatocytes and thereby activates the SMAD signaling cascade. In such embodiments, HJV serves as a co-receptor for such BMP6, e.g., to positively regulate hepcidin levels in hepatocytes. In some embodiments, BMPs transduce signals by binding to one or a combination of type I and II serine/threonine kinase receptors. BMP type II receptors include BMPRII, ActRIIA, and ActRIIB BMP type I receptors include ALK3, ALK6, and ALK2. In some embodiments, upon ligand binding, constitutively active type II receptors phosphorylate type I receptors, and type I receptors then phosphorylate intracellular receptor-activated Smads (R-Smads), namely Smad 1, Smad 5 and/or Smad 8. In such embodiments, activated R-Smads complex with the common partner Smad4 and translocate to the nucleus to regulate gene transcription, e.g., induction of hepcidin expression.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences (e.g., CDRs grafted in a heterologous framework).

Humanized antibody: The term “humanized antibody” refers to antibodies which comprise heavy and light chain variable region sequences from a non-human species (e.g., a mouse) but in which at least a portion of the V_{H} and/or V_{L} sequence has been altered to be more “human-like”, i.e., more similar to human germline variable sequences. One type of humanized antibody is a CDR-grafted antibody, in which human CDR sequences are introduced into non-human V_{H} and V_{L} sequences to replace the corresponding nonhuman CDR sequences. In one embodiment, humanized anti-hemojuvelin antibodies and antigen binding portions are provided. Such antibodies may be generated by obtaining murine anti-hemojuvelin monoclonal antibodies using traditional hybridoma technology followed by humanization using in vitro genetic engineering, such as those disclosed in Kasaian et al PCT publication No. WO 2005/123126 A2.

Inhibitory nucleic acid: An inhibitory nucleic acid, as used herein, refers to nucleic acids capable of reducing expression and/or function of the target gene. Non-limiting examples of an inhibitory RNA include microRNA (miRNA), a small interfering RNA (siRNA), a short hairpin RNA (shRNA), an artificial miRNA (AmiRNA), gapmers, mixmers, or an antagomir. Inhibitory nucleic acids are useful for translational repression and/or gene silencing, e.g., via the ribonuclease mediated degradation. Inhibitor nucleic acids may be delivered directly as an oligonucleotides (e.g., isolated single stranded or double stranded oligonucleotides) and formulations thereof. In some embodiments, nucleic acids may be delivered in formulations or as conjugates that facilitate cellular uptake, e.g., GalNac conjugates. However, in some embodiments, the inhibitory nucleic acid can be delivered by a viral vector, such as a lentivirus, retrovirus, or recombinant adeno-associated virus (rAAV), which is engineered to express the inhibitory nucleic acid.

Isolated antibody: An “isolated antibody”, as used herein, is intended to refer to an antibody that is substantially free of other antibodies having different antigenic specificities (e.g., an isolated antibody that specifically binds hemojuvelin is substantially free of antibodies that specifically bind antigens other than hemojuvelin). An isolated antibody that specifically binds hemojuvelin may, however, have cross-reactivity to other antigens, such as other repulsive guidance molecule (RGM) proteins (e.g., RGMa and/or RGMb). Moreover, an isolated antibody may be substantially free of other cellular material and/or chemicals.

JAK-STAT signaling: As used herein, the term “JAK-STAT signaling” refers to signaling through cellular receptors that recruits a Janus Kinase (JAK), such as, for example, Janus Kinase 1 (JAK1) or Janus Kinase 2 (JAK2), to activate a transcription factor signal transducer and activator of transcription (STAT), such as, for example, STAT3. In some embodiments, as discussed in Maliken, B D, et al., The Hepcidin Circuits Act: Balancing Iron and Inflammation, Hepatology. 2011 May; 53(5): 1764-1766, JAK-STAT signaling involves binding of the cytokine interleukin-6 (IL-6) to its cognate cellular receptor, which then recruits Janus Kinase 2 (JAK2) to phosphorylate STAT3. In some embodiments, STAT3 is then (following JAK2 activation/phosphorylation) translocated into the nucleus. In some embodiments, activated STAT3 then induces hepcidin transcription, e.g., by binding to the STAT3 binding motif in the hepcidin promoter region. Thus, in some embodiments, hepcidin expression is induced via JAK-STAT signaling during inflammation through activation STAT3 by IL-6.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise one or more modified internucleotide linkage. In some embodiments, an oligonucleotide may comprise one or more phosphorothioate linkages, which may be in the Rp or Sp stereochemical conformation.

Recombinant Adeno-Associated virus (rAAV): The term “Recombinant adeno-associated virus (rAAV)” refers to an AAV that has been artificially produced or obtained using recombinant methods. Recombinant AAVs (rAAVs) preferably have tissue-specific targeting capabilities, such that a transgene of the rAAV will be delivered specifically to one or more predetermined tissue(s) (e.g., ocular tissues). An rAAV typically comprises an AAV capsid protein encapsulating a recombinant AAV vector. “Recombinant AAV (rAAV) vectors” are typically composed of, at a minimum, a transgene and its regulatory sequences, and 5′ and 3′ AAV inverted terminal repeats (ITRs). The AAV capsid is an important element in determining these tissue-specific targeting capabilities (e.g., tissue tropism). In some embodiments, an rAAV having a capsid appropriate for the tissue being targeted may be used. In some embodiments, the rAAV comprises an AAV capsid protein specific for liver delivery. In some embodiments, the AAV capsid protein is of an AAV2, AAV3B, AAV8, or LK03 serotype. In some embodiments, the rAAV vector comprises a liver-specific promoter driving the expression of the inhibitory nucleic acid targeting BMP-6. Non-limiting examples of the liver-specific promoter is human serum albumin promoter, alpha-1-antitrypsin promoter, Apolipoprotein E/C-I hepatic control region/human alpha-1-antitrypsin chimeric promoter, or alpha 1 microglobulin/bikunin enhancer/human thyroxine-binding globulin (TBG) chimeric promoter. AAV capsid proteins for liver specificity and liver specific promoters have been described in the art, e.g., Kattenhorn et al, Adeno-Associated Virus Gene Therapy for Liver, Human Gene Therapy, Vol. 27, No. 12.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. One embodiment of the disclosure provides fully human antibodies capable of binding human hemojuvelin which can be generated using techniques well known in the art, such as, but not limited to, using human Ig phage libraries such as those disclosed in Jermutus et al., PCT publication No. WO 2005/007699 A2.

Selective: As used herein, the term “selective” or “selectively” refers to the ability of a molecule to produce an effect in relation to its target molecule compared to a reference molecule. For example, a molecule that selectively inhibits its target molecule means that this molecule is capable of inhibiting its target molecule with a degree that is distinguishable from a reference molecule in an inhibition assay or other inhibitory context. For example, with respect to an inhibitor, the term, “selectively inhibits”, refers to the ability of the inhibitor to inhibit its target molecule with a degree that is distinguishable from a reference molecule that is not substantially inhibited in an inhibition assay, e.g., to an extent that permit selective inhibition of the target molecule, as described herein. For example, the half maximal inhibitory concentration (IC50) for the target molecule and/or the reference molecule can be tested in a kinase potency assay as described in Asshoff, M. et al., Momelotinib inhibits ACVR1/ALK2, decreases hepcidin production, and ameliorates anemia of chronic disease in rodents. Blood. 2017 Mar. 30; 129(13): 1823-1830 (e.g., Kinase potency assay by Carna Biosciences). In this assay, inhibitor solution (e.g., solution containing the selective inhibitor to be tested)/kinase substrate is mixed with target molecule solution (e.g., ALK2) or reference molecule solution (e.g., JAK1 or JAK2), and incubated under room temperature for 1 hour. Once the reaction is terminated, the signal produced by enzymatic activity on the substrate can be measured. The half maximal inhibitor concentration for the target molecule and the reference molecule can be calculated. In some embodiments, a molecule described herein selectively binds to a target molecule. In some embodiments, a molecule described herein selectively inhibits to a target molecule. In some embodiments, a molecule described herein selectively antagonizes to a target molecule. In some embodiments, a molecule described herein selectively neutralizes to a target molecule.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to hemojuvelin.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having ACD and/or one or more conditions which are associated with, or may give rise to, ACD and/or a functional iron deficiency.

Treatment: As used herein, the term “treating” or “treatment” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying or preventing the development or progression of the disease, or reducing disease severity.

### II. HEPCIDIN ANTAGONISTS

Among other aspects, the disclosure relates to hepcidin antagonists and related methods for treatment.

FIG. 2 depicts the hepcidin stimulatory pathway and the physiological regulation of iron homeostasis by hepcidin. As generically shown, Hepcidin operates by binding to the iron exporter Ferroportin in iron-releasing target cells (e.g., hepatocytes, duodenal enterocytes, tissue macrophages, and other cell types). The binding of hepcidin blocks iron efflux and triggers ubiquitination, internalization, and lysosomal degradation of ferroportin. This leads to intracellular iron retention and eventually decreased systemic iron levels. Accordingly, in some embodiments, a hepcidin antagonist of the present disclosure is a hepcidin inhibitor, which antagonizes hepcidin function by sequestering hepcidin or stabilizing ferroportin to inhibit the binding of hepcidin to ferroportin.

The HAMP gene encodes hepcidin precursor protein, which is primarily expressed by hepatocytes in the liver, and at lower levels by other cells in extrahepatic tissues. The precursor protein is subsequently cleaved to yield bioactive hepcidin. In some embodiments, a hepcidin antagonist of the present disclosure is a HAMP antagonist, which antagonizes hepcidin function by binding HAMP or a transcription or translation product thereof, or by inhibiting a transcriptional or translational regulator of HAMP to reduce HAMP expression. Examples of transcriptional regulators of HAMP include, without limitation, SMAD1/5/8 (e.g., BMP-SMAD signaling pathway) and STATS (e.g., JAK-STAT signaling pathway). Accordingly, in some embodiments, the HAMP antagonist is a BMP-SMAD signaling pathway inhibitor or a JAK-STAT signaling pathway inhibitor.

Increases in serum or tissue iron trigger transcriptional induction of hepcidin via the BMP-SMAD signaling pathway. The mechanism involves secretion of bone morphogenetic protein 6 (BMP6) from liver sinusoidal endothelial cells, which binds to a BMP receptor (BMPR) on hepatocytes and thereby activates the SMAD signaling cascade. Examples of BMP receptors include type I (e.g., ALK2, ALK3, ALK6) and type II (e.g., ActRIIA, BMPRII) BMP receptors. A central regulator of the BMP-SMAD signaling pathway for hepcidin stimulation is the BMP co-receptor, hemojuvelin (HJV). Accordingly, in some aspects, the disclosure relates to hemojuvelin antagonists for targeting hepcidin, e.g., for modulating iron homeostasis for the treatment of ACD and/or one or more conditions arising as a result of ACD. In some embodiments, the disclosure provides methods for modulating iron homeostasis for the treatment of ACD that involve direct inhibition of binding of hepcidin to the iron exporter ferroportin.

(i) Hemojuvelin-Induced BMP Signaling Antagonists

In some aspects, hemojuvelin-induced BMP signaling antagonists are provided herein to inhibit BMP-SMAD signaling for reducing expression and/or function of hepcidin, e.g., for modulating iron homeostasis for the treatment of anemia arising as a result of chronic diseases (e.g., chronic kidney diseases, cancer, infection, autoimmune disease, or inflammation). In some embodiments, such methods are based on a recognition that increases in serum or tissue iron trigger transcriptional induction of hepcidin via the BMP-SMAD signaling pathway. In some embodiments, HJV serves as a BMP co-receptor to positively regulate hepcidin levels. In certain cells, e.g., hepatocytes, HJV-induced BMP signaling positively regulates hepcidin mRNA expression. In such embodiments, HJV binds to BMP2, BMP4, BMP5, and/or BMP6 to mediate BMP signaling, e.g., to positively regulate hepcidin levels in hepatocytes. In some embodiments, BMPs transduce signals by binding to one or a combination of type I and II serine/threonine kinase receptors. In some embodiments, upon ligand binding, constitutively active type II receptors phosphorylate type I receptors, and type I receptors then phosphorylate intracellular receptor-activated Smads (R-Smads), namely Smad 1, Smad 5 and/or Smad 8. In such embodiments, activated R-Smads complex with the common partner Smad4 and translocate to the nucleus to regulate gene transcription, e.g., induction of hepcidin expression.

In some embodiments, methods provided herein utilize HJV-induced BMP signaling antagonist for the treatment of anemia associated with chronic diseases (ACD). In some embodiments, HJV-induced BMP signaling antagonist is a BMP antagonist, which directly or indirectly inhibits BMP signaling (e.g., BMP antibodies, inhibitory nucleic acid for BMPs, soluble BMP receptors, soluble hemojuvelin, etc.). In some embodiments, the BMP antagonist is an anti-BMP antibody that inhibits signaling. In some embodiment, recombinant noggin is provided as a BMP antagonist. In some embodiments, an anti-BMP antibody specifically binds to and inhibits a particular BMP, e.g., BMP6. However, in some embodiments, anti-BMP binds to and inhibits multiple BMPs. In some embodiments, the anti-BMP antibody is an antibody against the BMPs that binds to HJV.

In some embodiments, the anti-BMP antibody is an anti-BMP2 antibody that specifically binds to BMP2 and inhibits downstream signaling. Suitable anti-BMP2 antibodies are disclosed, for example, in Gorrell R E, et al., Identification of a bone morphogenetic protein type 2 receptor neutralizing antibody. BMC Res Notes. 2019; 12: 331.; and Kang M H, et al., BMP2 accelerates the motility and invasiveness of gastric cancer cells via activation of the phosphatidylinositol 3-kinase (PI3K)/Akt pathway. Exp Cell Res. 2010 Jan. 1; 316(1):24-37, the contents of each of which are incorporated herein by reference.

In some embodiments, the anti-BMP antibody is an anti-BMP4 antibody that specifically binds BMP4 and inhibits downstream signaling. Suitable anti-BMP4 antibodies are disclosed, for example, in Calpe S. et al., Comparison of newly developed anti-bone morphogenetic protein 4 llama-derived antibodies with commercially available BMP4 inhibitors. MAbs. 2016 May-June; 8(4): 678-688, the contents of which are incorporated herein by reference.

In some embodiments, the BMP-2 and/or BMP4 antagonists are BMP2 and/or BMP4 antagonist as disclosed in U.S. Pat. No. 8,338,377, entitled “BMP-ALK3 antagonists and uses for promoting bone growth,” issued Dec. 25, 2012; U.S. Pat. No. 9,738,636, entitled “Fused heterocyclic compounds as selective BMP inhibitors,” issued Aug. 22, 2017; US2019218214, entitled “Inhibition of BMP Signaling Compounds, Compositions and Uses Thereof,” published May 21, 2019; US2019284183, entitled “Inhibition of bmp signaling, compounds, compositions and uses thereof,” published Sep. 19, 2019; US2020054643, entitled “Fused heterocyclic compounds as selective bmp inhibitors,” published Feb. 20, 2020, the contents of each of which are incorporated herein by reference.

In some embodiments, the anti-BMP antibody is an anti-BMP5 antibody that specifically binds BMP5 and inhibits downstream signaling. In some embodiments, the anti-BMP5 antibody is Human BMP-5 Antibody AF615 (R&D Systems) or Human BMP-5 Antibody MAB7151(R&D Systems), for example.

In some embodiments, the anti-BMP antibody is an anti-BMP6 antibody that specifically binds BMP6 and inhibits downstream signaling. In some embodiments, the anti-BMP6 antibody for use in the methods provided herein is an antiBMP-6 antibody as disclosed in U.S. Pat. No. 8,795,665B2, entitled “BMP-6 antibodies”, issued Aug. 5, 2014; U.S. Pat. No. 8,980,582B2, entitled “BMP-6 antibodies and DNA encoding the same,” issued Mar. 17, 2015; U.S. Pat. No. 9,439,963B2, entitled “Methods of treating anaemia”, issued Sep. 13, 2016; U.S. Pat. No. 9,862,764B2, entitled “Compositions and methods for antibodies targeting BMP6”, issued Jan. 19, 2018; WO2017216724A1, entitled “Methods for treating disease using inhibitors of bone morphogenetic protein 6 (bmp6),” published Dec. 21, 2017; and WO2017191437A1, entitled “Methods, regimens, combinations & antagonists,” published Nov. 9, 2017, WO2020065252, entitled “Antagonists”, published Apr. 2, 2020, the contents of each of which are incorporated herein by reference in their entireties. In some embodiments, the anti-BMP6 antibody is LY3113593. In some embodiments, the anti-BMP6 antibody is CSJ137. In some embodiments, the anti-BMP6 antibody is KY1070.

In some embodiments, the BMP antagonist is an inhibitory nucleic acid that inhibits expression of BMPs (e.g., dsRNA, siRNA, miRNA, shRNA, AmiRNA, antisense oligonucleotides (ASO) or aptamer targeting BMP2, BMP4, BMP5, or BMP6). In some embodiments, an inhibitory nucleic acid targeting the BMPs can be used herein in treating ACD. In some embodiments, the inhibitory nucleic acids targeting BMP6 is an inhibitory nucleic acid targeting BMP6 as disclosed, for example, in U.S. Pat. No. 9,228,188, entitled “Compositions and method for inhibiting hepcidin antimicrobial peptide (HAMP) or HAMP-related gene expression,” issued Jan. 5, 2016, the entire contents of which is incorporated herein by reference. In some embodiments, the inhibitory nucleic acid targeting BMP-6 is an inhibitory nucleic acid. In some embodiments, the inhibitory nucleic acid is an miRNA targeting BMP-6. In some embodiments, the inhibitory nucleic acid is a shRNA targeting BMP-6. In some embodiments, the inhibitory nucleic acid is a siRNA targeting BMP-6. In some embodiments, the inhibitory nucleic acid is a AmiRNA targeting BMP-6.

In some embodiments, additional examples of BMP6 antagonists include, without limitation, TP-0184, FKBP12, a twisted gastrulation protein, dorsomorphin, noggin, chordin, ventroptin, follistatin, follistatin-related gene (FLRG), heparin (e.g., SST0001, RO-82, RO-68, NAc-91, and NacRO-00), sulphated glycosaminoglycan, and Sclerostin domain-containing 1 protein (SOSTDC1). Additional examples of BMP6 antagonists that may be useful in certain methods provided herein are provided. In some embodiments, the BMP6 antagonist is a BMP6 antagonist as disclosed in in U.S. Pat. Nos. U.S. Pat. No. 8,318,167, entitled “METHODS AND COMPOSITIONS FOR REGULATING IRON HOMEOSTASIS BY MODULATION OF BMP-6,” issued Nov. 27, 2012; U.S. Pat. No. 9,556,251, entitled “METHODS AND COMPOSITIONS TO REGULATE HEPCIDIN EXPRESSION,”, issued Jan. 31, 2017; U.S. Pat. No. 9,862,764, entitled “COMPOSITIONS AND METHODS FOR ANTIBODIES TARGETING BMP6,” issued Jan. 9, 2018; U.S. Pat. No. 9,682,983, entitled “BMP INHIBITORS AND METHODS OF USE THEREOF,” issued Jun. 20, 2017; U.S. Pat. No. 8,507,501, entitled “INHIBITORS OF THE BMP SIGNALING PATHWAY,” issued Aug. 13, 2013; U.S. Pat. No. 9,738,636, entitled “FUSED HETEROCYCLIC COMPOUNDS AS SELECTIVE BMP INHIBITORS,” issued Aug. 22, 2017; and U.S. Pat. No. 8,795,665, entitled “BMP-6 ANTIBODIES,” issued Aug. 5, 2014; U.S. Publication Nos. US2010/0093760, entitled “METHODS FOR IDENTIFYING COMPOUNDS THAT MODULATE CELL SIGNALING AND METHODS EMPLOYING SUCH COMPOUND,” published Apr. 15, 2010; US2014/0199314, entitled “METHODS AND COMPOSITIONS FOR REGULATING IRON HOMEOSTASIS BY MODULATION OF BMP-6,” published Jul. 17, 2014; US2014/0086919, entitled “METHODS AND COMPOSITIONS FOR REGULATING IRON HOMEOSTASIS BY MODULATION OF BMP-6,” published Mar. 27, 2014; US2016/0263117, entitled “COMPOSITIONS AND METHODS FOR CARDIOVASCULAR DISEASE,” published Sep. 15, 2016; US2016/0115167, entitled “BMP INHIBITORS AND METHODS OF USE THEREOF,” published Apr. 28, 2016; US2017/0197968, entitled, “COMPOSITIONS AND METHODS FOR INHIBITING BMP,” published Jul. 13, 2017; US2017/0190705, entitled “COMPOSITIONS AND METHODS FOR INHIBITING BMP,” published Jul. 6, 2017; US2017/0305883, entitled “COMPOSITIONS AND METHODS FOR INHIBITING BMP,” published Oct. 26, 2017; US2018/0021340, entitled “METHODS AND COMPOSITIONS FOR THE TREATMENT OR PREVENTION OF ABNORMAL BONE FORMATION IN A SOFT TISSUE,” published Jan. 25, 2018; PCT Publication Nos. WO 2017/216724, entitled “METHODS FOR TREATING DISEASE USING INHIBITORS OF BONE MORPHOGENETIC PROTEIN 6 (BMP6), published Dec. 21, 2017; WO 2018/136634, entitled “FUSED HETEROCYCLIC COMPOUNDS AS SELECTIVE BMP INHIBITORS,” published Jul. 26, 2018; WO 2018/053234, entitled “TWISTED GASTRULATION POLYPEPTIDES AND USES THEREOF,” published Mar. 22, 2018; WO 2018/185341, entitled “REGULATOR OF BMP-SMAD SIGNALING AND USES THEREOF,” published Oct. 11, 2018; WO 2016/146651, entitled “MACROCYCLIC ACTIVIN-LIKE RECEPTOR KINASE INHIBITORS” published Sep. 22, 2016, the entire contents of each of which are incorporated herein by reference.

In some embodiments, a hemojuvelin-induced BMP signaling antagonist is a BMP receptor antagonist. In some embodiments, the BMP receptor antagonist is a neutralizing antibody against a BMP receptor. In some embodiments, BMPs transduce signals by binding to one or a combination of type I and II serine/threonine kinase receptors. BMP type II receptors include BMPRII, ActRIIA, and ActRIIB BMP type I receptors include ALK3, ALK6, and ALK2. In some embodiments, the BMP receptor antagonists are neutralizing antibodies targeting BMP receptors. In some embodiments, the BMP receptor neutralizing antibody is an anti-BMPRII antibody, an anti-ActRIIA antibody, an anti-ActRIIB antibody, an anti-ALK3 antibody, an anti-ALK6 antibody, or an anti-ALK2 antibody. In some embodiments, the BMP receptor neutralizing antibody is an anti-ALK2 antibody. In some embodiments, the anti-ALK2 antibody is an anti-ALK2 antibody as disclosed in U.S. Pat. No. 10,428,148B2, entitled “Anti-ALK2 antibody,” issued Oct. 1, 2019; WO2020086730A1, entitled “Alk2 antibodies and methods of use thereof”, published Apr. 30, 2020; US2018/0118835, entitled “ANTI-ALK2 ANTIBODY,” published May 3, 2018, the contents of each of which are incorporated herein by reference.

In some embodiments, the BMP receptor antagonist is an inhibitory nucleic acid that inhibits expression of a BMP receptor (e.g., a BMP type I receptor or BMP type II receptor). In some embodiments, the inhibitory nucleic acid is an inhibitory nucleic acid that inhibits ALK2 expression. Thus, in some embodiments, an inhibitory nucleic acid that inhibits expression of a BMP receptor can be used herein in treating ACD.

In some embodiments, the hemojuvelin-induced BMP signaling antagonist selectively inhibits its target molecule. In some embodiments, the target molecule is a BMP receptor. In some embodiments, the hemojuvelin-induced BMP signaling antagonist selectively inhibits its target molecule compared with a reference molecule. In some embodiments, the reference molecule is JAK2. In some embodiments, the target molecule is ALK2. In some embodiments, the hemojuvelin-induced BMP signaling antagonist selectively inhibits its target molecule compared with the reference molecule, such that it has an half maximal inhibitory concentration (IC50) for the reference molecule that is at least 10-fold (e.g., at least 10-fold, at least 20-fold, at least 30-fold, at least 40-fold, at least 50-fold, at least 60-fold, at least 70-fold, at least 80-fold, at least 90-fold, or higher), 10² fold (e.g., at least 100-fold, at least 200-fold, at least 300-fold, at least 400-fold, at least 500-fold, at least 600-fold, at least 700-fold, at least 800-fold, at least 900-fold, or higher), 10³ fold (e.g., at least 1000-fold, 2 at least 000-fold, at least 3000-fold, at least 4000-fold, at least 5000-fold, at least 6000-fold, at least 7000-fold, at least 8000-fold, at least 9000-fold, or higher), 10⁴ fold (e.g., at least 1×10⁴-fold, at least 2×10⁴-fold, at least 3×10⁴-fold, at least 4×10⁴-fold, at least 5×10⁴-fold, at least 6×10⁴-fold, at least 7×10⁴-fold, at least 8×10⁴-fold, at least 9×10⁴-fold, or higher), 10⁵ fold (e.g., at least 1×10⁵-fold, at least 2×10⁵-fold, at least 3×10⁵-fold, at least 4×10⁵-fold, at least 5×10⁵-fold, at least 6×10⁵-fold, at least 7×10⁵-fold, at least 8×10⁵-fold, at least 9×10⁵-fold, or higher), 10⁶ fold (e.g., at least 1×10⁶-fold, at least 2×10⁶-fold, at least 3×10⁶-fold, at least 4×10⁶-fold, at least 5×10⁶-fold, at least 6×10⁶-fold, at least 7×10⁶-fold, at least 8×10⁶-fold, at least 9×10⁶-fold, or higher), or higher compared with the target molecule. In some embodiments, the hemojuvelin-induced BMP signaling antagonist selectively inhibits its target molecule compared with the reference molecule, such that it has an half maximal inhibitory concentration (IC50) for the reference molecule is in the range of 10-fold to 10² fold, 10-fold to 10³ fold, or 10-fold to 10⁴ fold, 50-fold to 10⁵ fold or 100-fold to 10⁶ fold or higher compared with the target molecule. In some embodiments, the IC50 is determined according to a kinase potency assay (e.g., the assay described in Asshoff, M. et al., Momelotinib inhibits ACVR1/ALK2, decreases hepcidin production, and ameliorates anemia of chronic disease in rodents. Blood. 2017 Mar. 30; 129(13): 1823-1830 (e.g., Kinase potency assay by Carna Biosciences). In some embodiments, the selective BMP receptor inhibitor is a selective ALK2 inhibitor as determined by the kinase potency assay. In some embodiments, the selective BMP receptor inhibitor does not inhibit JAK1/JAK2. In some embodiments, the selective ALK2 inhibitor is not momelotinib.

In some embodiments, the BMP receptor antagonist is a small molecule inhibitor of a BMP receptor. In some embodiments, the BMP receptor antagonist is a small molecule ALK2 inhibitor. In some embodiments, an ALK2 inhibitor is an ALK2 inhibitor as disclosed in U.S. Pat. No. 10,233,186, “Inhibitors of activin receptor-like kinase,” issued on Mar. 19, 2019; U.S. Pat. No. 10,202,356, entitled “JAK2 AND ALK2 INHIBITORS AND METHODS FOR THEIR USE,” issued Feb. 12, 2019; U.S. Ser. No. 10/669,277B2, entitled “Inhibitors of activin receptor-like kinase”, issued Jun. 2, 2020, WO2019079649, entitled “Substituted pyrrolopyridines as inhibitors of activin receptor-like kinase”, published Apr. 25, 2019; WO 2018/200855, entitled “NOVEL ALK2 INHIBITORS AND METHODS FOR INHIBITING BMP SIGNALING” published Nov. 1, 2018; WO2020086730, entitled “Alk2 antibodies and methods of use thereof”, published Apr. 30, 2020; WO2020086963, entitled “Crystal forms of an alk2 inhibitor”, published Apr. 30, 2020; WO2020068729, entitled “Pyrazolo[4,3-d]pyrimidine compounds as alk2 and/or fgfr modulators”, published Apr. 2, 2020; US2020095250, entitled “Pyrazolopyrimidine compounds and uses thereof”, published Mar. 26, 2020; US2020199131, entitled “Imidazopyridazine and imidazopyridine compounds and uses thereof”, published Jun. 25, 2020, the contents of which are incorporated herein by reference. Still other suitable ALK2 inhibitors are disclosed in Hudson, L. et al., Novel Quinazolinone Inhibitors of ALK2 Flip between Alternate Binding Modes: Structure—Activity Relationship, Structural Characterization, Kinase Profiling, and Cellular Proof of Concept. Med. Chem. 2018, 61, 16, 7261-7272 and Carvalho D, et al., ALK2 inhibitors display beneficial effects in preclinical models of ACVR1 mutant diffuse intrinsic pontine glioma. Communications Biologyvolume 2, Article number: 156 (2019), the relevant contents of each of which are incorporated herein by reference. In some embodiments, a suitable ALK-2 inhibitor for use in the methods provided herein is KER-047. In some embodiments, a suitable ALK-2 inhibitor for use in the methods provided herein is BLU-782. In some embodiments, a suitable ALK-2 inhibitor for use in the methods provided herein is INCB000928. In some embodiments, the ALK2 inhibitor is LDN-212854, LDN-193189, or LDN-214117.

In some embodiments, the BMP antagonist is a BMP ligand trap. In some embodiments, a BMP ligand trap is a soluble BMP receptor. In some embodiments, the soluble BMP receptor is fused to an Fc portion of an immunoglobulin (e.g., a ActRIIa-Fc ligand trap or dalantercept, an activin receptor-like kinase-1 ligand trap, a ActRIIb-Fc ligand trap). Inhibition of BMP signaling by inhibiting BMP receptors is described in, e.g., Gomez-Puerto M C, et al., Bone morphogenetic protein receptor signal transduction in human disease. J Pathol. 2019 January; 247(1): 9-20. In some embodiments, the BMP ligand trap is a BMP ligand trap as disclosed in U.S. Pat. No. 7,709,605B2, entitled “ActRII receptor polypeptides, methods and compositions”, issued May 4, 2010, U.S. Pat. No. 9,526,759, entitled “Activin-actriia antagonists and uses for treating or preventing breast cancer”, issued Dec. 27, 2016, U.S. Pat. No. 8,058,229, entitled “A method of increasing red blood cell levels or treating anemia in a patient”, issued Nov. 15, 2011, US2013243743, entitled “Methods and compositions for treating ineffective erythropoiesis”, published Sep. 19, 2013, U.S. Ser. No. 10/307,455, entitled “Activin Type 2 Receptor Antibodies”, issued Jun. 4, 2019, U.S. Pat. No. 7,988,973, entitled “Activin-ActRII antagonists and uses for increasing red blood cell levels”, issued Aug. 2, 2011, U.S. Pat. No. 7,612,041, entitled “An isolated activing-binding ActRIIA polypeptide comprising the SEQ ID NO: 7 and uses for promoting bone growth”, issued Nov. 3, 2009, US2011070233 A1, entitled “Actriib antagonists and dosing and uses thereof”, published Mar. 24, 2011, U.S. Pat. No. 7,960,343, entitled “Activin-actriia antagonists and uses for decreasing or inhibiting FSH secretion”, issued Jun. 14, 2011, US2019282663, entitled “Activin receptor type iia variants and methods of use thereof”, published Sep. 19, 2019, WO2019094751, entitled “Activin receptor type iia variants and methods of use thereof”, published May 16, 2019, U.S. Pat. No. 7,842,663, entitled “Variants derived from ACTRIIB and uses therefor”, issued Nov. 30, 2010, US2010008918, published Jan. 14, 2010, U.S. Pat. No. 8,058,229, entitled “A method of increasing red blood cell levels or treating anemia in a patient”, issued Nov. 15, 2011, U.S. Pat. No. 8,293,881, entitled “An isolated nucleic acid encoding a truncated actriib fusion protein”, issued Oct. 23, 2012, U.S. Pat. No. 8,765,385, entitled “Method of detection of neutralizing anti-actriib antibodies”, issued Jul. 1, 2014, US2015361163, entitled “Methods for increasing red blood cell levels and treating sickle-cell disease”, published Dec. 17, 2015, US2017274077, entitled “Methods for increasing red blood cell levels and treating ineffective erythropoiesis”, published Sep. 28, 2017, WO2018067740, entitled “Compositions and method for treating kidney disease”, published Apr. 12, 2018, US2020055919, entitled “Variant actriib proteins and uses thereof”, published Feb. 20, 2020, WO2020092523, entitled “Treatment of anemia due to very low, low, or intermediate risk myelodysplastic syndromes in subjects with ring sideroblasts using activing-actrii ligand traps”, published May 7, 2020, U.S. Ser. No. 10/189,882, entitled “Methods for treating myelodysplastic syndromes and sideroblastic anemias”, issued Jan. 29, 2019, WO2019/140283, entitled “Activin receptor type iib variants and methods of use thereof”, published Jul. 18, 2019, U.S. Pat. No. 8,710,016, entitled “Actriib proteins and variants and uses therefore relating to utrophin induction for muscular dystrophy therapy”, issued Apr. 29, 2014, US2020/101134, entitled “Methods for treating myeloproliferative neoplasm-associated myelofibrosis and anemia”, published Apr. 2, 2020, US2018/148491, entitled “Novel Hybrid ActRIIB Ligand Trap Proteins For Treating Muscle Wasting Diseases”, published May 31, 2018, U.S. Pat. No. 9,884,900, entitled “Methods for treating janus kinase-associated disorders by administering soluble transforming growth factor beta type II receptor”, issued Feb. 6, 2018, the contents of each of which are incorporated herein by reference.

In some embodiments, the BMP antagonist is a dead BMP receptor. In some embodiments, the dead BMP receptor is a dominant negative BMP receptor. In some embodiments, overexpression of dead-BMP receptor interfered with BMP induced Smad activity. Any of the dominant negative BMP receptor can be used herein, e.g., Pouliot et al., Overexpression of a Dominant Negative Type II Bone Morphogenetic Protein Receptor Inhibits the Growth of Human Breast Cancer Cells, Cancer Res. 2003 Jan. 15; 63(2):277-81; Kawakami Y et al., BMP signaling during bone pattern determination in the developing limb. Development. 1996 November; 122(11):3557-66; Chen et al., Differential roles for bone morphogenetic protein (BMP) receptor type IB and IA in differentiation and specification of mesenchymal precursor cells to osteoblast and adipocyte lineages, J Cell Biol. 1998 Jul. 13; 142(1):295-305.

In some embodiments, a HJV-induced BMP signaling antagonist of the present disclosure is a hemojuvelin antagonist. In some embodiments, the hemojuvelin antagonist binds to one or more proteins of the repulsive guidance molecule (RGM) family, including RGMa, RGMb, and RGMc (HJV). In some embodiments, the hemojuvelin antagonist selectively binds hemojuvelin (RGMc) over RGMa and RGMb. In some embodiments, the hemojuvelin antagonist is an antisense oligonucleotide that reduces expression of hemojuvelin (see, e.g., U.S. Pat. No. 7,534,764, entitled “Competitive regulation of hepcidin mRNA by soluble and cell-associated hemojuvelin,” issued May 19, 2009; US2014127325, entitled “Competitive regulation of hepcidin mRNA by soluble and cell-associated hemojuvelin”, issued May 19, 2009; and WO2016180784, entitled “Improved treatments using oligonucleotides”, published Nov. 17, 2016, which are incorporated herein by reference). In some embodiments, the hemojuvelin antagonist is a small molecule compound that inhibits hemojuvelin, e.g., by competitive binding and/or chemical modification of hemojuvelin.

In some embodiments, the BMP antagonist is a HJV antagonist. In some embodiments, the HJV antagonist is a soluble HJV. In some embodiments, the soluble HJV is a soluble HJV-Fc fusion protein. In some embodiments, the soluble HJV is a soluble HJV as disclosed in U.S. Pat. No. 8,318,167B2, entitled “Methods and compositions for regulating iron homeostasis by modulation of BMP-6”. issued Nov. 27, 2012; U.S. Pat. No. 9,708,379B2, entitled “COMPOSITIONS FOR REGULATING IRON HOMEOSTASIS AND METHODS OF USING SAME,” issued Jul. 18, 2017, U.S. Ser. No. 10/273,273B2, entitled “COMPOSITIONS AND REGULATING IRON HOMEOSTASIS AND METHODS OF USING SAME,” issued Apr. 30, 2019, U.S. Pat. No. 7,968,091B2, entitled “METHODS AND COMPOSITIONS TO REGULATE IRON METABOLISM,” issued Jun. 28, 2011, U.S. Pat. No. 8,637,023B2, entitled “HEMOJUVELIN FUSION PROTEINS,” issued Jan. 28, 2014, U.S. Pat. No. 8,865,168B2, entitled “METHODS AND COMPOSITIONS TO REGULATE HEPCIDIN EXPRESSION,” issued Oct. 21, 2014, U.S. Pat. No. 9,556,251B2, entitled “METHODS AND COMPOSITIONS TO REGULATE HEPCIDIN EXPRESSION,” issued Jan. 31, 2017; U.S. Pat. No. 8,895,002B2, entitled “Hemojuvelin fusion proteins and uses thereof”, issued Nov. 25, 2014; U.S. Pat. No. 7,511,018B2, entitled “Juvenile hemochromatosis gene (HFE2A) cleavage products and uses thereof”, issued Mar. 31, 2009, the contents of each of which are incorporated herein by reference. In some embodiments, the sHJV-Fc fusion protein is Ferruxmax. In some embodiments, the sHJV-Fc fusion protein is FMX-8.

In some embodiments, a HJV-induced BMP signaling antagonist of the present disclosure is a hemojuvelin antagonist. In some embodiments, the hemojuvelin antagonist binds to one or more proteins of the repulsive guidance molecule (RGM) family, including RGMa, RGMb, and RGMc (HJV). In some embodiments, the hemojuvelin antagonist selectively binds hemojuvelin (RGMc) over RGMa and RGMb. In some embodiments, the hemojuvelin antagonist is an antisense oligonucleotide that reduces expression of hemojuvelin (see, e.g., U.S. Pat. No. 7,534,764, entitled “Competitive regulation of hepcidin mRNA by soluble and cell-associated hemojuvelin,” issued May 19, 2009; US2014127325, entitled “Competitive regulation of hepcidin mRNA by soluble and cell-associated hemojuvelin”, issued May 19, 2009; and WO2016180784, entitled “Improved treatments using oligonucleotides”, published Nov. 17, 2016, which are incorporated herein by reference). In some embodiments, the hemojuvelin antagonist is a small molecule compound that inhibits hemojuvelin, e.g., by competitive binding and/or chemical modification of hemojuvelin.

In some embodiments, the HJV-induced BMP signaling antagonist is a HJV antagonist. In some embodiments, the HJV antagonist is a soluble HJV. In some embodiments, the soluble HJV is a soluble HJV-Fc fusion protein. In some embodiments, the soluble HJV is a soluble HJV as disclosed in U.S. Pat. No. 8,318,167B2, entitled “Methods and compositions for regulating iron homeostasis by modulation of BMP-6”. issued Nov. 27, 2012; U.S. Pat. No. 9,708,379B2, entitled “COMPOSITIONS FOR REGULATING IRON HOMEOSTASIS AND METHODS OF USING SAME,” issued Jul. 18, 2017, U.S. Ser. No. 10/273,273B2, entitled “COMPOSITIONS AND REGULATING IRON HOMEOSTASIS AND METHODS OF USING SAME,” issued Apr. 30, 2019, U.S. Pat. No. 7,968,091B2, entitled “METHODS AND COMPOSITIONS TO REGULATE IRON METABOLISM,” issued Jun. 28, 2011, U.S. Pat. No. 8,637,023B2, entitled “HEMOJUVELIN FUSION PROTEINS,” issued Jan. 28, 2014, U.S. Pat. No. 8,865,168B2, entitled “METHODS AND COMPOSITIONS TO REGULATE HEPCIDIN EXPRESSION,” issued Oct. 21, 2014, U.S. Pat. No. 9,556,251B2, entitled “METHODS AND COMPOSITIONS TO REGULATE HEPCIDIN EXPRESSION,” issued Jan. 31, 2017; U.S. Pat. No. 8,895,002B2, entitled “Hemojuvelin fusion proteins and uses thereof”, issued Nov. 25, 2014; U.S. Pat. No. 7,511,018B2, entitled “Juvenile hemochromatosis gene (HFE2A) cleavage products and uses thereof”, issued Mar. 31, 2009, the contents of each of which are incorporated herein by reference. In some embodiments, the sHJV-Fc fusion protein is Ferruxmax. In some embodiments, the sHJV-Fc fusion protein is FMX-8.

In some embodiments, the hemojuvelin antagonist is an antibody specific for hemojuvelin and/or one or more proteins of the RGM protein family (e.g., RGMa, RGMb). In some embodiments, antibodies specific for hemojuvelin and/or one or more RGM proteins is an anti-HJV antibody and/or one or more RGM proteins as disclosed in U.S. Ser. No. 10/118,958, entitled “Composition and method for the diagnosis and treatment of iron-related disorders”, issued Nov. 6, 2018; U.S. Pat. No. 9,636,398, entitled “Composition and method for the diagnosis and treatment of iron-related disorders”, issued May 2, 2017; and U.S. Pat. No. 8,507,435, entitled “Juvenile hemochromatosis gene (HFE2A) cleavage products and uses thereof”, issued Aug. 13, 2013; U.S. Ser. No. 10/118,958, entitled “Composition and method for the diagnosis and treatment of iron-related disorders”, issued Nov. 6, 2018; US2010/0322941, entitled “Bone morphogenetic protein (BMP)-binding domains of proteins of the repulsive guidance molecule (RGM) protein family and functional fragments thereof, and use of same”, published Dec. 23, 2010; U.S. Pat. No. 9,040,052, entitled “Precision Medicine By Targeting Rare Human PCSK9 Variants for Cholesterol Treatment”, issued May 26, 2015; and US2017/0029499, entitled “Methods for treating hepcidin-mediated disorders”, published Feb. 2, 2017; and International Publication Nos. WO2007039256, entitled “Binding domains of proteins of the repulsive guidance molecule (rgm) protein family and functional fragments thereof, and their use,” published Apr. 12, 2007; WO2015171691, entitled “Compositions and methods for growth factor modulation”, published Nov. 12, 2015; WO2018/009624, entitled “Tgf-beta superfamily heteromultimers and uses thereof”, published Jan. 11, 2018, and WO2020/086736, entitled “Rgmc-selective inhibitors and use thereof”, published Apr. 30, 2020, the contents of each of which are incorporated herein by reference.

In some embodiments, the anti-HJV antibody is an anti-HJV antibody listed in Table 1. Table 1 contains example amino acid sequences of CDRs of anti-HJV antibodies. In some embodiments, a hepcidin antagonist of the present application is an anti-HJV antibody that comprises a CDR comprising an amino acid sequence selected from Table 1.

In some embodiments, the anti-HJV antibodies of the present disclosure comprises one or more of the heavy chain CDRs (e.g., CDR-H1, CDR-H2, or CDR-H3) amino acid sequences from any one of the anti-HJV antibodies selected from Table 1. In some embodiments, the anti-HJV antibodies of the present disclosure comprise the CDR-H1, CDR-H2, and CDR-H3 as provided for any one of the antibodies elected from Table 1. In some embodiments, the anti-HJV antibodies of the present disclosure comprises one or more of the light chain CDRs (e.g., CDR-L1, CDR-L2, or CDR-L3) amino acid sequences from any one of the anti-HJV antibodies selected from Table 1. In some embodiments, the anti-HJV antibodies of the present disclosure comprise the CDR-L1, CDR-L2, and CDR-L3 as provided for any one of the anti-HJV antibodies selected from Table 1.

In some embodiments, the anti-HJV antibodies of the present disclosure comprises the CDR-H1, CDR-H2, CDR-H3, CDR-L1, CDR-L2, and CDR-L3 as provided for any one of the anti-HJV antibodies selected from Table 1. In some embodiments, antibody heavy and light chain CDR3 domains may play a particularly important role in the binding specificity/affinity of an antibody for an antigen. Accordingly, the anti-HJV antibodies of the disclosure may include at least the heavy and/or light chain CDR3s of any one of the anti-HJV antibodies selected from Table 1.

Also within the scope of the present disclosure are functional variants of any of the exemplary anti-HJV antibodies as disclosed herein. A functional variant may contain one or more amino acid residue variations in the V_{H} and/or V_{L} as relative to the reference antibody, while retaining substantially similar binding and biological activities (e.g., substantially similar binding affinity, binding specificity, inhibitory activity, anti-inflammatory activity, or a combination thereof) as the reference antibody. In some embodiments, a functional variant of the anti-HJV antibody as described herein contains one or more amino acid variations in the heavy chain CDRs and/or one or more amino acid variation in the light chain CDRs as relative to the reference antibody, while retaining substantially similar binding and biological activities (e.g., substantially similar binding affinity, binding specificity, inhibitory activity, anti-inflammatory activity, or a combination thereof) as the reference antibody. In some embodiments, a functional variant of the anti-HJV antibody as described herein contains one or more amino acid variations in the heavy chain framework region and/or one or more amino acid variation in the light chain framework region as relative to the reference antibody, while retaining substantially similar binding and biological activities (e.g., substantially similar binding affinity, binding specificity, inhibitory activity, anti-inflammatory activity, or a combination thereof) as the reference antibody. Substantially, as used herein in the context of function (e.g., binding affinity and/or biological function), refers to an antibody variant (e.g., anti-HJV antibody variant) have at least 80%, at least 85%, at least 90%, at least 91%, at least 91%, at least 91%, at least 91%, at least 91%, at least 91%, at least 91%, at least 91%, at least 91%, or 100% function (e.g., binding affinity and/or biological function) as compared to the reference antibody (e.g., any of the anti-HJV antibody as described in table 1 and table 2).

In some embodiments, any of the anti-HJV antibodies of the disclosure have one or more CDRs (e.g., Heavy chain CDR or light chain CDR) sequences substantially similar to any of the CDR-H1, CDR-H2, CDR-H3, CDR-L1, CDR-L2, and/or CDR-L3 sequences from one of the anti-HJV antibodies selected from Table 1. In some embodiments, the position of one or more CDRs along the VH (e.g., CDR-H1, CDR-H2, or CDR-H3) and/or VL (e.g., CDR-L1, CDR-L2, or CDR-L3) region of an antibody described herein can vary by one, two, three, four, five, or six amino acid positions so long as immunospecific binding to hemojuvelin (e.g., human hemojuvelin) is maintained (e.g., substantially maintained, for example, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as immunospecific binding to hemojuvelin (e.g., human hemojuvelin) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1, CDR-H2, or CDR-H3) and/or VL (e.g., CDR-L1, CDR-L2, or CDR-L3) region of an antibody described herein can vary (e.g., be shorter or longer) by one, two, three, four, five, or more amino acids, so long as immunospecific binding to hemojuvelin (e.g., human hemojuvelin) is maintained (e.g., substantially maintained, for example, at least 50%, at least 60%, at least 70%, at least 80%, at least 90%, at least 95% of the binding of the original antibody from which it is derived).

Accordingly, in some embodiments, a HC CDR1, HC CDR2, HC CDR3, LC CDR1, LC CDR2, and/or LC CDR3 described herein may be one, two, three, four, five or more amino acids shorter than one or more of the CDRs described herein (e.g., CDRs from any of the anti-HJV antibodies selected from Table 1) so long as immunospecific binding to hemojuvelin (e.g., human hemojuvelin) is maintained (e.g., substantially maintained, for example, at least 80%, at least 90%, at least 95% relative to the binding of the original antibody from which it is derived). In some embodiments, a HC CDR1, HC CDR2, HC CDR3, LC CDR1, LC CDR2, and/or LC CDR3 described herein may be one, two, three, four, five or more amino acids longer than one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRs from any of the anti-HJV antibodies selected from Table 1) so long as immunospecific binding to hemojuvelin (e.g., human hemojuvelin) is maintained (e.g., substantially maintained, for example, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRs from any of the anti-HJV antibodies selected from Table 1) so long as immunospecific binding to hemojuvelin (e.g., human hemojuvelin) is maintained (e.g., substantially maintained, for example, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRs from any of the anti-HJV antibodies selected from Table 1) so long as immunospecific binding to hemojuvelin (e.g., human hemojuvelin) is maintained (e.g., substantially maintained, for example, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRs from any of the anti-HJV antibodies selected from Table 1) so long as immunospecific binding to hemojuvelin (e.g., human hemojuvelin) is maintained (e.g., substantially maintained, for example, at least 80%, at least 90%, at least 95% relative to the binding of the original antibody from which it is derived). Any suitable known method can be used to ascertain whether immunospecific binding to hemojuvelin (e.g., human hemojuvelin) is maintained, for example, using binding assays and conditions described in the art.

In some examples, any of the anti-HJV antibodies of the disclosure have one or more CDR (e.g., HC CDR or LC CDR) sequences substantially similar to any one of the anti-HJV antibodies selected from Table 1. For example, the antibodies may include one or more CDR sequence(s) from any of the anti-HJV antibodies selected from Table 1 containing up to 5, 4, 3, 2, or 1 amino acid residue variations as compared to the corresponding CDR region in any one of the CDRs provided herein (e.g., CDRs from any of the anti-HJV antibodies selected from Table 1) so long as immunospecific binding to hemojuvelin (e.g., human hemojuvelin) is maintained (e.g., substantially maintained, for example, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a hemojuvelin protein (e.g., a human hemojuvelin protein), for example, as determined based on a crystal structure. Some aspects of the disclosure provide anti-HJV antibodies that comprise one or more of the heavy chain variable (VH) and/or light chain variable (VL) domains provided herein. In some embodiments, any of the VH domains provided herein include one or more of the HC CDR sequences (e.g., HC CDR1, HC CDR2, and HC CDR3) provided herein, for example, any of the CDR-H sequences provided in any one of the anti-HJV selected from Table 1. In some embodiments, any of the VL domains provided herein include one or more of the CDR-L sequences (e.g., LC CDR1, LC CDR2, and LC CDR3) provided herein, for example, any of the LC CDR sequences provided in any one of the anti-HJV antibodies selected from Table 1.

In some embodiments, an anti-HJV antibody comprises a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3. In some embodiments, the anti-HJV antibody comprises a variable light chain region comprising a CDR1 comprising an amino acid sequence selected from any one of SEQ ID NOs: 4, 7, 10, 13, and 16, a CDR2 comprising an amino acid sequence selected from any one of SEQ ID NOs: 5, 8, 11, 14, and 17, and a CDR3 comprising an amino acid sequence selected from any one of SEQ ID NOs: 6, 9, 12, 15, and 18.

In some embodiments, an anti-HJV antibody comprises a variable heavy chain region comprising a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21. In some embodiments, the anti-HJV antibody comprises a variable light chain region comprising a CDR1 comprising the amino acid sequence of SEQ ID NO: 22, a CDR2 comprising the amino acid sequence of SEQ ID NO: 23, and a CDR3 comprising the amino acid sequence of SEQ ID NO: 24.

TABLE 1
Anti-Hemojuvelin Complementarity
determining region (CDR) Sequences

| | SEQ | |
|---|---|---|
| | ID | |
| Name | NO | SEQUENCE |
| CDR-H1 | 1 | NYGMN |
| CDR-H2 | 2 | MIYYDSSEKHYADSVKG |
| CDR-H3 | 3 | GTTPDY |
| CDR-L1 | 4 | RSSQSLESSDGDTFLE |
| CDR-L2 | 5 | DVSTRFS |
| CDR-L3 | 6 | FQVTHDPVT |
| CDR-L1 | 7 | RSSQSLEESDGYTFLH |
| CDR-L2 | 8 | EVSTRFS |
| CDR-L3 | 9 | FQATHDPLT |
| CDR-L1 | 10 | RSSQSLADSDGDTFLH |
| CDR-L2 | 11 | AVSHRFS |
| CDR-L3 | 12 | FQATHDPVT |
| CDR-L1 | 13 | RSSQSLEDSDGGTFLE |
| CDR-L2 | 14 | DVSSRFS |
| CDR-L3 | 15 | FQATHDPLS |
| CDR-L1 | 16 | RSSQSLEYSDGYTFLE |
| CDR-L2 | 17 | EVSNRFS |
| CDR-L3 | 18 | FQATHDPLT |
| CDR-H1 | 19 | GFNIRDFYIH |
| CDR-H2 | 20 | WIDPENGDIEYAPKFQG |
| CDR-H3 | 21 | NGYYLDY |
| CDR-L1 | 22 | KSGQSLLHSDGKTYLN |
| CDR-L2 | 23 | LVSKLDS |
| CDR-L3 | 24 | WQGTHSPWT |

In some embodiments, the anti-HJV antibodies of the disclosure include any antibody that includes a heavy chain variable domain and/or a light chain variable domain of any one of the anti-HJV antibodies selected from Table 2, and variants thereof. In some embodiments, anti-HJV antibodies of the disclosure include any antibody that includes the heavy chain variable and light chain variable pairs of any anti-HJV antibodies selected from Table 2.

Aspects of the disclosure provide anti-HJV antibodies having a heavy chain variable (VH) and/or a light chain variable (VL) domain amino acid sequence homologous to any of those described herein. In some embodiments, the anti-HJV antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any one of the anti-HJV antibodies selected from Table 2. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In some embodiments, any of the anti-HJV antibodies provided herein comprise a heavy chain variable sequence and a light chain variable sequence that comprises a framework sequence that is at least 75%, 80%, 85%, 90%, 95%, 98%, or 99% identical to the framework sequence of any anti-HJV antibodies selected from Table 2.

Table 2 contains example amino acid sequences for variable heavy chain and variable light chain anti-HJV antibodies. In some embodiments, a hepcidin antagonist of the present application is an anti-HJV antibody that comprises a variable heavy chain and/or a variable light chain comprising an amino acid sequence selected from Table 2.

In some embodiments, the anti-HJV antibody of the present disclosure comprises a CDR-H1, CDR-H2 and CDR-H3 of a heavy chain variable domain having the amino acid sequence of SEQ ID NO: 25. Alternatively or in addition, the anti-HJV antibody of the present disclosure comprises a CDR-L1, CDR-L2 and CDR-L3 of a light chain variable domain having the amino acid sequence of SEQ ID NO: 26.

In some embodiments, according to the Kabat definition system, the anti-HJV antibody of the present disclosure comprises a CDR-H1 having the amino acid sequence of SEQ ID NO: 1, a CDR-H2 having the amino acid sequence of SEQ ID NO: 2, a CDR-H3 having the amino acid sequence of SEQ ID NO: 3; and/or a CDR-L1 having the amino acid sequence of SEQ ID NO: 4, a CDR-L2 having the amino acid sequence of SEQ ID NO: 5, and a CDR-L3 having the amino acid sequence of SEQ ID NO: 6. In some embodiments, an anti-HJV antibody comprises a variable heavy chain region comprising the amino acid sequence of SEQ ID NO: 25, and/or a variable light chain region comprising the amino acid sequence of SEQ ID NO: 26.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 25. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 26. In some embodiments, the anti-HJV antibody of the present disclosure comprises a VH comprising an amino acid sequence that is at least 80% (e.g., 80%, 85%, 90%, 95%, 98%, or 99%) identical to the VH as set forth in SEQ ID NO: 25. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 26. In some embodiments, the function of an anti-HJV antibody having amino acid variations in the FR region of VH (e.g., based on Kabat definition) as compared to the VH as set forth in SEQ ID NO: 25 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VH (e.g., based on Kabat definition) as compared to the VH as set forth in SEQ ID NO: 25 is maintained (e.g., substantially maintained, for example, at least 80%, at least 90%, at least 95% of the binding of the original antibody from which it is derived). Alternatively or in addition, in some embodiments, the function of an anti-HJV antibody having amino acid variations in the FR region of VL (e.g., based on Kabat definition) as compared to the VL as set forth in SEQ ID NO: 26 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VL (e.g., based on Kabat definition) as compared to the VL as set forth in SEQ ID NO: 26 is maintained (e.g., substantially maintained, for example, at least 80%, at least 90%, at least 95% of the binding of the original antibody from which it is derived).

In some embodiments, the anti-HJV antibody of the present disclosure comprises a CDR-H1, CDR-H2 and CDR-H3 of a heavy chain variable domain having the amino acid sequence of SEQ ID NO: 27. Alternatively or in addition, the anti-HJV antibody of the present disclosure comprises a CDR-L1, CDR-L2 and CDR-L3 of a light chain variable domain having the amino acid sequence of SEQ ID NO: 28.

In some embodiments, according to the Kabat definition system, the anti-HJV antibody of the present disclosure comprises a CDR-H1 having the amino acid sequence of SEQ ID NO: 1, a CDR-H2 having the amino acid sequence of SEQ ID NO: 2, a CDR-H3 having the amino acid sequence of SEQ ID NO: 3; and/or a CDR-L1 having the amino acid sequence of SEQ ID NO: 7, a CDR-L2 having the amino acid sequence of SEQ ID NO: 8, and a CDR-L3 having the amino acid sequence of SEQ ID NO: 9.

In some embodiments, an anti-HJV antibody comprises a variable heavy chain region comprising the amino acid sequence of SEQ ID NO: 27, and/or a variable light chain region comprising the amino acid sequence of SEQ ID NO: 28.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 27. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 28. In some embodiments, the anti-HJV antibody of the present disclosure comprises a VH comprising an amino acid sequence that is at least 80% (e.g., 80%, 85%, 90%, 95%, 98%, or 99%) identical to the VH as set forth in SEQ ID NO: 27. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 28. In some embodiments, the function of an anti-HJV antibody having amino acid variations in the FR region of VH (e.g., based on Kabat definition) as compared to the VH as set forth in SEQ ID NO: 27 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VH (e.g., based on Kabat definition) as compared to the VH as set forth in SEQ ID NO: 27 is maintained (e.g., substantially maintained, for example, at least 80%, at least 90%, at least 95% of the binding of the original antibody from which it is derived). Alternatively or in addition, in some embodiments, the function of an anti-HJV antibody having amino acid variations in the FR region of VL (e.g., based on Kabat definition) as compared to the VL as set forth in SEQ ID NO: 28 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VL (e.g., based on Kabat definition) as compared to the VL as set forth in SEQ ID NO: 28 is maintained (e.g., substantially maintained, for example, at least 80%, at least 90%, at least 95% of the binding of the original antibody from which it is derived).

In some embodiments, the anti-HJV antibody of the present disclosure comprises a CDR-H1, CDR-H2 and CDR-H3 of a heavy chain variable domain having the amino acid sequence of SEQ ID NO: 29. Alternatively or in addition, the anti-HJV antibody of the present disclosure comprises a CDR-L1, CDR-L2 and CDR-L3 of a light chain variable domain having the amino acid sequence of SEQ ID NO: 30.

In some embodiments, according to the Kabat definition system, the anti-HJV antibody of the present disclosure comprises a CDR-H1 having the amino acid sequence of SEQ ID NO: 1, a CDR-H2 having the amino acid sequence of SEQ ID NO: 2, a CDR-H3 having the amino acid sequence of SEQ ID NO: 3; and/or a CDR-L1 having the amino acid sequence of SEQ ID NO: 10, a CDR-L2 having the amino acid sequence of SEQ ID NO: 11, and a CDR-L3 having the amino acid sequence of SEQ ID NO: 12.

In some embodiments, an anti-HJV antibody comprises a variable heavy chain region comprising the amino acid sequence of SEQ ID NO: 29, and/or a variable light chain region comprising the amino acid sequence of SEQ ID NO: 30.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 29. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 30. In some embodiments, the anti-HJV antibody of the present disclosure comprises a VH comprising an amino acid sequence that is at least 80% (e.g., 80%, 85%, 90%, 95%, 98%, or 99%) identical to the VH as set forth in SEQ ID NO: 29. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 30. In some embodiments, the function of an anti-HJV antibody having amino acid variations in the FR region of VH (e.g., based on Kabat definition) as compared to the VH as set forth in SEQ ID NO: 29 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VH (e.g., based on Kabat definition) as compared to the VH as set forth in SEQ ID NO: 29 is maintained (e.g., substantially maintained, for example, at least 80%, at least 90%, at least 95% of the binding of the original antibody from which it is derived). Alternatively or in addition, in some embodiments, the function of an anti-HJV antibody having amino acid variations in the FR region of VL (e.g., based on Kabat definition) as compared to the VL as set forth in SEQ ID NO: 30 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VL (e.g., based on Kabat definition) as compared to the VL as set forth in SEQ ID NO: 30 is maintained (e.g., substantially maintained, for example, at least 80%, at least 90%, at least 95% of the binding of the original antibody from which it is derived).

In some embodiments, the anti-HJV antibody of the present disclosure comprises a CDR-H1, CDR-H2 and CDR-H3 of a heavy chain variable domain having the amino acid sequence of SEQ ID NO: 31. Alternatively or in addition, the anti-HJV antibody of the present disclosure comprises a CDR-L1, CDR-L2 and CDR-L3 of a light chain variable domain having the amino acid sequence of SEQ ID NO: 32.

In some embodiments, according to the Kabat definition system, the anti-HJV antibody of the present disclosure comprises a CDR-H1 having the amino acid sequence of SEQ ID NO: 1, a CDR-H2 having the amino acid sequence of SEQ ID NO: 2, a CDR-H3 having the amino acid sequence of SEQ ID NO: 3; and/or a CDR-L1 having the amino acid sequence of SEQ ID NO: 13, a CDR-L2 having the amino acid sequence of SEQ ID NO: 14, and a CDR-L3 having the amino acid sequence of SEQ ID NO: 15.

In some embodiments, an anti-HJV antibody comprises a variable heavy chain region comprising the amino acid sequence of SEQ ID NO: 31, and/or a variable light chain region comprising the amino acid sequence of SEQ ID NO: 32.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 31. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 32. In some embodiments, the anti-HJV antibody of the present disclosure comprises a VH comprising an amino acid sequence that is at least 80% (e.g., 80%, 85%, 90%, 95%, 98%, or 99%) identical to the VH as set forth in SEQ ID NO: 31. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 32. In some embodiments, the function of an anti-HJV antibody having amino acid variations in the FR region of VH (e.g., based on Kabat definition) as compared to the VH as set forth in SEQ ID NO: 31 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VH (e.g., based on Kabat definition) as compared to the VH as set forth in SEQ ID NO: 31 is maintained (e.g., substantially maintained, for example, at least 80%, at least 90%, at least 95% of the binding of the original antibody from which it is derived). Alternatively or in addition, in some embodiments, the function of an anti-HJV antibody having amino acid variations in the FR region of VL (e.g., based on Kabat definition) as compared to the VL as set forth in SEQ ID NO: 32 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VL (e.g., based on Kabat definition) as compared to the VL as set forth in SEQ ID NO: 32 is maintained (e.g., substantially maintained, for example, at least 80%, at least 90%, at least 95% of the binding of the original antibody from which it is derived).

In some embodiments, the anti-HJV antibody of the present disclosure comprises a CDR-H1, CDR-H2 and CDR-H3 of a heavy chain variable domain having the amino acid sequence of SEQ ID NO: 33. Alternatively or in addition, the anti-HJV antibody of the present disclosure comprises a CDR-L1, CDR-L2 and CDR-L3 of a light chain variable domain having the amino acid sequence of SEQ ID NO: 34.

In some embodiments, according to the Kabat definition system, the anti-HJV antibody of the present disclosure comprises a CDR-H1 having the amino acid sequence of SEQ ID NO: 1, a CDR-H2 having the amino acid sequence of SEQ ID NO: 2, a CDR-H3 having the amino acid sequence of SEQ ID NO: 3; and/or a CDR-L1 having the amino acid sequence of SEQ ID NO: 16, a CDR-L2 having the amino acid sequence of SEQ ID NO: 17, and a CDR-L3 having the amino acid sequence of SEQ ID NO: 18.

In some embodiments, an anti-HJV antibody comprises a variable heavy chain region comprising the amino acid sequence of SEQ ID NO: 33, and/or a variable light chain region comprising the amino acid sequence of SEQ ID NO: 34.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 33. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 34. In some embodiments, the anti-HJV antibody of the present disclosure comprises a VH comprising an amino acid sequence that is at least 80% (e.g., 80%, 85%, 90%, 95%, 98%, or 99%) identical to the VH as set forth in SEQ ID NO: 33. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 34. In some embodiments, the function of an anti-HJV antibody having amino acid variations in the FR region of VH (e.g., based on Kabat definition) as compared to the VH as set forth in SEQ ID NO: 33 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VH (e.g., based on Kabat definition) as compared to the VH as set forth in SEQ ID NO: 33 is maintained (e.g., substantially maintained, for example, at least 80%, at least 90%, at least 95% of the binding of the original antibody from which it is derived). Alternatively or in addition, in some embodiments, the function of an anti-HJV antibody having amino acid variations in the FR region of VL (e.g., based on Kabat definition) as compared to the VL as set forth in SEQ ID NO: 34 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VL (e.g., based on Kabat definition) as compared to the VL as set forth in SEQ ID NO: 34 is maintained (e.g., substantially maintained, for example, at least 80%, at least 90%, at least 95% of the binding of the original antibody from which it is derived).

In some embodiments, the anti-HJV antibody of the present disclosure comprises a CDR-H1, CDR-H2 and CDR-H3 of a heavy chain variable domain having the amino acid sequence of SEQ ID NO: 35. Alternatively or in addition, the anti-HJV antibody of the present disclosure comprises a CDR-L1, CDR-L2 and CDR-L3 of a light chain variable domain having the amino acid sequence of SEQ ID NO: 36.

In some embodiments, according to the Kabat definition system, the anti-HJV antibody of the present disclosure comprises a CDR-H1 having the amino acid sequence of SEQ ID NO: 19, a CDR-H2 having the amino acid sequence of SEQ ID NO: 20, a CDR-H3 having the amino acid sequence of SEQ ID NO: 21; and/or a CDR-L1 having the amino acid sequence of SEQ ID NO: 22, a CDR-L2 having the amino acid sequence of SEQ ID NO: 23, and a CDR-L3 having the amino acid sequence of SEQ ID NO: 24.

In some embodiments, an anti-HJV antibody comprises a variable heavy chain region comprising the amino acid sequence of SEQ ID NO: 35, and/or a variable light chain region comprising the amino acid sequence of SEQ ID NO: 36.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 35. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 36. In some embodiments, the anti-HJV antibody of the present disclosure comprises a VH comprising an amino acid sequence that is at least 80% (e.g., 80%, 85%, 90%, 95%, 98%, or 99%) identical to the VH as set forth in SEQ ID NO: 35. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 36. In some embodiments, the function of an anti-HJV antibody having amino acid variations in the FR region of VH (e.g., based on Kabat definition) as compared to the VH as set forth in SEQ ID NO: 35 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VH (e.g., based on Kabat definition) as compared to the VH as set forth in SEQ ID NO: 35 is maintained (e.g., substantially maintained, for example, at least 80%, at least 90%, at least 95% of the binding of the original antibody from which it is derived). Alternatively or in addition, in some embodiments, the function of an anti-HJV antibody having amino acid variations in the FR region of VL (e.g., based on Kabat definition) as compared to the VL as set forth in SEQ ID NO: 36 is maintained (e.g., substantially maintained, for example, at least 80%, at least 90%, at least 95% of the binding of the original antibody from which it is derived). In some embodiments, the function of an anti-HJV antibody having no more than 5 (e.g., no more than 5, 4, 3, 2 or 1), no more than 3 (e.g., no more than 3, 2, or 1) amino acid variations in the FR region of VL (e.g., based on Kabat definition) as compared to the VL as set forth in SEQ ID NO: 36 is maintained (e.g., substantially maintained, for example, at least 80%, at least 90%, at least 95% of the binding of the original antibody from which it is derived).

TABLE 2
Anti-Hemojuvelin Variable Heavy Chain (V_{H}) and
Variable Light Chain (V_{L}) Sequences

| Name | SEQ ID NO | SEQUENCE |
|---|---|---|
| V_{H} | 25 | EVQLVESGGGLVQPGGSLRLSCAASGFTFSNYGMNWIRQAPGKGLEWIGMI |
| | | YYDSSEKHYADSVKGRFTISRDNSKNTLYLQMNSLRAEDTAVYYCAKGTTP |
| | | DYWGQGTMVTVSS |
| V_{L} | 26 | DVVLTQSPLSLPVTLGQPASISCRSSQSLESSDGDTFLEWFQQRPGQSPRL |
| | | LIYDVSTRFSGVPDRFSGSGSGTDFTLKISRVEAEDVGVYYCFQVTHDPVT |
| | | FGQGTKLEIK |
| V_{H} | 27 | EVQLVESGGGLVQPGGSLRLSCAASGFTFSNYGMNWIRQAPGKGLEWIGMI |
| | | YYDSSEKHYADSVKGRFTISRDNSKNTLYLQMNSLRAEDTAVYYCAKGTTP |
| | | DYWGQGTMVTVSS |
| V_{L} | 28 | DVVLTQSPLSLPVTLGQPASISCRSSQSLEESDGYTFLHWFQQRPGQSPRL |
| | | LIYEVSTRFSGVPDRFSGSGSGTDFTLKISRVEAEDVGVYYCFQATHDPLT |
| | | FGQGTKLEIK |
| V_{H} | 29 | EVQLVESGGGVVQPGRSLRLSCAASGFTFSNYGMNWVRQAPGKGLEWVAMI |
| | | YYDSSEKHYADSVKGRFTISRDNSKNTLYLQMNSLRAEDTAVYYCARGTTP |
| | | DYWGQGTMVTVSS |
| V_{L} | 30 | DVVLTQSPLSLPVTLGQPASISCRSSQSLADSDGDTFLHWFQQRPGQSPRL |
| | | LIYAVSHRFSGVPDRFSGSGSGTDFTLKISRVEAEDVGVYYCFQATHDPVT |
| | | FGQGTKLEIK |
| V_{H} | 31 | EVQLVESGGGLVQPGGSLRLSCAASGFTFSNYGMNWVRQAPGKGLEWVSMI |
| | | YYDSSEKHYADSVKGRFTISRDNSKNTLYLQMNSLRAEDTAVYYCAKGTTP |
| | | DYWGQGTMVTVSS |
| V_{L} | 32 | DVVLTQSPLSLPVTLGQPASISCRSSQSLEDSDGGTFLEWFQQRPGQSPRL |
| | | LIYDVSSRFSGVPDRFSGSGSGTDFTLKISRVEAEDVGVYYCFQATHDPLS |
| | | FGQGTKLEIK |
| V_{H} | 33 | EVQLVESGGGLVQPGGSLRLSCAASGFTFSNYGMNWIRQAPGKGLEWIGMI |
| | | YYDSSEKHYADSVKGRFTISRDNSKNTLYLQMNSLRAEDTAVYYCAKGTTP |
| | | DYWGQGTMVTVSS |
| V_{L} | 34 | DVVLTQSPLSLPVTLGQPASISCRSSQSLEYSDGYTFLEWFQQRPGQSPRL |
| | | LIYEVSNRFSGVPDRFSGSGSGTDFTLKISRVEAEDVGVYYCFQATHDPLT |
| | | FGQGTKLEIKR |
| V_{H} | 35 | EVQLQQSGAELVRSGASVKLSCTASGFNIRDFYIHWVKQRPEQGLEWLGWI |
| | | DPENGDIEYAPKFQGKATMTADTSSNTAYLQLNSLTSEDTALYYCNGNGYY |
| | | LDYWGQGTTLTVSS |
| V_{L} | 36 | DVVMTQTPLTLSVTIGQPASISCKSGQSLLHSDGKTYLNWLLQRPGQSPKR |
| | | LIYLVSKLDSGVPDRFTGSGSGTDFTLKISRVEAEDLGVYYCWQGTHSPWT |
| | | FGGGTKLEIKR |

In some embodiments, the anti-HJV antibodies described herein is capable of inhibiting hepcidin expression by blocking the BMP signaling pathway (e.g., BMP6 signaling pathway). In some embodiments, the anti-HJV antibodies described herein is capable of inhibiting hepcidin expression by blocking the JAK-STAT pathway (e.g., IL-6 signaling pathway). In some embodiments, the anti-HJV antibodies described herein is capable of inhibiting hepcidin expression by blocking the BMP signaling pathway (e.g., BMP6 signaling pathway) and the JAK-STAT pathway (e.g., IL-6 signaling pathway).

In some embodiments, the HJV antagonist is an inhibitory nucleic acid targeting HJV (e.g., dsRNA, siRNA, miRNA, shRNA, AmiRNA, antisense oligonucleotides (ASO) or aptamer targeting HJV). In some embodiments, an inhibitory nucleic acid targeting HJV can be used herein in treating ACD.

In some embodiments, the anti-HJV antagonist is recombinant matriptase-2 (TMPRSS6). Matriptase-2 is a transmembrane serine protease capable of cleaving HJV, and overexpression of matriptase-2 protein in cells suppresses the activation of hepcidin expression (Du X, She E, Gelbart T, et al. The serine protease TMPRSS6 is required to sense iron deficiency, Science, 2008, vol. 320 5879 (pg. 1088-1092).

In some embodiments, upon ligand binding, constitutively active type II receptors phosphorylate type I receptors, and type I receptors then phosphorylate intracellular receptor-activated Smads (R-Smads), namely Smad1, Smad5 and/or Smad8. In such embodiments, activated R-Smads complex with the common partner Smad4 and translocate to the nucleus to regulate gene transcription, e.g., induction of hepcidin expression. In some embodiments, the HJV-induced BMP signaling antagonist is an intracellular inhibitor for R-Smads (e.g., Smad 1, Smad 5, and Smad8) and the partner Smad4. In some embodiments, the intracellular inhibitors for the R-Smads and Smad4 are intracellular antibodies.

In some embodiments, the intracellular inhibitors for Smads are inhibitory nucleic acid targeting R-Smads (e.g., Smad1, Smad5, or Smad8) and Smad4. In some embodiments, the inhibitory nucleic acid targeting R-Smads (e.g., Smad1, Smad5, or Smad8) and Smad4 is an inhibitory RNA. In some embodiments, the inhibitory nucleic acid is a miRNA targeting R-Smads (e.g., Smad1, Smad5, or Smad8) and Smad4. In some embodiments, the inhibitory nucleic acid is a shRNA targeting R-Smads (e.g., Smad1, Smad5, or Smad8) and Smad4. In some embodiments, the inhibitory nucleic acid is a siRNA targeting R-Smads (e.g., Smad1, Smad5, or Smad8) and Smad4. In some embodiments, the inhibitory nucleic acid is an AmiRNA targeting R-Smads (e.g., Smad1, Smad5, or Smad8) and Smad4.

In some embodiments, the intracellular inhibitors for R-Smads and Smad4 is a recombinant inhibitory Smad (I-Smads) (e.g., Smad6 or Smad7). In some embodiments, Smad6 preferentially inhibits Smad signaling initiated by the bone morphogenetic protein (BMP) type I receptors ALK-3 and ALK-6, and Smad7 inhibits both transforming growth factor β(TGF-β)- and BMP-induced Smad signaling.

Efficient iron signaling via the BMP-SMAD signaling pathway involves auxiliary factors, such as the diferric transferrin (Tf) sensor transferrin receptor 2 (TfR) to stimulate hepcidin expression (FIG. 2). In some embodiments, a hepcidin antagonist of the present disclosure is a transferrin antagonist, which antagonizes hepcidin function by binding transferrin and/or transferrin receptor 2 to inhibit activation of the BMP-SMAD signaling pathway. In some embodiments, the transferrin antagonist is an antisense oligonucleotide targeting Tf and/or TfR, such as siTFR2 (see, e.g., U.S. Pat. No. 9,228,188, which is incorporated herein by reference).

ii. Other Hepcidin Antagonists

In some embodiments, the hepcidin antagonist is a hepcidin neutralizing agent. A hepcidin neutralizing agent, refers to an agent that directly neutralizes hepcidin. In some embodiments, a hepcidin neutralizing agent of the disclosure is an agent that binds HAMP or a transcription or translation product thereof. Examples of such hepcidin neutralizing agent include, without limitation, antisense oligonucleotides, small molecule inhibitor compounds, and antibodies, anticalins, or aptamers specific for a HAMP transcription or translation product (e.g., hepcidin).

In some aspects, a hepcidin neutralizing agent is a hepcidin inhibitor. In some embodiments, the hepcidin inhibitor is a molecule that specifically binds hepcidin (e.g., an antibody, an anticalin, or an aptamer). Examples of molecules that specifically bind hepcidin include, without limitation, PRS-080, LY2787106, NOX-H94 (Lexaptepid Pegol), 12B9m, LS-B4534, lipocalin mutein, and hNGAL mutein (see also U.S. Pat. Nos. 8,629,250; 9,315,577; 9,051,382; 9,657,098; 9,610,356; 8,530,619; and U.S. Patent Publication Nos. US 2015/0291675, US 2018/0057812, and US 2017/0247448, which are incorporated herein by reference).

In some embodiments, the hepcidin neutralizing agent is an anti-hepcidin antibody. In some embodiments, the anti-hepcidin antibody is an anti-hepcidin antibody as described in U.S. Ser. No. 10/323,088B2, entitled “Humanized anti-hepcidin antibodies and uses thereof,” issued Aug. 31, 2017; U.S. Pat. No. 8,609,817B2, entitled “Anti-hepcidin-25 selective antibodies and uses thereof”, issued Dec. 17, 2013, U.S. Pat. No. 8,304,258B2, entitled “Methods of producing monoclonal antibodies specific for human hepcidin”, issued Nov. 6, 2012, U.S. Pat. No. 8,629,250B2, entitled “Hepcidin, hepcidin antagonists and methods of use”, issued Jan. 14, 2014, U.S. Pat. No. 9,657,098B2, entitled “Anti-hepcidin antibodies and uses thereof”, issued May 23, 2017, U.S. Pat. No. 9,803,011B2, entitled “Anti-hepcidin antibodies and uses thereof”, issued Oct. 31, 2017, U.S. Ser. No. 10/239,941B2, entitled “Anti-hepcidin antibodies and uses thereof” issued Mar. 26, 2019, or U.S. Pat. No. 9,315,577B2, entitled “Anti-hepcidin antibodies and methods of use”, issued Apr. 19, 2016, which are incorporated herein by reference. In some embodiments, the anti-hepcidin antibody is LY2787106.

In some embodiments, the hepcidin neutralizing agent is an inhibitory nucleic acid targeting hepcidin. As a set of non-limiting examples, an inhibitory nucleic acid may be, but is not limited to, a small interfering RNA (siRNA), microRNA (miRNA), short hairpin RNA (shRNA), dicer substrate interfering RNA (dsiRNA), short siRNA, or single-stranded siRNA. In some embodiments, a double-stranded antisense oligonucleotide is an RNAi oligonucleotide. In some embodiments, the inhibitory nucleic acid targeting hepcidin include, without limitation, siHepcidin and XEN701 as disclosed in US20160186172, entitled “Compositions and methods for inhibiting hepcidin antimicrobial peptide (HAMP) or HAMP-related gene expression”, published Jun. 30, 2016; US20120115930, entitled “Compositions and their uses directed to hepcidin,” published May 10, 2012, the contents of each of which are incorporated herein by reference.

In some embodiments, the hepcidin neutralizing agent is an anticalin against hepcidin. Anticalin proteins are artificial proteins that are able to bind to antigens. Anticalin-proteins are engineered lipocalins, endogenous low-molecular weight human proteins typically found in blood plasma and other body fluids that naturally bind, store and transport a wide spectrum of molecules. In some embodiments, the lipocalin against hepcidin is a lipocalin as disclosed in U.S. Pat. No. 9,051,382B2, entitled “Human neutrophil gelatinase-associated lipocalin (hNGAL) muteins that bind hepcidin and nucleic acid encoding such”, issued Jun. 6, 2015; US20150369821, entitled “Novel lipocalin-mutein assays for measuring hepcidin concentration.”, published Dec. 14, 2015, the contents of each of which are incorporated herein by reference. In some embodiments, the anticalin against hepcidin is PRS-080.

In some embodiments, the hepcidin neutralizing agent is a PEGylated L-stereoisomer RNA aptamer that binds and neutralizes hepcidin. In some embodiments, the PEGylated L-stereoisomer RNA aptamer against hepcidin is a PEGylated L-stereoisomer RNA aptamer against hepcidin as described in U.S. Pat. No. 8,841,431B2, entitled “Hepcidin binding nucleic acids,” issued Sep. 23, 2-14; WO2012055573A1, entitled “Use of hepcidin binding nucleic acids for depletion of hepcidin from the body,” published May 3, 2012, the contents of each of which are incorporated herein by reference. In some embodiments, the PEGylated L-stereoisomer RNA aptamer against hepcidin is NOX-94. Examples of other molecules that specifically bind hepcidin include, without limitation, 12B9m, LS-B4534, lipocalin mutein, and hNGAL mutein. In some embodiments, other molecules that specifically bind hepcidin is a molecule as described in U.S. Pat. No. 8,629,250, entitled “Hepcidin, hepcidin antagonists and methods of use,” issued Jan. 14, 2014; U.S. Pat. No. 9,315,577, “Anti-hepcidin antibodies and methods of use,” issued Apr. 19, 2016; U.S. Pat. No. 9,051,382, entitled “Human neutrophil gelatinase-associated lipocalin (hNGAL) muteins that bind hepcidin and nucleic acid encoding such”, issued Jun. 9, 2015; U.S. Pat. No. 9,657,098 entitled “Anti-hepcidin antibodies and uses thereof”, issued May 23, 2017; U.S. Pat. No. 9,610,356 entitled “Methods for preventing or treating disorders by increasing bioavailability of iron and related pharmaceutical formulation”, issued Apr. 4, 2017; U.S. Pat. No. 8,530,619 entitled “Identification of the hepcidin binding site on ferroportin”, issued Sep. 10, 2013; US20150291675, entitled “Human neutrophil gelatinase-associated lipocalin (hngal) muteins that bind hepcidin and nucleic acid encoding such”, published Oct. 15, 2015, US20180057812, entitled “Hepcidin antagonists for use in the treatment of inflammation”, published Mar. 1, 2018; the contents of each of which are incorporated herein by reference). In some embodiments, the hepcidin neutralizing agent is a hepcidin neutralizing agent as described in U.S. Pat. No. 7,820,163B2, entitled “Anti-hepcidin antibodies and uses thereof”, issued Oct. 26, 2010, U.S. Pat. No. 8,328,308B2, entitled “Fluid ejecting apparatus, fluid ejecting head control method in fluid ejecting apparatus, and driving waveform generating apparatus for fluid ejecting head”, issued Dec. 11, 2012, HN2010000752A, entitled “Anti-hepcidin antibodies and uses thereof”, published Aug. 7, 2012, U.S. Pat. No. 8,609,817B2, entitled “Anti-hepcidin-25 selective antibodies and uses thereof”, issued Dec. 17, 2013, WO2015/051135A2, entitled “Organic compositions to treat hepcidin-related diseases”, published Apr. 9, 2015, U.S. Pat. No. 8,841,431B2, entitled “Hepcidin Binding Nucleic Acids”, issued Sep. 23, 2014, US2014/057970, entitled “Use of Hepcidin Binding Nucleic Acids for Depletion of Hepcidin From the Body”, published Feb. 27, 2014, US2015/0369821A1, entitled “Novel lipocalin-mutein assays for measuring hepcidin concentration”, published Dec. 24, 2015, U.S. Pat. No. 9,051,382B2, entitled “Binding proteins for hepcidin”, issued Jun. 9, 2015, U.S. Pat. No. 9,610,356B2, entitled “Methods for preventing or treating disorders by increasing bioavailability of iron and related pharmaceutical formulation”, issued Apr. 4, 2017, U.S. Pat. No. 9,228,188B2, entitled “Compositions and Method for Inhibiting Hepcidin Antimicrobial Peptide (HAMP) or HAMP-Related Gene Expression”, issued Jan. 5, 2016, U.S. Pat. No. 9,315,577B2, entitled “Anti-hepcidin antibodies and methods of use”, issued Apr. 19 2016, U.S. Pat. No. 8,629,250B2, entitled “Hepcidin, hepcidin antagonists and methods of use”, issued Jan. 14, 2014, WO2018/128828A1, entitled “Novel hepcidin mimetics and uses thereof”, published Jul. 12, 2018, WO2018/165186A1, entitled “Assessment of chronic iron deficiency”, published Sep. 13, 2018, U.S. Pat. No. 7,411,048B2, entitled “Diagnostic method for diseases by screening for hepcidin in human or animal tissues, blood or body fluids and therapeutic uses therefor”, issued Aug. 12, 2008, U.S. Pat. No. 8,915,875B2, entitled “Adsorbents for the adsorption of hepcidin”, issued Dec. 23, 2014, CN101816674A, entitled “Hepcidin inhibitor and application thereof”, published Sep. 1, 2010, U.S. Pat. No. 9,657,098B2, entitled “Anti-hepcidin antibodies and uses thereof”, issued May 23, 2017, U.S. Ser. No. 10/323,088B2, entitled “Humanized anti-hepcidin antibodies and uses thereof”, issued Jun. 18, 2019, U.S. Pat. No. 4,628,027A, entitled “Vitro diagnostic methods using monoclonal antibodies against connective tissue proteins”, issued Dec. 9, 1986, JP2019147772A, entitled “Hepcidin expression inhibitor, and food and drink for improvement and/or prevention of iron-deficiency anemia”, published Sep. 5, 2019, of EP2335708B1, entitled “Sulphated glycosaminoglycans, including heparin and derivatives thereof, for use in inhibiting the expression of hepcidin and for the therapeutic treatment of anaemia with high levels of hepcidin”, issued Oct. 9, 2013, US2016/0122409A1, entitled “Erythroferrone and erfe polypeptides and methods of regulating iron metabolism”, published May 5, 2016, US20120214803A1, entitled “Novel Sulfonaminoquinoline Hepcidin Antagonists”, published Aug. 23, 2012, WO2011/023722A1, entitled “Novel quinoxalinone hepcidin antagonists”, published Mar. 3, 2011, WO2011/029832A1, entitled “Novel thiazol and oxazol hepcidine antagonists”, published Mar. 17, 2011, US2012/0196853A1, entitled “Novel Quinoline-Hepcidine Antagonists”, published Aug. 2, 2012, US2012/0214798A1, entitled “Novel Ethanediamone Hepcidine Antagonists”, published Aug. 23, 2012, US2012/0202806A1, entitled “Novel Pyrimidine-And Triazine-Hepcidine Antagonists”, published Aug. 9, 2012, CN103655542B, entitled “Ampelopsin suppresses the application in the preparation of ferrum tune element expression in preparation”, published Apr. 13, 2016, the entire contents of each of which are incorporated herein by reference.

In some embodiments, the hepcidin inhibitor is a molecule that specifically binds ferroportin (e.g., an antibody, an anticalin, or an aptamer). In some embodiments, the molecule that specifically binds ferroportin is LY2928057. Molecules that bind ferroportin to inhibit hepcidin binding without affecting ferroportin activity have been described (see also U.S. Pat. Nos. 8,183,346, and 9,175,078, WO2010065496A1, the entire contents of each of which are incorporated herein by reference). In some embodiments, the hepcidin inhibitor is a chemical modifier compound that modifies hepcidin or ferroportin to inhibit the hepcidin-ferroportin binding interaction. For example, in some embodiments, the hepcidin inhibitor is fursultiamine (see, e.g., Fung and Nemeth. Haematologica. 2013 November; 98(11):1667-76).

iii. JAK/STAT Signaling Antagonists

In some embodiments, the hepcidin antagonist binds to and inhibits a molecule involved in the JAK-STAT signaling pathways. Accordingly, in some embodiments, the hepcidin antagonist is a JAK-STAT signaling pathway inhibitor. Examples such antagonists include, without limitation, IL-6, IL-6 receptors, JAK1/2, and STAT3. In some embodiments, the JAK-STAT signaling pathway inhibitor is a JAK inhibitor or a STAT inhibitor. In some embodiments, the JAK inhibitor is selective for one or both of subtypes JAK1 and JAK2 (e.g., a JAK1/2 inhibitor). In some embodiments, the STAT inhibitor is a STAT3 inhibitor.

The JAK-STAT3 signaling pathway is activated by the inflammatory cytokine IL-6. The binding of IL-6 to an IL-6 receptor (IL-6R) triggers receptor dimerization on hepatocytes, which leads to activation of the JAK-STAT3 signaling pathway (FIG. 2). Accordingly, in some embodiments, the JAK-STAT signaling pathway inhibitor is an IL-6 antagonist, which antagonizes hepcidin function by binding IL-6 and/or an IL-6 receptor to inhibit activation of the JAK-STAT3 signaling pathway. In some embodiments, the IL-6 antagonist is selected from the group consisting of Infliximab, Curcumin, 3,3′-Diindolyl-methane, Tocilizumab, and Siltuximab. In some embodiments, the IL-6 and IL-6R inhibitor is an anti-IL6 or IL-6R inhibitor as described in, for example, in US20170029499A1, entitled “Methods for treating hepcidin-mediated disorders”, published Feb. 2, 2017, WO2008144757A1, entitled “Novel rabbit antibody humanization methods and humanized rabbit antibodies”, published Nov. 27, 2008, US20090104187A1, entitled “Novel Rabbit Antibody Humanization Methods and Humanized Rabbit Antibodies”, published Apr. 23, 2009, WO2010065077A2, entitled “Antagonists of it-6 to prevent or treat thrombosis”, published Jun. 10, 2010, WO2011066369A2, entitled “Antagonists of it-6 to raise albumin and/or lower crp”, published Jun. 3, 2011, U.S. Pat. No. 9,701,747B2, entitled “Antagonists of il-6 to raise albumin and/or lower crp”, issued Jul. 11, 2017, U.S. Pat. No. 8,420,089B2, entitled “Antagonists of it-6 to raise albumin and/or lower crp”, issued Apr. 16, 2013, U.S. Pat. No. 9,265,825B2, entitled “Antagonists of IL-6 to raise albumin and/or lower crp”, issued Feb. 23, 2016, U.S. Pat. No. 8,277,804B2, entitled “Antagonists of it-6 to prevent or treat thrombosis”, issued Oct. 2, 2012, U.S. Pat. No. 9,085,615B2, entitled “Antibodies to it-6 and use thereof”, issued Jul. 21, 2015, WO2011066371A2, entitled “Antibodies to it-6 and use thereof”, published Jun. 3, 2011, U.S. Pat. No. 8,323,649B2, entitled “Antibodies to it-6 and use thereof”, issued Dec. 4, 2012, U.S. Pat. No. 9,452,227B2, entitled “Antibodies to IL-6 and use thereof”, issued Sep. 27, 2016, WO2010065079A2, entitled “Antibodies to it-6 and use thereof”, published Jun. 10, 2010, U.S. Pat. No. 9,724,410B2, entitled “Antagonists of it-6 to prevent or treat cachexia, weakness, fatigue and/or fever”, issued Aug. 8, 2017, US20090238825A1, entitled “Novel rabbit antibody humanization methods and humanized rabbit antibodies”, published Sep. 24, 2009, U.S. Pat. No. 9,993,480B2, entitled “mTOR/JAK INHIBITOR COMBINATION THERAPY”, issued Jun. 12, 2018, US20170029499A1, entitled “Methods for treating hepcidin-mediated disorders”, published Feb. 2, 2017, US20190241650A1, entitled “Methods for treating it-6 mediated inflammation without immunosuppression”, published Aug. 8, 2019, the entire contents of each of which are incorporated herein by reference.

In some embodiments, the JAK-STAT antagonist is a selective JAK1 inhibitor (e.g., as determined by the kinase potent assay described herein). In some embodiments, the JAK-STAT antagonist is a JAK2 inhibitor (e.g., as determined by the kinase potent assay described herein). In some embodiments, the JAK-STAT antagonist is not active against ACVR1/ALK2. In some embodiments, a JAK-STAT antagonist is ruxolitinib, fedratinib, pacritinib, baricitinib, tofacitinib, oclacitinib, NSC13626. In some embodiments, the JAK/STAT antagonist is GS-0387 or CYT-387.

In some embodiments, the JAK/STAT antagonist is a selective JAK1/JAK2 inhibitor. In some embodiments, the selective JAK1/JAK2 inhibitor is ruxolitinib. Suitable JAK1/JAK2 inhibitor for use in treating ACD are described in, e.g., U.S. Pat. No. 7,598,257, entitled “Heteroaryl substituted pyrrolo[2,3-b]pyridines and pyrrolo[2,3-b]pyrimidines as janus kinase inhibitors,” issued Oct. 6, 2009; U.S. Pat. No. 8,415,362, entitled “Pyrazolyl substituted pyrrolo[2,3-b]pyrimidines as Janus kinase inhibitors,” issued Apr. 9, 2013; U.S. Pat. No. 8,722,693, entitled “Salts of the Janus kinase inhibitor (R)-3-(4-(7H-pyrrolo[2,3-d]pyrimidin-4-yl)-1H-pyrazol-1-yl)-3-cyclopentylpropanenitrile,” issued May 13, 2014; U.S. Pat. No. 8,822,481, entitled “Salts of the janus kinase inhibitor (R)-3-(4-(7H-pyrrolo[2,3-d]pyrimidin-4-yl)-1H-pyrazol-1-yl)-3-cyclopentylpropanenitrile,” issued Sep. 2, 2014; U.S. Pat. No. 8,829,013, entitled “Salts of the Janus kinase inhibitor (R)-3-(4-(7H-pyrrolo[2,3-D]pyrimidin-4-yl)-1H-pyrazol-1-yl)-3-cyclopentylpropanenitrile,” issued Sep. 9, 2014; U.S. Pat. No. 9,079,912, entitled “Salts of the janus kinase inhibitor (R)-3-(4-(7H-pyrrolo[2,3-d]pyrimidin-4-yl)-1H-pyrazol-1-yl)-3-cyclopentylpropanenitrile,” issued Sep. 2, 2014; U.S. Pat. No. 9,814,722, entitled “Heteroaryl substituted pyrrolo[2,3-B] pyridines and pyrrolo[2,3-B] pyrimidines as janus kinase inhibitors,” issued Nov. 14, 2017; and U.S. Ser. No. 10/016,429B2, entitled “Salts of the janus kinase inhibitor (R)-3-(4-(7H-pyrrolo[2,3-D]pyrimidin-4-yl)-1H-pyrazol-1-yl)-3-cyclopentylpropanenitrile,” issued Jul. 10, 2018, the contents of each of which are incorporated herein by reference. In some embodiments, the JAK1/JAK2 inhibitor is ruxolitinib (RUX).

In some embodiments, the JAK-STAT inhibitor is a selective JAK2 inhibitor. Suitable JAK2 inhibitor for use in treating ACD are described in, for example, U.S. Pat. No. 7,528,143, entitled “Bi-aryl meta-pyrimidine inhibitors of kinases,” issued May 5, 2009; U.S. Pat. No. 7,825,246, entitled “Bi-aryl meta-pyrimidine inhibitors of kinases,” issued Nov. 2, 2010; U.S. Pat. No. 8,138,199, entitled “Use of bi-aryl meta-pyrimidine inhibitors of kinases,” issued Mar. 20, 2012; the entire contents of each of which are incorporated herein by reference. In some embodiments, the selective JAK2 inhibitor is fedratinib.

In some embodiments, the JAK-STAT inhibitor is not a selective JAK inhibitor. In some embodiments, the JAK-STAT inhibitor is an inhibitor of JAK1/JAK2, and ACVRI (also known as ALK2) (e.g., momelotinib). Suitable JAK1/JAK2 and ACVRI (ALK2) inhibitor for use in methods provided herein are described, for example, in U.S. Pat. No. 8,486,941B2, entitled “Phenyl amino pyrimidine compounds and uses thereof,” issued Jul. 16, 2013; U.S. Ser. No. 10/245,268B2, entitled “Momelotinib for treating of acvrl-mediated diseases,” issued Apr. 2, 2019, the entire contents of each of which are incorporated herein by reference. In some embodiments, the non-selective JAK-STAT inhibitor is Momelotinib (see, e.g., ASSHOFF MALTE ET AL: “The Jak1/Jak2 Inhibitor Momelotinib Inhibits Alk2, Decreases Hepcidin Production and Ameliorates Anemia of Chronic Disease (ACD) in Rodents”, BLOOD, vol. 126, no. 23, December 2015 (2015-12-01)).

In some embodiments, the JAK inhibitor is a JAK inhibitor as described in U.S. Pat. No. 8,202,881B2, entitled “JAK2 inhibitors and their use for the treatment of myeloproliferative diseases and cancer”, issued Jun. 19, 2020, U.S. Pat. No. 8,193,189 B2, entitled “Quinoxaline derivatives as tyrosine kinase activity inhibitors”, issued Jun. 5, 2012, U.S. Pat. No. 8,629,168 B2, entitled “Benzoxazoles and oxazolopyridines being useful as janus kinases inhibitors”, issued Jan. 14, 2014, US2014/073643, entitled “Treatment of jak2-mediated conditions”, published Mar. 13, 2014, U.S. Pat. No. 9,469,613 B2, entitled “(N-(cyanomethyl)-4-(2-(4-morpholinophenylamino)pyrimidin-4-yl)benzamide”, issued Oct. 18, 2016, WO2018/096525 A2, entitled “Heteroaryl compounds and uses thereof”, published May 31, 2018, U.S. Pat. No. 8,716,303B2, entitled “N-(hetero)aryl-pyrrolidine derivatives of pyrazol-4-yl-pyrrolo[2,3-d]pyrimidines and pyrrol-3-yl-pyrrolo[2,3-d]pyrimidines as janus kinase inhibitors”, issued May 6, 2014, U.S. Pat. No. 7,834,022B2, entitled “METABOLITES OF THE JANUS KINASE INHIBITOR (R)-3-(4-(7H-PYRROLO[2,3-d]PYRIMIDIN-4-YL)-1H-PYRAZOL-1-YL)-3-CYCLOPENTYLPROPANENITRILE”, issued Nov. 16, 2010, US20070149506A1, entitled “Azepine inhibitors of Janus kinases”, published Jun. 28, 2007, U.S. Pat. No. 8,513,270B2, entitled “Substituted heterocycles as janus kinase inhibitors”, issued Aug. 20, 2013, U.S. Pat. No. 9,359,358B2, entitled “Cyclohexyl Azetidine Derivatives as JAK Inhibitors”, issued Jun. 7, 2016, U.S. Pat. No. 8,486,902B2, entitled “HYDROXYL, KETO, AND GLUCURONIDE DERIVATIVES OF 3-(4-(7H-PYRROLO[2,3-d] PYRIMIDIN-4-YL)-1H-PYRAZOL-1-YL)-3-CYCLOPENTYLPROPANENITRILE”, issued Jul. 16, 2013, U.S. Pat. No. 9,358,229B2, entitled “JAK PI3K/mTOR COMBINATION THERAPY”, issued Jun. 7, 2016, U.S. Pat. No. 8,933,085B2, entitled “Cyclobutyl substituted pyrrolopyridine and pyrrolopyrimidine derivatives as jak inhibitors”, issued Jan. 13, 2015, U.S. Pat. No. 8,765,727B2, entitled “Macrocyclic compounds and their use as kinase inhibitors”, issued Jul. 1, 2014, U.S. Pat. No. 9,034,884B2, entitled “Heterocyclic-substituted pyrrolopyridines and pyrrolopyrimidines as jak inhibitors”, issued May 19, 2015, US20080312259A1, entitled “SALTS OF THE JANUS KINASE INHIBITOR (R)-3-(4-(7H-PYRROLO[2,3-d]PYRIMIDIN-4-YL)-1H-PYRAZOL-1-YL)-3-CYCLOPENTYLPROPANENITRILE”, published Dec. 18, 2008, US20150246046A1, entitled “Jak1 inhibitors for the treatment of myelodysplastic syndromes”, published Sep. 3, 2015, U.S. Pat. No. 8,158,616B2, entitled “Azetidine and cyclobutane derivatives as jak inhibitors”, issued Apr. 17, 2012, U.S. Pat. No. 7,335,667B2, entitled “Pyrrolo[2,3-b]pyridin-4-yl-amines and pyrrolo[2,3-b]pyrimidin-4-yl-amines as janus kinase inhibitors”, issued Feb. 26, 2008, U.S. Ser. No. 10/064,866B2, entitled “Treatment of b-cell malignancies by a combination jak and pi3k inhibitors”, issued Sep. 4, 2018, US20110207754A1, entitled “Cyclobutane and methylcyclobutane derivatives as janus kinase inhibitors”, published Aug. 25, 2011, U.S. Pat. No. 9,193,733B2, entitled “Piperidinylcyclobutyl substituted pyrrolopyridine and pyrrolopyrimidine derivatives as jak inhibitors”, issued Nov. 24, 2015, U.S. Pat. No. 9,382,231B2, entitled “Bipyrazole derivatives as jak inhibitors”, issued Jul. 5, 2016, U.S. Pat. No. 8,691,807B2, entitled “Azetidinyl phenyl, pyridyl or pyrazinyl carboxamide derivatives as jak inhibitors”, issued Apr. 8, 2014, U.S. Pat. No. 9,181,271B2, entitled “Tricyclic fused thiophene derivatives as jak inhibitors”, issued Nov. 10, 2015, U.S. Pat. No. 8,604,043B2, entitled “3-[4-(7h-pyrrolo[2,3-d]pyrimidin-4-yl)-1h-pyrazol-1-yl]octane- or heptane-nitrile as jak inhibitors”, issued Dec. 10, 2013, U.S. Pat. No. 9,249,145B2, entitled “HETEROCYCLIC DERIVATIVES OF PYRAZOL-4-YL-PYRROLO[2,3-d]PYRIMIDINES AS JANUS KINASE INHIBITORS”, issued Feb. 2, 2016, U.S. Pat. No. 8,765,734B2, entitled “Piperidin-4-yl azetidine derivatives as jak1 inhibitors”, issued Jul. 1, 2014, US20060106020A1, entitled “Tetracyclic inhibitors of Janus kinases”, published May 18, 2006, U.S. Pat. No. 9,993,480B2, entitled “mTOR/JAK INHIBITOR COMBINATION THERAPY”, issued Jun. 12, 2018, U.S. Pat. No. 8,309,718B2, entitled “4-pyrazolyl-n-arylpyrimidin-2-amines and 4-pyrazolyl-n-heteroarylpyrimidin-2-amines as janus kinase inhibitors”, issued Nov. 13, 2012, US20060153852A1, entitled “Novel human Jak2 kinase”, published Jul. 13, 2006, U.S. Pat. No. 8,871,753B2, entitled “Macrocyclic compounds and their use as kinase inhibitors”, issued Oct. 28, 2014, US20080287475A1, entitled “4-(3-Aminopyrazole) Pyrimidine Derivatives for Use as Tyrosine Kinase Inhibitors in the Treatment of Cancer”, published Nov. 20, 2008, US20090062302A1, entitled “Jak2 Tyrosine Kinase Inhibition”, published Mar. 5, 2009, U.S. Pat. No. 8,648,069B2, entitled “5-substituted indazoles as kinase inhibitors”, issued Feb. 11, 2014, WO2017196261A1, entitled “Jak and hdac dual-inhibitor compounds”, published Nov. 16, 2017, U.S. Pat. No. 9,949,971B2, entitled “Therapeutic Combinations of a BTK Inhibitor, a PI3K Inhibitor and/or a JAK-2 Inhibitor”, issued Apr. 24, 2018, US20170224819A1, entitled “Therapeutic Combinations of a BTK Inhibitor, a PI3K Inhibitor, a JAK-2 Inhibitor, and/or a CDK 4/6 Inhibitor”, published Aug. 10, 2017, US20170239351A1, entitled “Therapeutic Combinations of a BTK Inhibitor, a PI3K Inhibitor, a JAK-2 Inhibitor, a PD-1 Inhibitor, and/or a PD-L1 Inhibitor”, published Aug. 24, 2017, US20180317602A1, entitled “Combinations”, published Nov. 8, 2018, US20190135807A1, entitled “Pyrazolyl pyrrolo[2,3-b]pyrmidine-5-carboxylate analogs and methods of making the same”, published May 9, 2019, US20130089512A1, entitled “Heteroaryl imidazolone derivatives as jak inhibitors”, published Apr. 11, 2013, U.S. Pat. No. 9,206,183B2, entitled “Pyrazole derivatives as jak inhibitors”, issued Dec. 8, 2015, U.S. Pat. No. 9,034,311B2, entitled “Pyridin-2(1h)-one derivatives as jak inhibitors”, issued May 19, 2015, US20140170110A1, entitled “Pyridin-2(1h)-one derivatives useful as medicaments for the treatment of myeloproliferative disorders, transplant rejection, immune-mediated and inflammatory diseases”, published Jun. 19, 2014, WO2015091531A1, entitled “Imidazolopyrimidin-2-yl derivatives as jak inhibitors”, published Jun. 25, 2015, US20130216498A1, entitled “Imidazopyridine derivatives as jak inhibitors”, published Aug. 22, 2013, U.S. Pat. No. 9,133,200B2, entitled, “Imidazo [1,2-b] pyridazine and imidazo [4,5-b] pyridine derivatives as jak inhibitors”, issued Sep. 15, 2015, U.S. Pat. No. 8,349,851B2, entitled “JAK kinase modulating compounds and methods of use thereof”, issued Jan. 8, 2013, U.S. Pat. No. 9,206,188B2, entitled “Substituted pyrrolo[2,3-b]pyridines as ITK and JAK inhibitors”, issued Dec. 8, 2015, WO2010/020810 A1, entitled “2-(imidaz0lylamin0)-pyridine derivatives and their use as jak kinase inhibitors”, published Feb. 25, 2010, US2010/324040 A1, entitled “9-(pyrazol-3-yl)-9h-purine-2-amine and 3-(pyrazol-3-yl)-3h-imidazo[4,5-b]pyridin-5-amine derivatives and their use for the treatment of cancer”, published Jul. 16, 2013, WO2009/027736 A3, entitled “2,4 diaminopyrimid′lnes for the treatment of myeloproliferative disorders and cancer”, published Mar. 5, 2009, US2011/201628 A1, entitled “Heterocyclic jak kinase inhibitors”, published Aug. 18, 2011, WO2008/117050 A1, entitled “Pyrazolyl-amino-substituted pyrazines and their use for the treatment of cancer”, published Oct. 2, 2008, US2010/204246 A1, entitled “5-aminopyrazol-3-yl-3h-imidazo (4,5-b) pyridine derivatives and their use for the treatment of cancer”, published Aug. 12, 2010, WO2008/132502 A1, entitled “Pyrazolyl-amino-substituted pyrimidines and their use for the treatment of cancer”, published Nov. 6, 2008, WO2009/016410 A3, entitled “Chemical compounds 831”, published Feb. 5, 2009, WO2009/095712 A2, entitled “4-(3-amin0pyraz0le) pyrimidine derivatives for use as jak kinase inhibitors in the treatment of cancer”, published Aug. 6, 2009, U.S. Pat. No. 8,440,679B2, entitled “Bicyclic compounds and their uses as dual c-SRC/JAK inhibitors”, issued May 14, 2013, U.S. Pat. No. 9,035,074B2, entitled “Pyrrolo[2,3-D]pyrimidine derivatives”, issued May 16, 2015, WO2019057112 A1, entitled “2-substituted pyrazole amino-4-substituted amino-5-pyrimidine formamide compound, composition, and application thereof”, published Mar. 28, 2019, U.S. Pat. No. 8,815,840B2, entitled “Carbazole and carboline kinase inhibitors”, issued Aug. 26, 2014, U.S. Pat. No. 8,957,065B2, entitled “Fused pyrimidine derivatives for inhibition of tyrosine kinase activity”, issued Feb. 17, 2015, WO2014020531 A1, entitled “Imidazo[1,2-b]pyridazin-6-amine derivatives as kinase jak-2 inhibitors”, published Feb. 6, 2014, WO2015118434 A1, entitled “PYRAZOLO[1,5-a]PYRIMIDINE DERIVATIVES AS KINASE JAK-2 INHIBITORS”, published Aug. 13, 2015, US2008021013 A1, entitled “JAK inhibitors for treatment of myeloproliferative disorders”, published Jan. 24, 2008, U.S. Pat. No. 8,937,065B2, entitled “Compositions and methods for modulating a kinase”, issued Jan. 20, 2015, US2015/197525 A1, entitled “Deuterated derivatives of ruxolitinib”, published Jul. 16, 2015, U.S. Pat. No. 9,540,367B2, entitled “Deuterated baricitinib”, issued Jan. 10, 2017, U.S. Pat. No. 9,518,027B2, entitled “Deuterated momelotinib”, issued Dec. 13, 2016, U.S. Pat. No. 8,354,408B2, entitled “N-containing heterocyclic compounds”, issued Jan. 15, 2013, WO2020097396 A1, entitled “Benzimidazole derivatives and aza-benzimidazole derivatives as janus kinase 2 inhibitors and uses thereof”, published May 14, 2020, WO2020097398 A1, entitled “Benzothiazole derivatives and 7-aza-benzothiazole derivatives as janus kinase 2 inhibitors and uses thereof”, published May 14, 2020, WO202009740 A3, entitled “Compositions and methods of use thereof for treatment of metabolic diseases and related disorders”, published Jan. 9, 2020, U.S. Ser. No. 10/111,897B2, entitled “Compositions and methods for treating cancer with JAK2 activity”, issued Oct. 30, 2018, U.S. Pat. No. 8,440,663B2, entitled “4-aryl-2-amino-pyrimidines or 4-aryl-2-aminoalkyl-pyrimidines as JAK-2 modulators and methods of use”, issued May 14, 2013, U.S. Pat. No. 8,088,767B2, entitled “JAK-2 modulators and methods of use”, issued Jan. 3, 2012, WO2009017838 A2, entitled “Combinations of jak-2 inhibitors and other agents”, published Feb. 5, 2009, US2010/035875 A1, entitled “Triazolopyridine jak inhibitor compounds and methods”, published Feb. 11, 2010, US2010/048557 A1, entitled “Triazolopyridine JAK Inhibitor Compounds and Methods”, published Feb. 25, 2010, U.S. Ser. No. 10/307,426B2, entitled “Therapeutic compounds and compositions, and methods of use thereof”, issued Jun. 4, 2019, U.S. Pat. No. 8,637,526B2, entitled “Pyrazolopyrimidine JAK inhibitor compounds and methods”, issued Jan. 28, 2014, U.S. Pat. No. 8,999,998B2, entitled “Pyrazolopyrimidine JAK inhibitor compounds and methods”, issued Apr. 7, 2015, US2019/040068 A1, entitled “Pyrrolopyrimidine five-membered azacyclic derivative and application thereof”, published Feb. 7, 2019, US2019/328857 A1, entitled “Calr and jak2 vaccine compositions”, published Oct. 31, 2019, WO11153586 A1, entitled “Kinase inhibitors”, published Dec. 15, 2011, U.S. Ser. No. 10/294,226B2, entitled “Small molecule inhibitors of the JAK family of kinases”, issued May 21, 2019, US2019/322665 A1, entitled “Imidazopyrrolopyridine as inhibitors of the jak family of kinases”, published Oct. 24, 2019, U.S. Pat. No. 8,901,145B2, entitled “Aminopyrimidine kinase inhibitors”, issued Dec. 2, 2014, U.S. Pat. No. 8,563,539B2, entitled “Aminopyrimidine kinase inhibitors”, issued Oct. 22, 2013, US2018/002328 A1, entitled “Substituted imidazo[1, 2-a]pyridin-2-ylamine compounds, and pharmaceutical compositions and methods of use thereof”, published Jan. 4, 2018, WO2017/143014 A1, entitled “Jak inhibitors and uses thereof”, published Aug. 24, 2017, WO2019/107943 A1, entitled “Jak inhibitor compound and preparation method therefor”, published Jun. 6, 2019, WO2008/047831 A1, entitled “JAK inhibitor”, published Feb. 25, 2010, WO2010/141062 A1, entitled “Inhibitors of constitutively active janus kinases and uses thereof”, published Dec. 9, 2010, U.S. Pat. No. 8,278,335B2, entitled “Inhibitors of Janus kinases”, issued Oct. 2, 2012, U.S. Pat. No. 8,349,865B2, entitled “Inhibitors of janus kinases”, issued Jan. 8, 2013, U.S. Pat. No. 8,344,144B2, entitled “Inhibitors of Janus kinases”, issued Jan. 1, 2013, U.S. Pat. No. 8,420,695B2, entitled “Inhibitors of janus kinases”, issued Apr. 16, 2013, U.S. Pat. No. 8,367,706B2, entitled “Inhibitors of janus kinases”, issued Feb. 5, 2013, U.S. Pat. No. 8,431,569B2, entitled “Inhibitors of janus kinases”, issued Apr. 30, 2014, U.S. Pat. No. 8,415,346B2, entitled “Inhibitors of Janus kinases”, issued Apr. 9, 2013, U.S. Pat. No. 8,183,245B2, entitled “Pyrazine substituted pyrrolopyridines as inhibitors of JAK and PDK1”, issued May 22, 2012, U.S. Pat. No. 7,763,634B2, entitled “Inhibitors of janus kinases”, issued Jul. 27, 2010, U.S. Pat. No. 9,283,224B2, entitled “Substituted pyrimidinyl-pyrroles active as kinase inhibitors”, issued Mar. 15, 2016, U.S. Pat. No. 8,912,200B2, entitled “Alkynyl substituted pyrimidinyl-pyrroles active as kinases inhibitors”, issued Dec. 16, 2014, U.S. Pat. No. 9,688,661B2, entitled “Substituted pyrroles active as kinases inhibitors”, issued Jun. 27, 2017, U.S. Pat. No. 8,673,891B2, entitled “Aminopyrazine derivative and medicine”, issued Mar. 18, 2014, U.S. Pat. No. 8,629,168B2, entitled “Benzoxazoles and oxazolopyridines being useful as janus kinases inhibitors”, issued Jan. 14, 2014, U.S. Pat. No. 8,501,735B2, entitled “N-containing heteroaryl derivatives as JAK3 kinase inhibitors”, issued Aug. 6, 2013, WO2010/072823A1, entitled “PYRAZOLE[1,5a]PYRIDINE DERIVATIVES”, published Jul. 1, 2010, U.S. Pat. No. 8,633,206B2, entitled “Pyrrolo[2,3-D]pyrimidine compounds”, issued Jan. 21, 2014, U.S. Pat. No. 9,617,258B2, entitled “Pyrrolo[2,3-d]pyrimidinyl, pyrrolo[2,3-b]pyrazinyl and pyrrolo[2,3-d]pyridinyl acrylamides”, issued Apr. 11, 2017, WO2009/017954A1, entitled “Inhibitors of jak2 kinase”, published Feb. 5, 2009, U.S. Pat. No. 8,258,144B2, entitled “Inhibitors of protein kinases”, issued Sep. 4, 2012, U.S. Pat. No. 9,676,756B2, entitled “Substituted pyrimidinyl kinase inhibitors”, issued Jun. 13, 2017, U.S. Pat. No. 9,533,986B2, entitled “Bicyclic dihydropyridone kinase inhibitors”, issued Jan. 3, 2017, U.S. Pat. No. 9,469,654B2, entitled “Bicyclic oxa-lactam kinase inhibitors”, issued Oct. 18, 2016, U.S. Pat. No. 8,138,339B2, entitled “Inhibitors of protein kinases”, issued Mar. 20, 2012, U.S. Pat. No. 8,309,566B2, entitled “Pyrimidine-2-amine compounds and their use as inhibitors of JAK kinases”, issued Nov. 13, 2012, U.S. Pat. No. 8,846,908B2, entitled “Tricyclic carbamate JAK inhibitors”, issued Sep. 30, 2014, U.S. Ser. No. 10/011,571B2, entitled “Preparation method for aromatic heterocyclic compound used as selective JAK3 and/or JAK1 kinase inhibitor and application of aromatic heterocyclic compound”, issued Jul. 3, 2018, US2020/071303 A1, entitled “Diphenylaminopyrimidine compound for inhibiting kinase activity”, published Mar. 5, 2020, U.S. Pat. No. 9,371,320B2, entitled “Heterocyclic compound”, issued Jun. 21, 2016, U.S. Pat. No. 9,637,483B2, entitled “Heterocyclic compound”, issued May 2, 2017, WO2019/069844, entitled “Heterocyclic compound”, published Apr. 11, 2019, WO2009/046416 A1, entitled “Anilinopyrimidines as jak kinase inhibitors”, published May 9, 2009, WO2009/049028 entitled “Pyrrolopyrimidine compounds and their use as janus kinase modulators”, published Apr. 16, 2009, WO2009/055674 A1, entitled “Pyrrolopyrimidine alkynyl compounds and methods of making and using same”, published Apr. 30, 2009, U.S. Ser. No. 10/202,356B2, entitled “JAK2 and ALK2 inhibitors and methods for their use”, issued Feb. 12, 2019, US2019/169208 A1, entitled “Macrocycle kinase inhibitors”, published Jun. 6, 2019, US2011/243853 A1, entitled “Models of erythropoiesis”, published Oct. 6, 2011, U.S. Pat. No. 8,367,078B2, entitled “Kinase inhibitor compounds”, issued Feb. 5, 2013, US2015/306112 A1, entitled “Zhankuic acid A, a JAK2/3 tyrosine kinase inhibitor, and a potential therapeutic agent for hepatitis”, published Oct. 29, 2015, US2014/286964 A1, entitled “Methods for Identifying Janus Kinase (JAK) Modulators for Therapeutics”, published Sep. 25, 2014, U.S. Pat. No. 8,937,064B2, entitled “Pyrazolo[1,5-a]pyrimidines useful as JAK2 inhibitors”, issued Jan. 20, 2015, U.S. Pat. No. 7,767,816B2, entitled “Azaindoles useful as inhibitors of janus kinases”, issued Aug. 3, 2010, U.S. Pat. No. 8,580,802B2, entitled “Pyrrolo[2,3-D]pyrimidines as inhibitors of Janus kinases”, issued Nov. 12, 2013, U.S. Pat. No. 8,633,205B2, entitled “Substituted pyrrolo[2,3-d]pyrimidines as inhibitors of protein kinases”, issued Jan. 21, 2014, U.S. Pat. No. 8,741,912B2, entitled “Deazapurines useful as inhibitors of Janus kinases”, issued Jun. 3, 2014, US2010/160287 A1, entitled “Compounds useful as inhibitors of janus kinases”, published Jun. 24, 2010, U.S. Pat. No. 8,921,376B2, entitled “Pyrrolopyridines useful as inhibitors of protein kinase”, issued Dec. 30, 2014, US2014/073643 A1, entitled “Treatment of jak2-mediated conditions”, published Mar. 13, 2014, U.S. Pat. No. 8,809,359B2, entitled “Phenyl amino pyrimidine bicyclic compounds and uses thereof”, issued Aug. 19, 2014, the entire contents of each of which are incorporated herein by reference.

In some embodiments, the JAK/STAT inhibitor is a STAT inhibitor. STAT inhibitor have been previously described, e.g., U.S. Pat. No. 8,779,001B2, entitled “Stat3 inhibitors”, issued Jul. 15, 2014, WO2019/204427A1 entitled “Methods for measuring and stabilizing stat3 inhibitors”, published Oct. 24, 2019, U.S. Ser. No. 10/112,933B2, entitled “Methods and compositions for treatment of fibrosis”, issued Oct. 30, 2018, U.S. Pat. No. 9,650,399B2, entitled “Salicylic acid derivatives, pharmaceutically acceptable salt thereof, composition thereof and method of use thereof”, issued May 16, 2017, the entire contents of each of which are incorporated herein by reference.

iv. Chromatin Modulators

In some embodiments, the chromatin remodeling Bromodomain and Extra-Terminal (BET) proteins regulate genes that are involved in inflammation such as MYC, BCL-2, and NF-kB. In some embodiments, the NF-kB pathway downstream to BET is activated in ACD, e.g., via JAK-STAT signaling. BET proteins act as epigenetic readers, transmitting the signal carried by acetylated lysine residues on histones and transcribing it into various phenotypes. Dysregulated BET signaling is involved in a number of diseases including ACD. BET inhibitors have been described, see, e.g., US20170333406A1, which is incorporated herein by reference. In some embodiments, the BET inhibitor for use in the present disclosure is CPI-0610. CPI-0610 is a potent and selective small molecule designed to promote anti-tumor activity by selectively inhibiting the function of BET proteins to decrease the expression of abnormally expressed genes in cancer. BET proteins bind to acetylated histone lysine residues and function as co-activators of gene expression. They cooperate with the transcription factor NFκB to activate pro-inflammatory cytokine gene expression. CP-0610 downregulated pro-inflammatory cytokines in mouse models, and the combination of a BET inhibitor and ruxolitinib synergistically reduced splenomegaly, cytokine expression, bone marrow fibrosis, and the mutant allele burden. In some embodiments, the BET inhibitors suitable for use in the method described herein are BET inhibitors as described in US2019152949, entitled “Therapeutic compounds and uses thereof,” published May 23, 2019; U.S. Ser. No. 10/206,931B2, entitled “Therapeutic compounds and uses thereof,” issued Feb. 19, 2019; US2016317632, entitled “Use of cbp/ep300 bromodomain inhibitors for cancer immunotherapy,” published Nov. 3, 2016, US2017196878, entitled “Use of cbp/ep300 and bet inhibitors for treatment of cancer,” published Jul. 13, 2017, WO2019161162, entitled “P300/cbp hat inhibitors,” published Aug. 22, 2019, WO2020112086, entitled “Methods of treating myeloproliferative disorders,” published Jun. 4, 2020, WO2019161157, “entitled “P300/cbp hat inhibitors,” published Jun. 11, 2020, the entire contents of each of which are incorporated herein by reference.

v. Immunomodulatory Agents/Erythropoietin Stimulating Agent

In some embodiments, an immunomodulatory agent/erythropoietin stimulating agent provided herein for the treatment of anemia, e.g., as associated with chronic diseases. Such immunomodulatory agents/erythropoietin stimulating agent include, for example, corticosteroids, androgenic steroids, thalidomide, pomalidomide, lenalidomide and others. In some embodiments, the immunomodulatory agents are advantageous in that they have beneficial effects in reducing inflammation and in promoting erythropoiesis. Danazol, for example, is a steroid compound having hematopoietic stimulatory and immunomodulatory effects. For example, in some embodiments, danazol has antagonistic effects on glucocorticoid receptors, resulting in upregulating effects on erythropoiesis (see, e.g., Chai K Y, et al., Danazol: An Effective and Underutilized Treatment Option in Diamond-Blackfan Anaemia. Case Reports in Hematology. Volume 2019, Article ID 4684156.). Similarly, in some embodiments, glucocorticoids, such as prednisone, which promote erythropoiesis, may be useful for reducing inflammation, e.g., in the fibrotic marrow of MF patients (see, e.g., Amylon M D et al., Prednisone stimulation of erythropoiesis in leukemic children during remission. American Journal of Hematology. Volume 23, Issue 2, October 1986.) Other immunomodulatory agents/erythropoietin stimulating agents that affect erythropoiesis including thalidomide and derivatives or analogs thereof, such as lenalidomide, and pomalidomide. In some embodiments, erythropoietin (EPO) can be used in the methods described herein.

### III. METHODS OF USE

Aspects of the disclosure relate to compositions and methods for treating ACD and/or one or more conditions arising as a result of ACD in a subject.

In some aspects, the disclosure relates to compositions and methods for treating ACD in a subject. In some embodiments, a subject to be treated in accordance with the disclosure may be identified based on an appropriate diagnostic methodology, as described, for example, in Cullis, J O, Diagnosis and management of anaemia of chronic disease: current status British Journal of Haematology, Volume 154, Issue 3, August 2011 pages 289-300; and Madua A J and Ughasoro M D, Anaemia of Chronic Disease: An In-Depth Review Med Princ Pract. 2017 January; 26(1): 1-9, the contents of each of which are incorporated herein by reference. Typically, diagnosis involves an evaluation of signs and symptoms of the underlying chronic condition combined with an assessment of indicia of anemia and/or defects in iron metabolism, including, for example, through an analysis of complete blood count (CBC), serum iron, ferritin, transferrin, reticulocyte count, and other markers. In some embodiments, ACD may be present in subjects having microcytic or normocytic anemia who also have chronic conditions such as an infection, autoimmune disease, chronic kidney disease or cancer, e.g., myeloma.

In some embodiments, a subject in need of treatment in accordance with the disclosure may be identified based on a reduced EPO production. For example, under normal physiological conditions, levels of EPO are inversely correlated with hemoglobin levels and tissue oxygenation, but in chronic inflammatory conditions the EPO response is blunted, leading to inadequate levels of EPO for the degree of anemia, and this is thought to be mediated via inflammatory cytokines such as IL-1 and tumor necrosis factor-α (TNF-α). Accordingly, in some embodiments, a blunted EPO response may be diagnostic of ACD in a subject.

In some embodiments, a subject in need of treatment in accordance with the disclosure may be identified based on a reduced erythroid responsiveness. For example, ACD may be characterized by a reduced proliferation and differentiation of erythroid progenitor cells. It has been shown that macrophages from patients with ACD suppress colony formation in vitro due to inhibitory effects of inflammatory cytokines (e.g., interferon-y) on growth of erythroid burst-forming units (BFU-E) and erythroid colony-forming units (CFU-E), and that this effect could be overcome by addition of high concentrations of EPO to the culture systems. It has also been shown that bone marrow cultures from patients with active rheumatoid arthritis showed defective growth when compared to normal controls, and that there was an inverse correlation between colony growth and levels of TNF-α in the culture supernatant. Moreover, these effects were reversed both in vitro and in vivo following treatment with infliximab, an antibody against TNF-α. Accordingly, in some embodiments, a reduced erythroid responsiveness in a subject can be identified using these or similar such assays for evaluating erythroid responsiveness.

In some embodiments, a subject in need of treatment in accordance with the disclosure may be identified based on an anemic state that is mild to moderate and/or normochromic and normocytic (although anemia may become microcytic as disease progresses). In some embodiments, a subject is identified based on a low reticulocyte count, which may be indicative of the hypo-proliferative nature of ACD in the subject. Inflammation in a subject may be inferred from other features of the blood count, such as neutrophilia, monocytosis or thrombocytosis, and through measurement of non-specific inflammatory markers, such as C-reactive protein (CRP) or erythrocyte sedimentation rate (ESR).

In some embodiments, a subject in need of treatment in accordance with the disclosure may be identified based on the presence or absence of iron-deficiency anemia (IDA) in the subject. In some embodiments, a subject is identified by exclusion of IDA in the subject, although ACD and IDA can co-exist. Typically, serum iron and transferrin saturation are both decreased in ACD and iron deficiency, indicating limited iron supply to the erythron, but transferrin levels are increased in IDA, whereas in ACD they are normal or decreased. Accordingly, in some embodiments, the subject is identified by an assessment of iron stores in a biological sample of the subject, e.g., using a Perl's stained bone marrow aspirate.

In some embodiments, a subject is identified by determining a level of growth differentiation factor 15 (GDF15) in the subject. GDF15 is an erythropoiesis-derived hormone that is markedly increased in β-thalassemia and congenital dyserythropoietic anemia, and inhibits hepcidin expression, contributing to the iron overload seen in these anemias. Levels of GDF15 have been studied in patients with ACD, ACD/IDA and IDA. Subjects with both ACD and ACD/IDA showed significantly higher levels of GDF15 than patients with IDA, and GDF15 concentrations correlated with interleukin-1β, suggesting that inflammation induces GDF15 expression. Accordingly, in some embodiments, the subject is identified based on an increased level of GDF15 (e.g., increased as compared to a subject known to have IDA or a healthy subject).

In some embodiments, a subject is identified by determining a ratio of serum transferrin receptor (sTFR) to ferritin. The measurement of sTFR, the truncated fragment of the membrane receptor, has been described as an indicator for differentiating between ACD and IDA in a subject. The transferrin receptor is found on virtually all cells in the body, but is present at high levels on erythroid progenitors. sTFR levels increase in IDA as the availability of iron for erythropoiesis decreases, whereas in ACD levels may not differ from steady state because transferrin receptor expression is negatively affected by inflammatory cytokines. The ratio of sTFR to the log of the serum ferritin may be used in the diagnosis of ACD in a subject, and in some cases may be used for differentiating ACD from IDA. A ratio of less than 1 makes ACD likely, whereas ratios of greater than 2 suggest that iron stores are deficient, with or without ACD.

In some embodiments, a subject in need of treatment in accordance with the disclosure may be identified using red cell indices. For example, the reticulocyte hemoglobin content (CHr) and the percentage hypochromic red cells (% HYPO) can provide information about iron supply to the erythron, and may be useful in guiding the management of ACD. CHr is a measure of hemoglobin in the most recently formed erythrocytes, while the % HYPO indicates the percentage of cells with hemoglobin content of <280 g/l. The former gives a relatively acute evaluation of recent bone marrow activity (e.g., 48 hours), whereas the latter gives a time-averaged picture (e.g., 20-120 days). Similar indices can be reported by the Sysmex XE-2100 analyser (Sysmex, Mundelein, Ill., USA), which derives RET-Y (equivalent to CHr) and RBC-Y (equivalent to HYPO %). CHr has been shown to be a useful tool in the detection of early iron deficiency, as well as in monitoring early response to iron therapy.

Accordingly, in some embodiments, a subject in need of treatment in accordance with the disclosure has previously received therapeutic intervention for a hematologic disorder. In some embodiments, the subject has previously undergone a surgical procedure for treating one or more hematologic disorders. In some embodiments, the subject has previously undergone a splenectomy. In some embodiments, the subject has previously received a therapeutic agent for treating one or more hematologic disorders.

In some embodiments, a subject has previously received an erythropoietin stimulating agent. In some embodiments, the erythropoietin stimulating agent is selected from the group consisting of danazol, prednisone, thalidomide, lenalidomide, and pomalidomide.

In some embodiments, a subject has previously received a JAK-STAT pathway inhibitor. In some embodiments, the JAK-STAT pathway inhibitor is a JAK inhibitor or a STAT inhibitor. In some embodiments, the JAK inhibitor is selective for one or both of subtypes JAK1 and JAK2 (e.g., a JAK1/2 inhibitor). In some embodiments, the STAT inhibitor is a STAT3 inhibitor. In some embodiments, the JAK1/2 or STAT3 inhibitor is selected from the group consisting of ruxolitinib, momelotinib, pacritinib, INCB039110, AG490, and PpYLKTK.

In some embodiments, a subject has previously received a growth factor ligand trap. In some embodiments, the growth factor ligand trap is a transforming growth factor beta (TGF-β) ligand trap. In some embodiments, the TGF-β ligand trap is sotatercept or luspatercept. In some embodiments, a subject has previously received an anti-fibrotic agent. In some embodiments, the anti-fibrotic agent is PRM-151.

In some embodiments, a subject in need of treatment in accordance with the disclosure continues to receive a therapeutic treatment for a hematologic disorder. The disclosure therefore provides, in some aspects, compositions and methods for treating ACD and/or one or more conditions arising as a result of ACD by administering to a subject in need thereof a hepcidin antagonist (e.g., a hemojuvelin antagonist) in combination with one or more therapeutic treatments for a hematologic disorder.

In some aspects, the disclosure provides compositions and methods for treating a subject that is known to have, or is suspected of having, a hematologic disorder characterized by low systemic iron levels (e.g., anemia of chronic diseases). In some embodiments, the subject has a chronic disease and anemia associated with the chronic disease, as described elsewhere herein. In some embodiments, the anemia in the subject is addressed by erythrocyte-transfusion. In some embodiments, the subject is erythrocyte-transfusion dependent. “Transfusion dependent” may refer to a patient with an erythrocyte-transfusion-frequency of at least 2 units of packed red blood cells transfused per four week period averaged over the prior twelve weeks. The transfusion dependent patient may also have no consecutive four or six week period with an erythrocyte transfusion during the previous twelve or twenty-four weeks. In some embodiments, the subject is erythrocyte-transfusion independent. “Transfusion independent” may refer to a patient that is anemic (e.g. a Hgb level of no more than 11 g/dL, no more than 10 g/dL or no more than 9 g/dL). A patient may also be intermittently transfused, meaning a patient that is anemic and does not meet the criteria for either transfusion dependent or transfusion independent. In some embodiments, the subject has received multiple transfusions over a twelve week period. In some embodiments, the subject has received at least four RBC transfusions in a twelve week period. In some embodiments the subject has received at least one transfusion of two units of packed red blood cells in a four, six or eight week period and in some embodiments the subject has also received at least four, six or eight units of packed red blood cells transfused over a twelve week period. In some embodiments, the subject may have a reduced transfusion burden reduction in at least 10%, at least 20%, at least 30%, at least 40%, at least 50%, at least 60%, at least 70%, at least 80%, at least 90% or more. In some embodiments, the subject is transfusion independent (e.g., no rolling twelve week period for erythrocyte-transfusion). In some embodiments the subject is anemic (e.g., Hgb level less than 10 g/dL) and receives occasional transfusions but fewer than six units of packed red blood cells in the last twelve week period.

In some aspects, the disclosure relates to compositions and methods for treating ACD. Any of the anemic conditions described herein may be characterized with one or more of the hematological standard described herein. In some embodiments, the ACD may be characterized as a mild to moderate anemia or a severe anemia in accordance with appropriate diagnostic threshold parameters. For example, in some embodiments, the ACD is characterized based on a level of hemoglobin (Hgb), wherein the severity of the anemia increases with decreasing levels of Hgb. In some embodiments, mild to moderate anemia is associated with Hgb levels of at least 8 g/dL and less than the lower limit of normal (e.g., between about 8 and about 14 g/dL, between about 8 and about 12 g/dL, between about 8 and about 10 g/dL, between about 10 and about 14 g/dL, or between about 10 and about 12 g/dL). In some embodiments, severe anemia is associated with Hgb levels of about 8 g/dL or lower (e.g., between about 2 and about 8 g/dL, between about 4 and about 8 g/dL, or between about 6 and about 8 g/dL). In some embodiments, severe anemia is associated with erythrocyte-transfusion dependence. In some embodiments, severe anemia is associated with erythrocyte-transfusion independence resulting from a therapeutic intervention (e.g., therapeutic reversal of a transfusion dependent state), where a subject is dependent upon ongoing therapeutic treatment to maintain transfusion independence.

In some embodiments, the ACD is also characterized based on a level of ferritin. Ferritin is a blood protein that contains iron, the level of which indicates how much iron the body stores. In some embodiments, the subject has a serum ferritin level within or above normal. The normal range of ferritin is 24-336 μg/L for man, and 11-307 μg/L for women. In some embodiments, the subject has a serum ferritin level of more than 100 μg/L (e.g., between about 100 μg/L and about 110 μg/L, between about 100 μg/L and about 120 μg/L, between about 100 μg/L and about 130 μg/L, between about 100 μg/L and about 140 μg/L, between about 100 μg/L and about 150 μg/L, between about 110 μg/L and about 120 μg/L, between about 110 μg/L and about 130 μg/L, between about 110 μg/L and about 140 μg/L, between about 110 μg/L and about 150 μg/L, between about 120 μg/L and about 130 μg/L, between about 120 μg/L and about 140 μg/L, between about 120 μg/L and about 150 μg/L, between about 130 μg/L and about 140 μg/L, between about 130 μg/L and about 150 μg/L, between about 140 μg/L and about 150 μg/L), more than 150 μg/L (e.g., between about 150 μg/L and about 160 μg/L, between about 150 μg/L and about 170 μg/L, between about 150 μg/L and about 180 μg/L, between about 150 μg/L and about 190 μg/L, between about 150 μg/L and about 200 μg/L, between about 160 μg/L and about 170 μg/L, between about 160 μg/L and about 180 μg/L, between about 160 μg/L and about 190 μg/L, between about 160 μg/L and about 200 μg/L, between about 170 μg/L and about 180 μg/L, between about 170 μg/L and about 190 μg/L, between about 170 μg/L and about 200 μg/L, between about 180 μg/L and about 190 μg/L, between about 180 μg/L and about 200 μg/L, between about 190 μg/L and about 200 μg/L), more than 200 μg/L (e.g., between about 200 μg/L and about 210 μg/L, between about 200 μg/L and about 220 μg/L, between about 200 μg/L and about 230 μg/L, between about 200 μg/L and about 240 μg/L, between about 200 μg/L and about 250 μg/L, between about 210 μg/L and about 220 μg/L, between about 210 μg/L and about 230 μg/L, between about 210 μg/L and about 240 μg/L, between about 210 μg/L and about 250 μg/L, between about 220 μg/L and about 230 μg/L, between about 220 μg/L and about 240 μg/L, between about 220 μg/L and about 250 μg/L, between about 230 μg/L and about 240 μg/L, between about 230 μg/L and about 250 μg/L, between about 240 μg/L and about 250 μg/L), more than 250 μg/L (e.g., between about 250 μg/L and about 260 μg/L, between about 250 μg/L and about 270 μg/L, between about 250 μg/L and about 280 μg/L, between about 250 μg/L and about 290 μg/L, between about 250 μg/L and about 300 μg/L, between about 260 μg/L and about 270 μg/L, between about 260 μg/L and about 280 μg/L, between about 260 μg/L and about 290 μg/L, between about 260 μg/L and about 300 μg/L, between about 270 μg/L and about 280 μg/L, between about 270 μg/L and about 290 μg/L, between about 270 μg/L and about 300 μg/L, between about 280 μg/L and about 290 μg/L, between about 280 μg/L and about 300 μg/L, between about 290 μg/L and about 300 μg/L), more than 300 μg/L (e.g., between about 300 μg/L and about 310 μg/L, between about 300 μg/L and about 320 μg/L, between about 300 μg/L and about 330 μg/L, between about 300 μg/L and about 340 μg/L, between about 300 μg/L and about 350 μg/L, between about 310 μg/L and about 320 μg/L, between about 310 μg/L and about 330 μg/L, between about 310 μg/L and about 340 μg/L, between about 310 μg/L and about 350 μg/L, between about 320 μg/L and about 330 μg/L, between about 320 μg/L and about 340 μg/L, between about 320 μg/L and about 350 μg/L, between about 330 μg/L and about 340 μg/L, between about 330 μg/L and about 350 μg/L, between about 340 μg/L and about 350 μg/L), more than 350 μg/L (e.g., between about 350 μg/L and about 360 μg/L, between about 350 μg/L and about 370 μg/L, between about 350 μg/L and about 380 μg/L, between about 350 μg/L and about 390 μg/L, between about 350 μg/L and about 400 μg/L, between about 360 μg/L and about 370 μg/L, between about 360 μg/L and about 380 μg/L, between about 360 μg/L and about 390 μg/L, between about 360 μg/L and about 400 μg/L, between about 370 μg/L and about 380 μg/L, between about 370 μg/L and about 390 μg/L, between about 370 μg/L and about 400 μg/L, between about 380 μg/L and about 390 μg/L, between about 380 μg/L and about 400 μg/L, between about 390 μg/L and about 400 μg/L), more than 400 μg/L (e.g., between about 400 μg/L and about 410 μg/L, between about 400 μg/L and about 420 μg/L, between about 400 μg/L and about 430 μg/L, between about 400 μg/L and about 440 μg/L, between about 400 μg/L and about 450 μg/L, between about 410 μg/L and about 420 μg/L, between about 410 μg/L and about 430 μg/L, between about 410 μg/L and about 440 μg/L, between about 410 μg/L and about 450 μg/L, between about 420 μg/L and about 430 μg/L, between about 420 μg/L and about 440 μg/L, between about 420 μg/L and about 450 μg/L, between about 430 μg/L and about 440 μg/L, between about 430 μg/L and about 450 μg/L, between about 440 μg/L and about 450 μg/L), more than 450 μg/L (e.g., between about 450 μg/L and about 460 μg/L, between about 450 μg/L and about 470 μg/L, between about 450 μg/L and about 480 μg/L, between about 450 μg/L and about 490 μg/L, between about 450 μg/L and about 500 μg/L, between about 460 μg/L and about 470 μg/L, between about 460 μg/L and about 480 μg/L, between about 460 μg/L and about 490 μg/L, between about 460 μg/L and about 500 μg/L, between about 470 μg/L and about 480 μg/L, between about 470 μg/L and about 490 μg/L, between about 470 μg/L and about 500 μg/L, between about 480 μg/L and about 490 μg/L, between about 480 μg/L and about 500 μg/L, between about 490 μg/L and about 500 μg/L), more than 500 μg/L (e.g., between about 500 μg/L and about 510 μg/L, between about 500 μg/L and about 520 μg/L, between about 500 μg/L and about 530 μg/L, between about 500 μg/L and about 540 μg/L, between about 500 μg/L and about 550 μg/L, between about 510 μg/L and about 520 μg/L, between about 510 μg/L and about 530 μg/L, between about 510 μg/L and about 540 μg/L, between about 510 μg/L and about 550 μg/L, between about 520 μg/L and about 530 μg/L, between about 520 μg/L and about 540 μg/L, between about 520 μg/L and about 550 μg/L, between about 530 μg/L and about 540 μg/L, between about 530 μg/L and about 550 μg/L, between about 540 μg/L and about 550 μg/L), more than 550 μg/L (e.g., between about 550 μg/L and about 560 μg/L, between about 550 μg/L and about 570 μg/L, between about 550 μg/L and about 580 μg/L, between about 550 μg/L and about 590 μg/L, between about 550 μg/L and about 600 μg/L, between about 560 μg/L and about 570 μg/L, between about 560 μg/L and about 580 μg/L, between about 560 μg/L and about 590 μg/L, between about 560 μg/L and about 600 μg/L, between about 570 μg/L and about 580 μg/L, between about 570 μg/L and about 590 μg/L, between about 570 μg/L and about 600 μg/L, between about 580 μg/L and about 590 μg/L, between about 580 μg/L and about 600 μg/L, between about 590 μg/L and about 600 μg/L), more than 600 μg/L (e.g., between about 600 μg/L and about 610 μg/L, between about 600 μg/L and about 620 μg/L, between about 600 μg/L and about 630 μg/L, between about 600 μg/L and about 640 μg/L, between about 600 μg/L and about 650 μg/L, between about 610 μg/L and about 620 μg/L, between about 610 μg/L and about 630 μg/L, between about 610 μg/L and about 640 μg/L, between about 610 μg/L and about 650 μg/L, between about 620 μg/L and about 630 μg/L, between about 620 μg/L and about 640 μg/L, between about 620 μg/L and about 650 μg/L, between about 630 μg/L and about 640 μg/L, between about 630 μg/L and about 650 μg/L, between about 640 μg/L and about 650 μg/L), more than 650 μg/L (e.g., between about 350 μg/L and about 360 μg/L, between about 350 μg/L and about 370 μg/L, between about 350 μg/L and about 380 μg/L, between about 350 μg/L and about 390 μg/L, between about 350 μg/L and about 400 μg/L, between about 360 μg/L and about 370 μg/L, between about 360 μg/L and about 380 μg/L, between about 360 μg/L and about 390 μg/L, between about 360 μg/L and about 400 μg/L, between about 370 μg/L and about 380 μg/L, between about 370 μg/L and about 390 μg/L, between about 370 μg/L and about 400 μg/L, between about 380 μg/L and about 390 μg/L, between about 380 μg/L and about 400 μg/L, between about 390 μg/L and about 400 μg/L), more than 700 μg/L (e.g., between about 700 μg/L and about 710 μg/L, between about 700 μg/L and about 720 μg/L, between about 700 μg/L and about 730 μg/L, between about 700 μg/L and about 740 μg/L, between about 700 μg/L and about 750 μg/L, between about 710 μg/L and about 720 μg/L, between about 710 μg/L and about 730 μg/L, between about 710 μg/L and about 740 μg/L, between about 710 μg/L and about 750 μg/L, between about 720 μg/L and about 730 μg/L, between about 720 μg/L and about 740 μg/L, between about 720 μg/L and about 750 μg/L, between about 730 μg/L and about 740 μg/L, between about 730 μg/L and about 750 μg/L, between about 740 μg/L and about 750 μg/L), more than 750 μg/L (e.g., between about 750 μg/L and about 760 μg/L, between about 750 μg/L and about 770 μg/L, between about 750 μg/L and about 780 μg/L, between about 750 μg/L and about 790 μg/L, between about 750 μg/L and about 800 μg/L, between about 760 μg/L and about 770 μg/L, between about 760 μg/L and about 780 μg/L, between about 760 μg/L and about 790 μg/L, between about 760 μg/L and about 800 μg/L, between about 770 μg/L and about 780 μg/L, between about 770 μg/L and about 790 μg/L, between about 770 μg/L and about 800 μg/L, between about 780 μg/L and about 790 μg/L, between about 780 μg/L and about 800 μg/L, between about 790 μg/L and about 800 μg/L), more than 800 μg/L (e.g., between about 800 μg/L and about 810 μg/L, between about 800 μg/L and about 820 μg/L, between about 800 μg/L and about 830 μg/L, between about 800 μg/L and about 840 μg/L, between about 800 μg/L and about 850 μg/L, between about 810 μg/L and about 820 μg/L, between about 810 μg/L and about 830 μg/L, between about 810 μg/L and about 840 μg/L, between about 810 μg/L and about 850 μg/L, between about 820 μg/L and about 830 μg/L, between about 820 μg/L and about 840 μg/L, between about 820 μg/L and about 850 μg/L, between about 830 μg/L and about 840 μg/L, between about 830 μg/L and about 850 μg/L, between about 840 μg/L and about 850 μg/L), more than 850 μg/L (e.g., between about 850 μg/L and about 860 μg/L, between about 850 μg/L and about 870 μg/L, between about 850 μg/L and about 880 μg/L, between about 850 μg/L and about 890 μg/L, between about 850 μg/L and about 900 μg/L, between about 860 μg/L and about 870 μg/L, between about 860 μg/L and about 880 μg/L, between about 860 μg/L and about 890 μg/L, between about 860 μg/L and about 800 μg/L, between about 870 μg/L and about 880 μg/L, between about 870 μg/L and about 890 μg/L, between about 870 μg/L and about 900 μg/L, between about 880 μg/L and about 890 μg/L, between about 880 μg/L and about 900 μg/L, between about 890 μg/L and about 900 μg/L), more than 900 μg/L (e.g., between about 900 μg/L and about 910 μg/L, between about 900 μg/L and about 920 μg/L, between about 900 μg/L and about 930 μg/L, between about 900 μg/L and about 940 μg/L, between about 900 μg/L and about 950 μg/L, between about 910 μg/L and about 920 μg/L, between about 910 μg/L and about 930 μg/L, between about 910 μg/L and about 940 μg/L, between about 910 μg/L and about 950 μg/L, between about 920 μg/L and about 930 μg/L, between about 920 μg/L and about 940 μg/L, between about 920 μg/L and about 950 μg/L, between about 930 μg/L and about 940 μg/L, between about 930 μg/L and about 950 μg/L, between about 940 μg/L and about 950 μg/L), more than 950 μg/L (e.g., between about 950 μg/L and about 960 μg/L, between about 950 μg/L and about 970 μg/L, between about 950 μg/L and about 380 μg/L, between about 950 μg/L and about 990 μg/L, between about 950 μg/L and about 1000 μg/L, between about 960 μg/L and about 970 μg/L, between about 960 μg/L and about 980 μg/L, between about 960 μg/L and about 990 μg/L, between about 960 μg/L and about 1000 μg/L, between about 970 μg/L and about 980 μg/L, between about 970 μg/L and about 990 μg/L, between about 970 μg/L and about 1000 μg/L, between about 980 μg/L and about 990 μg/L, between about 980 μg/L and about 1000 μg/L, between about 990 μg/L and about 1000 μg/L), or more than 1000 μg/L (e.g., between about 1000 μg/L and about 1500 μg/L, between about 1000 μg/L and about 2000 μg/L, between about 1000 μg/L and about 2500 μg/L, between about 1000 μg/L and about 3000 μg/L, between about 1000 μg/L and about 4000 μg/L, between about 1000 μg/L and about 5000 μg/L, between about 2000 μg/L and about 3000 μg/L, between about 2000 μg/L and about 4000 μg/L, between about 2000 μg/L and about 5000 μg/L, between about 3000 μg/L and about 4000 μg/L, between about 4000 μg/L and about 5000 μg/L). In some embodiments, the subject has a serum ferritin level between about 100 μg/L and about 1000 μg/L, between about 100 μg/L and about 500 μg/L, between about 100 μg/L and about 500 μg/L, between about 200 μg/L and about 1000 μg/L, between about 200 μg/L and about 500 μg/L, between about 300 μg/L and about 1000 μg/L, between about 300 μg/L and about 500 μg/L, between about 400 μg/L and about 1000 μg/L, between about 400 μg/L and about 500 μg/L. It should be appreciated, however, that other suitable markers (e.g., TSAT %, serum iron levels, total iron binding capacity (TIBC), hemoglobin levels, hepatic iron content, Reticulocytes Hemoglobin Content, hepcidin levels, IL-6 levels, creatinine levels, etc.) may be evaluated to determine if the subject is suitable for method of treatment described herein.

In some embodiments, the ACD is characterized based on Reticulocytes Hemoglobin Content (RET-He or CHr). Reticulocyte hemoglobin content measures the amount of hemoglobin in reticulocytes. Normal range of CHr is about 28 to 36 pg/cell. In some embodiments, the subject has a CHr lower than the normal range. In some embodiments, the subject has a CHr less than 36 pg/cell, less than 35 pg/cell, less than 34 pg/cell, less than 33 pg/cell. less than 32 pg/cell, 31 pg/cell, 30 pg/cell, 29 pg/cell, less than 28 pg/cell, less than 27 pg/cell, less than 26 pg/cell, less than 25 pg/cell, less than 24 pg/cell, less than 23 pg/cell, less than 21 pg/cell, less than 20 pg/cell, less than 19 pg/cell, less than 18 pg/cell, less than 17 pg/cell, less than 16 pg/cell, less than 15 pg/cell, less than 14 pg/cell, less than 13 pg/cell, less than 12 pg/cell, less than 11 pg/cell, less than 10 pg/cell, less than 9 pg/cell, less than 8 pg/cell, less than 7 pg/cell, less than 6 pg/cell, less than 5 pg/cell, less than 4 pg/cell, less than 3 pg/cell, less than 2 pg/cell, or less than 1 pg/cell. In some embodiments, the subject has a CHr lower than the normal range. In some embodiments, the subject has a CHr between about 1 pg/cell to about 36 pg/cell, between about 1 pg/cell to about 32 pg/cell, between about 1 pg/cell to about 30 pg/cell, between about 1 pg/cell to about 28 pg/cell, between about 1 pg/cell to about 25 pg/cell, between about 1 pg/cell to about 20 pg/cell, between about 1 pg/cell to about 15 pg/cell, between about 1 pg/cell to about 12 pg/cell, between about 1 pg/cell to about 10 pg/cell, between about 1 pg/cell to about 8 pg/cell, between about 1 pg/cell to about 6 pg/cell, between about 1 pg/cell to about 4 pg/cell, between about 5 pg/cell to about 36 pg/cell, between about 5 pg/cell to about 32 pg/cell, between about 5 pg/cell to about 30 pg/cell, between about 5 pg/cell to about 28 pg/cell, between about 5 pg/cell to about 25 pg/cell, between about 5 pg/cell to about 20 pg/cell, between about 5 pg/cell to about 15 pg/cell, between about 5 pg/cell to about 12 pg/cell, between about 5 pg/cell to about 10 pg/cell, between about 5 pg/cell to about 8 pg/cell, between about 5 pg/cell to about 6 pg/cell, between about 10 pg/cell to about 36 pg/cell, between about 10 pg/cell to about 32 pg/cell, between about 10 pg/cell to about 30 pg/cell, between about 1 pg/cell to about 28 pg/cell, between about 10 pg/cell to about 25 pg/cell, between about 10 pg/cell to about 20 pg/cell, between about 10 pg/cell to about 15 pg/cell, between about 10 pg/cell to about 12 pg/cell, between about 15 pg/cell to about 36 pg/cell, between about 15 pg/cell to about 32 pg/cell, between about 15 pg/cell to about 30 pg/cell, between about 15 pg/cell to about 28 pg/cell, between about 15 pg/cell to about 25 pg/cell, between about 15 pg/cell to about 20 pg/cell, between about 20 pg/cell to about 36 pg/cell, between about 20 pg/cell to about 32 pg/cell, between about 20 pg/cell to about 30 pg/cell, between about 20 pg/cell to about 28 pg/cell, between about 20 pg/cell to about 25 pg/cell, between about 25 pg/cell to about 36 pg/cell, between about 25 pg/cell to about 32 pg/cell, between about 25 pg/cell to about 30 pg/cell, between about 25 pg/cell to about 28 pg/cell, between about 30 pg/cell to about 36 pg/cell, between about 30 pg/cell to about 32 pg/cell. It should be appreciated, however, that other suitable markers (e.g., TSAT %, serum iron levels, total iron binding capacity (TIBC), ferritin levels, hemoglobin levels, hepatic iron content, hepcidin levels, IL-6 levels, creatinine levels, etc.) may be evaluated to determine if the subject is suitable for method of treatment described herein.

In some embodiments, the ACD is characterized by hepatic iron levels. In some embodiments, a normal range of hepatic iron level is 200-2,400 μg/g dry weight in males and 400-1,600 μg/g dry weight in female. In some embodiments, the subject has a higher than normal of hepatic iron level. In some embodiments, the patient has hepatic iron level more than 200 μg/g dry weight (e.g., between about 200 μg/g to 250 μg/g dry weight, between about 200 μg/g to 250 μg/g dry weight, between about 200 μg/g to 300 μg/g dry weight, between about 220 μg/g to 250 μg/g dry weight, between about 220 μg/g to 300 μg/g dry weight, between about 250 μg/g to 300 μg/g dry weight, between about 260 μg/g to 300 μg/g dry weight, or between about 280 μg/g to 300 μg/g dry weight), more than 300 μg/g dry weight (e.g., between about 300 μg/g to 320 μg/g dry weight, between about 300 μg/g to 350 μg/g dry weight, between about 300 μg/g to 400 μg/g dry weight, between about 320 μg/g to 350 μg/g dry weight, between about 320 μg/g to 400 μg/g dry weight, between about 350 μg/g to 400 μg/g dry weight, between about 360 μg/g to 400 μg/g dry weight, or between about 380 μg/g to 400 μg/g dry weight), more than 400 μg/g dry weight (e.g., between about 400 μg/g to 420 μg/g dry weight, between about 400 μg/g to 450 μg/g dry weight, between about 400 μg/g to 500 μg/g dry weight, between about 420 μg/g to 450 μg/g dry weight, between about 420 μg/g to 500 μg/g dry weight, between about 450 μg/g to 500 μg/g dry weight, between about 460 μg/g to 500 μg/g dry weight, or between about 480 μg/g to 500 μg/g dry weight), more than 500 μg/g dry weight (e.g., between about 500 μg/g to 520 μg/g dry weight, between about 500 μg/g to 550 μg/g dry weight, between about 500 μg/g to 600 μg/g dry weight, between about 520 μg/g to 550 μg/g dry weight, between about 520 μg/g to 600 μg/g dry weight, between about 550 μg/g to 600 μg/g dry weight, between about 560 μg/g to 600 μg/g dry weight, or between about 580 μg/g to 600 μg/g dry weight), more than 600 μg/g dry weight (e.g., between about 600 μg/g to 620 μg/g dry weight, between about 600 μg/g to 650 μg/g dry weight, between about 600 μg/g to 700 μg/g dry weight, between about 620 μg/g to 650 μg/g dry weight, between about 620 μg/g to 700 μg/g dry weight, between about 650 μg/g to 700 μg/g dry weight, between about 660 μg/g to 700 μg/g dry weight, or between about 680 μg/g to 700 μg/g dry weight), more than 700 μg/g dry weight (e.g., between about 700 μg/g to 720 μg/g dry weight, between about 700 μg/g to 750 μg/g dry weight, between about 700 μg/g to 800 μg/g dry weight, between about 720 μg/g to 750 μg/g dry weight, between about 720 μg/g to 800 μg/g dry weight, between about 750 μg/g to 800 μg/g dry weight, between about 760 μg/g to 800 μg/g dry weight, or between about 780 μg/g to 800 μg/g dry weight), more than 800 μg/g dry weight (e.g., between about 800 μg/g to 820 μg/g dry weight, between about 800 μg/g to 850 μg/g dry weight, between about 800 μg/g to 900 μg/g dry weight, between about 820 μg/g to 850 μg/g dry weight, between about 820 μg/g to 900 μg/g dry weight, between about 850 μg/g to 900 μg/g dry weight, between about 860 μg/g to 900 μg/g dry weight, or between about 880 μg/g to 900 μg/g dry weight), more than 900 μg/g dry weight (e.g., between about 900 μg/g to 920 μg/g dry weight, between about 900 μg/g to 950 μg/g dry weight, between about 900 μg/g to 1000 μg/g dry weight, between about 920 μg/g to 950 μg/g dry weight, between about 920 μg/g to 1000 μg/g dry weight, between about 950 μg/g to 1000 μg/g dry weight, between about 960 μg/g to 1000 μg/g dry weight, or between about 980 μg/g to 1000 μg/g dry weight), more than 1000 μg/g dry weight (e.g., between about 1000 μg/g to 1200 μg/g dry weight, between about 1000 μg/g to 1500 μg/g dry weight, or between about 1200 μg/g to 1500 μg/g dry weight), more than 1500 μg/g dry weight (e.g., between about 1500 μg/g to 1800 μg/g dry weight, between about 1500 μg/g to 2000 μg/g dry weight, or between about 1800 μg/g to 2000 μg/g dry weight), more than 2000 μg/g dry weight (e.g., between about 2000 μg/g to 2200 μg/g dry weight, between about 2000 μg/g to 2500 μg/g dry weight, or between about 2200 μg/g to 2500 μg/g dry weight), more than 2500 μg/g dry weight (e.g., between about 2500 μg/g to 2800 μg/g dry weight, between about 2500 μg/g to 3000 μg/g dry weight, or between about 2800 μg/g to 3000 μg/g dry weight), more than 3000 μg/g dry weight (e.g., between about 3000 μg/g to 3200 μg/g dry weight, between about 3000 μg/g to 3500 μg/g dry weight, or between about 3200 μg/g to 3500 μg/g dry weight), more than 3500 μg/g dry weight (e.g., between about 3500 μg/g to 3800 μg/g dry weight, between about 3500 μg/g to 4000 μg/g dry weight, or between about 3800 μg/g to 4000 μg/g dry weight), more than 4000 μg/g dry weight (e.g., between about 4000 μg/g to 4200 μg/g dry weight, between about 4000 μg/g to 4500 μg/g dry weight, or between about 4200 μg/g to 4500 μg/g dry weight), more than 4500 μg/g dry weight (e.g., between about 4500 μg/g to 4800 μg/g dry weight, between about 4500 μg/g to 5000 μg/g dry weight, or between about 4800 μg/g to 5000 μg/g dry weight), more than 5000 μg/g dry weight (e.g., between about 5000 μg/g to 5200 μg/g dry weight, between about 5000 μg/g to 5500 μg/g dry weight, or between about 5200 μg/g to 5500 μg/g dry weight), more than 5500 μg/g dry weight (e.g., between about 5500 μg/g to 5800 μg/g dry weight, between about 5500 μg/g to 6000 μg/g dry weight, or between about 5800 μg/g to 6000 μg/g dry weight), more than 6000 μg/g dry weight (e.g., between about 6000 μg/g to 6200 μg/g dry weight, between about 6000 μg/g to 6500 μg/g dry weight, or between about 6200 μg/g to 6500 μg/g dry weight), more than 6500 μg/g dry weight (e.g., between about 6500 μg/g to 6800 μg/g dry weight, between about 6500 μg/g to 7000 μg/g dry weight, or between about 6800 μg/g to 7000 μg/g dry weight), more than 7000 μg/g dry weight (e.g., between about 7000 μg/g to 7200 μg/g dry weight, between about 7000 μg/g to 7500 μg/g dry weight, or between about 7200 μg/g to 7500 μg/g dry weight), more than 7500 μg/g dry weight (e.g., between about 7500 μg/g to 7800 μg/g dry weight, between about 7500 μg/g to 8000 μg/g dry weight, or between about 7800 μg/g to 8000 μg/g dry weight), more than 8000 μg/g dry weight (e.g., between about 8000 μg/g to 8200 μg/g dry weight, between about 8000 μg/g to 8500 μg/g dry weight, or between about 8200 μg/g to 8500 μg/g dry weight), more than 8500 μg/g dry weight (e.g., between about 8500 μg/g to 8800 μg/g dry weight, between about 8500 μg/g to 9000 μg/g dry weight, or between about 8800 μg/g to 9000 μg/g dry weight), more than 9000 μg/g dry weight (e.g., between about 9000 μg/g to 9200 μg/g dry weight, between about 9000 μg/g to 9500 μg/g dry weight, or between about 9200 μg/g to 9500 μg/g dry weight), more than 9500 μg/g dry weight (e.g., between about 9500 μg/g to 9800 μg/g dry weight, between about 9000 μg/g to 10000 μg/g dry weight, or between about 9800 μg/g to 10000 μg/g dry weight), or more than 10000 μg/g dry weight (e.g., between about 10000 μg/g to 15000 μg/g dry weight, between about 10000 μg/g to 20000 μg/g dry weight, or between about 10000 μg/g to 15000 μg/g dry weight). In some embodiments, the patient has hepatic iron level between about 200 μg/g and about 500 μg/g dry weight, between about 200 μg/g and about 1000 μg/g dry weight, between about 200 μg/g and about 2000 μg/g dry weight, between about 200 μg/g and about 5000 μg/g dry weight, between about 200 μg/g and about 8000 μg/g dry weight, between about 200 μg/g and about 10000 μg/g dry weight, between about 500 μg/g and about 1000 μg/g dry weight, between about 500 μg/g and about 5000 μg/g dry weight, between about 500 μg/g and about 10000 μg/g dry weight, between about 1000 μg/g and about 2000 μg/g dry weight, between about 1000 μg/g and about 5000 μg/g dry weight, between about 1000 μg/g and about 10000 μg/g dry weight, between about 5000 μg/g and about 8000 μg/g dry weight, or between about 5000 μg/g and about 10000 μg/g dry weight. It should be appreciated, however, that other suitable markers (e.g., TSAT %, serum iron levels, total iron binding capacity (TIBC), ferritin levels, hemoglobin levels, Reticulocytes Hemoglobin Content, hepcidin levels, IL-6 levels, creatinine levels, etc.) may be evaluated to determine if the subject is suitable for method of treatment described herein.

In some embodiments, the ACD is also characterized by low serum iron levels. In some embodiments, a normal range of serum iron level is 50-150 μg/dL in males and 35-145 μg/dL dry weight in female. In some embodiments, the subject has a lower than normal serum iron level. In some embodiments, the subject has a serum iron level of less than 150 μg/dL, less than 140 μg/dL, less than 130 μg/dL, less than 120 μg/dL, less than 110 μg/dL, less than 100 μg/dL, less than 90 μg/dL, less than 80 μg/dL, less than 70 μg/dL, less than 60 μg/dL, less than 50 μg/dL, less than 45 μg/dL, less than 40 μg/dL, less than 35 μg/dL, less than 30 μg/dL, less than 25 μg/dL, less than 20 μg/dL, less than 15 μg/dL, less than 10 μg/dL, or less than 5 μg/dL. In some embodiments, the subject has a serum iron level of between about 1 μg/dL and about 150 μg/dL, between about 5 μg/dL and about 150 μg/dL, between about 10 μg/dL and about 150 μg/dL, between about 20 μg/dL and about 150 μg/dL, between about 50 μg/dL and about 150 μg/dL, between about 80 μg/dL and about 150 μg/dL, between about 100 μg/dL and about 150 μg/dL, between about 120 μg/dL and about 150 μg/dL, between about 1 μg/dL and about 120 μg/dL, between about 5 μg/dL and about 120 μg/dL, between about 10 μg/dL and about 120 μg/dL, between about 20 μg/dL and about 120 μg/dL, between about 50 μg/dL and about 120 μg/dL, between about 80 μg/dL and about 120 μg/dL, between about 120 μg/dL and about 120 μg/dL, between about 1 μg/dL and about 100 μg/dL, between about 5 μg/dL and about 100 μg/dL, between about 10 μg/dL and about 100 μg/dL, between about 20 μg/dL and about 100 μg/dL, between about 50 μg/dL and about 100 μg/dL, between about 80 μg/dL and about 100 μg/dL, between about 1 μg/dL and about 80 μg/dL, between about 5 μg/dL and about 80 μg/dL, between about 10 μg/dL and about 80 μg/dL, between about 20 μg/dL and about 80 μg/dL, between about 50 μg/dL and about 80 μg/dL, between about 1 μg/dL and about 50 μg/dL, between about 5 μg/dL and about 50 μg/dL, between about 10 μg/dL and about 50 μg/dL, between about 20 μg/dL and about 50 μg/dL, between about 25 μg/dL and about 50 μg/dL, between about 30 μg/dL and about 50 μg/dL, between about 1 μg/dL and about 25 μg/dL, between about 5 μg/dL and about 25 μg/dL, between about 10 μg/dL and about 25 μg/dL, between about 10 μg/dL and about 20 μg/dL, between about 10 μg/dL and about 15 μg/dL, between about 1 μg/dL and about 20 μg/dL, between about 5 μg/dL and about 18 μg/dL, between about 10 μg/dL and about 16 μg/dL, between about 1 μg/dL and about 15 μg/dL, between about 5 μg/dL and about 12 μg/dL, between about 10 μg/dL and about 12 μg/dL, between about 1 μg/dL and about 10 μg/dL, between about 1 μg/dL and about 8 μg/dL, between about 1 μg/dL and about 5 μg/dL, or between about 1 μg/dL and about 3 μg/dL. It should be appreciated, however, that other suitable markers (e.g., TSAT %, total iron binding capacity (TIBC), ferritin levels, hemoglobin levels, hepatic iron content, Reticulocytes Hemoglobin Content, hepcidin levels, IL-6 levels, creatinine levels, etc.) may be evaluated to determine if the subject is suitable for method of treatment described herein.

In some embodiments, the ACD is characterized by low Total Iron Binding Capacity (TIBC). In some embodiments, normal range of TIBC is 250-400 μg/dL. In some embodiments, the subject has a lower than normal TIBC. In some embodiments, the subject has a TIBC of less than 400 μg/dL, less than 350 μg/dL, less than 300 μg/dL, less than 250 μg/dL, less than 200 μg/dL, less than 150 μg/dL, less than 100 μg/dL, less than 90 μg/dL, less than 80 μg/dL, less than 70 μg/dL, less than 60 μg/dL, less than 50 μg/dL, less than 40 μg/dL, less than 30 μg/dL less than 20 μg/dL, or less than 10 μg/dL. In some embodiments, the subject has a TIBC of between about 1 μg/dL and about 400 μg/dL, between about 1 μg/dL and about 300 μg/dL, between about 1 μg/dL and about 200 μg/dL, between about 1 μg/dL and about 100 μg/dL, between about 1 μg/dL and about 50 μg/dL, between about 1 μg/dL and about 25 μg/dL, between about 1 μg/dL and about 10 μg/dL, between about 1 μg/dL and about 5 μg/dL, between about 5 μg/dL and about 400 μg/dL, between about 5 μg/dL and about 300 μg/dL, between about 5 μg/dL and about 200 μg/dL, between about 5 μg/dL and about 100 μg/dL, between about 5 μg/dL and about 50 μg/dL, between about 5 μg/dL and about 25 μg/dL, between about 5 μg/dL and about 10 μg/dL, between about 10 μg/dL and about 400 μg/dL, between about 10 μg/dL and about 300 μg/dL, between about 10 μg/dL and about 200 μg/dL, between about 10 μg/dL and about 100 μg/dL, between about 10 μg/dL and about 50 μg/dL, between about 10 μg/dL and about 25 μg/dL, between about 25 μg/dL and about 400 μg/dL, between about 25 μg/dL and about 300 μg/dL, between about 25 μg/dL and about 200 μg/dL, between about 25 μg/dL and about 100 μg/dL, between about 25 μg/dL and about 50 μg/dL, between about 50 μg/dL and about 400 μg/dL, between about 50 μg/dL and about 300 μg/dL, between about 50 μg/dL and about 200 μg/dL, between about 50 μg/dL and about 100 μg/dL, between about 100 μg/dL and about 400 μg/dL, between about 100 μg/dL and about 300 μg/dL, between about 100 μg/dL and about 200 μg/dL, between about 100 μg/dL and about 150 μg/dL, between about 100 μg/dL and about 250 μg/dL, between about 100 μg/dL and about 350 μg/dL, between about 200 μg/dL and about 250 μg/dL, between about 200 μg/dL and about 300 μg/dL, between about 200 μg/dL and about 350 μg/dL, between about 200 μg/dL and about 400 μg/dL, between about 300 μg/dL and about 350 μg/dL, or between about 350 μg/dL and about 450 μg/dL. It should be appreciated, however, that other suitable markers (e.g., TSAT %, serum iron levels, ferritin levels, hemoglobin levels, hepatic iron content, Reticulocytes Hemoglobin Content, hepcidin levels, IL-6 levels, creatinine levels, etc.) may be evaluated to determine if the subject is suitable for method of treatment described herein.

In some embodiments, the ACD is characterized based on a transferrin saturation level (TSAT %). In some embodiments, a normal range of TSAT is about 20%-50%. In some embodiments, transferrin saturations of less than 20% indicate iron deficiency, while in some embodiments, transferrin saturations of more than 50% suggest iron overload. In some embodiments, the subject has a TSAT % less than 100%, less than 90%, less than 80%, less than 70%, less than 60%, less than 50%, less than 40%, less than 30%, less than 20%, or less than 10%. In some embodiments, the subject has a TSAT % between 5%-10%, between 5%-20%, between 5%-30%, between 5%-40%, between 5%-50%, between 5%-60%, between 5%-70%, between 8%-10%, between 8%-20%, between 8%-30%, between 8%-40%, between 8%-50%, between 8%-60%, between 8%-70%, between 10%-15%, between 10%-20%, between 10%-30%, between 10%-40%, between 10%-50%, between 10%-60%, between 10%-70%, between 15%-20%, between 15%-25%, between 15%-30%, between 15%-40%, between 15%-50%, between 15%-60%, between 15%-70%, between 20%-25%, between 20%-30%, between 20%-33%, between 20%-40%, between 20%-50%, between 20%-60%, between 20%-70%, between 25%-30%, between 25%-35%, between 25%-40%, between 25%-50%, between 25%-60%, between 25%-70%, between 30%-40%, between 30%-50%, between 30%-55%, between 30%-60%, between 30%-70%, between 35%-40%, between 35%-50%, between 35%-55%, between 35%-60%, between 35%-70%, between 40%-50%, between 40%-55%, between 40%-60%, between 40%-70%, between 50%-55%, between 50%-60%, or between 50%-70%. In some cases, if TSAT % of the subject is at or above 70%, at or above 75%, at or above 80%, at or above 85%, at or above 90%, or at or above 95%, an ongoing treatment with an hepcidin antagonist may be stopped or temporarily stopped, e.g., to prevent iron overload. In other embodiments, administration of an hepcidin antagonist may be performed when a TSAT % of a subject is at or below 95%, at or below 90%, at or below 80%, at or below 70%, at or below 65%, at or below 60%, at or below 55%, at or below 50%, at or below 45%, at or below 40%, at or below 35%, or at or below 30%. Thus, in some embodiments, TSAT % of a subject can be monitored, e.g., continuously or periodically, while a patient is receiving a treatment or under care of a treating physician, e.g., for anemia, to prevent iron overload or otherwise to assess whether further treatments are appropriate. It should be appreciated, however, that other suitable markers (e.g., serum iron levels, total iron binding capacity (TIBC), ferritin levels, hemoglobin levels, hepatic iron content, Reticulocytes Hemoglobin Content, hepcidin levels, IL-6 levels, creatinine levels, etc.) may be evaluated to determine if the subject is suitable for method of treatment described herein.

In some embodiments, the ACD is also characterized by high serum hepcidin levels. In some embodiments, a normal range of hepcidin is 1-55 ng/ml. In some embodiments, the subject has a higher than normal serum hepcidin levels. In some embodiments, the subject has a serum hepcidin level of more than 55 ng/ml, more than 55 ng/ml, more than 60 ng/ml, more than 65 ng/ml, more than 70 ng/ml, more than 75 ng/ml, more than 80 ng/ml, more than 85 ng/ml, more than 90 ng/ml, more than 95 ng/ml, more than 100 ng/ml, more than 150 ng/ml, more than 200 ng/ml, more than 250 ng/ml, more than 300 ng/ml, more than 350 ng/ml, more than 400 ng/ml, more than 450 ng/ml, or more than 500 ng/ml. In some embodiments, the subject has a serum hepcidin level of between about 55 ng/ml and about 1000 ng/ml, between about 55 ng/ml and about 800 ng/ml, between about 55 ng/ml and about 600 ng/ml, between about 55 ng/ml and about 500 ng/ml, between about 55 ng/ml and about 400 ng/ml, between about 55 ng/ml and about 300 ng/ml, between about 55 ng/ml and about 250 ng/ml, between about 55 ng/ml and about 300 ng/ml, between about 55 ng/ml and about 200 ng/ml, between about 55 ng/ml and about 250 ng/ml, between about 55 ng/ml and about 200 ng/ml, between about 55 ng/ml and about 150 ng/ml, between about 55 ng/ml and about 100 ng/ml, between about 55 ng/ml and about 80 ng/ml, between about 55 ng/ml and about 75 ng/ml, between about 100 ng/ml and about 1000 ng/ml, between about 100 ng/ml and about 800 ng/ml, between about 100 ng/ml and about 600 ng/ml, between about 100 ng/ml and about 500 ng/ml, between about 100 ng/ml and about 400 ng/ml, between about 100 ng/ml and about 300 ng/ml, between about 100 ng/ml and about 250 ng/ml, between about 100 ng/ml and about 300 ng/ml, between about 100 ng/ml and about 200 ng/ml, between about 100 ng/ml and about 250 ng/ml, between about 100 ng/ml and about 200 ng/ml, between about 100 ng/ml and about 150 ng/ml, between about 100 ng/ml and about 125 ng/ml, between about 200 ng/ml and about 1000 ng/ml, between about 200 ng/ml and about 800 ng/ml, between about 200 ng/ml and about 600 ng/ml, between about 200 ng/ml and about 500 ng/ml, between about 200 ng/ml and about 400 ng/ml, between about 200 ng/ml and about 300 ng/ml, between about 200 ng/ml and about 250 ng/ml, between about 300 ng/ml and about 1000 ng/ml, between about 300 ng/ml and about 800 ng/ml, between about 300 ng/ml and about 600 ng/ml, between about 300 ng/ml and about 500 ng/ml, between about 300 ng/ml and about 400 ng/ml, between about 300 ng/ml and about 350 ng/ml, between about 400 ng/ml and about 1000 ng/ml, between about 400 ng/ml and about 800 ng/ml, between about 400 ng/ml and about 600 ng/ml, between about 400 ng/ml and about 500 ng/ml, between about 400 ng/ml and about 450 ng/ml, between about 800 ng/ml and about 1000 ng/ml, between about 800 ng/ml and about 900 ng/ml, between about 800 ng/ml and about 850 ng/ml, between about 900 ng/ml and about 1000 ng/ml, or between about 900 ng/ml and about 950 ng/ml. It should be appreciated, however, that other suitable markers (e.g., serum iron levels, total iron binding capacity (TIBC), ferritin levels, hemoglobin levels, hepatic iron content, Reticulocytes Hemoglobin Content, IL-6 levels, creatinine levels, etc.) may be evaluated to determine if the subject is suitable for method of treatment described herein.

In some embodiments, the ACD is characterized by high serum creatinine levels. In some embodiments, a normal range of serum creatinine is about 0.84 to 1.21 mg/dL. In some embodiments, the subject has a higher than normal serum creatinine levels. In some embodiments, the subject has a serum creatinine level of more than 1 mg/dL, more than 1.5 mg/dL, more than 2 mg/dL, more than 2.5 mg/dL, more than 3 mg/dL, more than 3.5 mg/dL, more than 4 mg/dL, more than 4.5 mg/dL, more than 5 mg/dL, more than 5.5 mg/dL, more than 6 mg/dL, more than 6.5 mg/dL, more than 7 mg/dL, more than 7.5 mg/dL, more than 8 mg/dL, more than 8.5 mg/dL, more than 9 mg/dL, more than 9.5 mg/dL, more than 10 mg/dL, more than 15 mg/dL, more than 20 mg/dL, more than 30 mg/dL, more than 40 mg/dL, more than 50 mg/dL, more than 60 mg/dL, more than 70 mg/dL, more than 80 mg/dL, more than 90 mg/dL, or more than 100 mg/dL. In some embodiments, the subject has a serum creatinine level of between about 1 mg/dl and about 200 mg/dL, 1 mg/dl and about 175 mg/dL, 1 mg/dl and about 150 mg/dL, 1 mg/dl and about 100 mg/dL, 1 mg/dl and about 50 mg/dL, 1 mg/dl and about 25 mg/dL, 1 mg/dl and about 10 mg/dL, 1 mg/dl and about 5 mg/dL, 1 mg/dl and about 2 mg/dL, between about 5 mg/dl and about 200 mg/dL, 5 mg/dl and about 175 mg/dL, 5 mg/dl and about 150 mg/dL, 5 mg/dl and about 100 mg/dL, 5 mg/dl and about 50 mg/dL, 5 mg/dl and about 25 mg/dL, 5 mg/dl and about 10 mg/dL, between about 10 mg/dl and about 200 mg/dL, 10 mg/dl and about 175 mg/dL, 10 mg/dl and about 150 mg/dL, 10 mg/dl and about 100 mg/dL, 10 mg/dl and about 50 mg/dL, 10 mg/dl and about 25 mg/dL, 10 mg/dl and about 20 mg/dL, 10 mg/dl and about 25 mg/dL, between about 20 mg/dl and about 200 mg/dL, 20 mg/dl and about 175 mg/dL, 20 mg/dl and about 150 mg/dL, 20 mg/dl and about 100 mg/dL, 20 mg/dl and about 50 mg/dL, 20 mg/dl and about 25 mg/dL, between about 50 mg/dl and about 200 mg/dL, 50 mg/dl and about 175 mg/dL, 50 mg/dl and about 150 mg/dL, 50 mg/dl and about 100 mg/dL, 50 mg/dl and about 75 mg/dL, between about 100 mg/dl and about 200 mg/dL, 100 mg/dl and about 175 mg/dL, 100 mg/dl and about 150 mg/dL, or 100 mg/dl and about 125 mg/dL. It should be appreciated, however, that other suitable markers (e.g., serum iron levels, total iron binding capacity (TIBC), ferritin levels, hemoglobin levels, hepatic iron content, Reticulocytes Hemoglobin Content, etc.) may be evaluated to determine if the subject is suitable for method of treatment described herein.

In some embodiments, the ACD is also characterized by high serum IL-6 levels. Normal range of IL-6 is equal or less than 1.8 pg/ml. In some embodiments, the subject has a higher than normal serum IL-6 levels. In some embodiments, the subject has a serum IL-6 level of more than 0.5 pg/ml, more than 0.6 pg/ml, more than 0.7 pg/ml, more than 0.8 pg/ml, more than 0.9 pg/ml, more than 1 pg/ml, more than 1.1 pg/ml, more than 1.2 pg/ml, more than 1.3 pg/ml, more than 1.4 pg/ml, more than 1.5 pg/ml, more than 1.6 pg/ml, more than 1.7 pg/ml, more than 1.8 pg/ml, more than 2 pg/ml, than 3 pg/ml, than 4 pg/ml, more than 5 pg/ml, more than 6 pg/ml, more than 7 pg/ml, more than 8 pg/ml, more than 9 pg/ml, more than 10 pg/ml, more than 20 pg/ml, more than 30 pg/ml, more than 40 pg/ml, more than 50 pg/ml, more than 60 pg/ml, more than 70 pg/ml, more than 80 pg/ml, more than 90 pg/ml, more than 100 pg/ml, more than 200 pg/ml, more than 300 pg/ml, more than 400 pg/ml, more than 500 pg/ml, more than 600 pg/ml, more than 700 pg/ml, more than 800 pg/ml, more than 900 pg/ml, or more than 1000 pg/ml. In some embodiments, the subject has a serum IL-6 level of between about 0.5 pg/ml and about 1500 pg/ml, between about 0.5 pg/ml and about 1000 pg/ml, between about 0.5 pg/ml and about 800 pg/ml, between about 0.5 pg/ml and about 750 pg/ml, between about 0.5 pg/ml and about 500 pg/ml, between about 0.5 pg/ml and about 250 pg/ml, between about 0.5 pg/ml and about 200 pg/ml, between about 0.5 pg/ml and about 150 pg/ml, between about 0.5 pg/ml and about 100 pg/ml, between about 0.5 pg/ml and about 50 pg/ml, between about 0.5 pg/ml and about 25 pg/ml, between about 0.5 pg/ml and about 10 pg/ml, between about 0.5 pg/ml and about 5 pg/ml, between about 0.5 pg/ml and about 2.5 pg/ml, between about 0.5 pg/ml and about 1 pg/ml, between about 1 pg/ml and about 1500 pg/ml, between about 1 pg/ml and about 1000 pg/ml, between about 1 pg/ml and about 800 pg/ml, between about 1 pg/ml and about 750 pg/ml, between about 1 pg/ml and about 500 pg/ml, between about 1 pg/ml and about 250 pg/ml, between about 1 pg/ml and about 200 pg/ml, between about 1 pg/ml and about 150 pg/ml, between about 1 pg/ml and about 100 pg/ml, between about 1 pg/ml and about 50 pg/ml, between about 1 pg/ml and about 25 pg/ml, between about 1 pg/ml and about 10 pg/ml, between about 1 pg/ml and about 5 pg/ml, between about 1 pg/ml and about 2.5 pg/ml, between about 1 pg/ml and about 2 pg/ml, between about 1.2 pg/ml and about 2 pg/ml, between about 1.5 pg/ml and about 2 pg/ml, between about 1.2 pg/ml and about 1.8 pg/ml, between about 2 pg/ml and about 1500 pg/ml, between about 2 pg/ml and about 1000 pg/ml, between about 2 pg/ml and about 800 pg/ml, between about 2 pg/ml and about 750 pg/ml, between about 2 pg/ml and about 500 pg/ml, between about 2 pg/ml and about 250 pg/ml, between about 2 pg/ml and about 200 pg/ml, between about 2 pg/ml and about 150 pg/ml, between about 2 pg/ml and about 100 pg/ml, between about 2 pg/ml and about 50 pg/ml, between about 2 pg/ml and about 25 pg/ml, between about 2 pg/ml and about 10 pg/ml, between about 2 pg/ml and about 5 pg/ml, between about 2 pg/ml and about 3 pg/ml, between about 2 pg/ml and about 4 pg/ml, between about 2 pg/ml and about 2.5 pg/ml, between about 5 pg/ml and about 1500 pg/ml, between about 5 pg/ml and about 1000 pg/ml, between about 5 pg/ml and about 800 pg/ml, between about 5 pg/ml and about 750 pg/ml, between about 5 pg/ml and about 500 pg/ml, between about 5 pg/ml and about 250 pg/ml, between about 5 pg/ml and about 200 pg/ml, between about 5 pg/ml and about 150 pg/ml, between about 5 pg/ml and about 100 pg/ml, between about 5 pg/ml and about 50 pg/ml, between about 5 pg/ml and about 25 pg/ml, between about 5 pg/ml and about 10 pg/ml, between about 5 pg/ml and about 7.5 pg/ml, between about 5 pg/ml and about 15 pg/ml, between about 5 pg/ml and about 20 pg/ml, between about 10 pg/ml and about 1500 pg/ml, between about 10 pg/ml and about 1000 pg/ml, between about 10 pg/ml and about 800 pg/ml, between about 10 pg/ml and about 750 pg/ml, between about 10 pg/ml and about 500 pg/ml, between about 10 pg/ml and about 250 pg/ml, between about 10 pg/ml and about 200 pg/ml, between about 10 pg/ml and about 150 pg/ml, between about 10 pg/ml and about 100 pg/ml, between about 10 pg/ml and about 50 pg/ml, between about 10 pg/ml and about 25 pg/ml, between about 10 pg/ml and about 15 pg/ml, between about 20 pg/ml and about 1500 pg/ml, between about 20 pg/ml and about 1000 pg/ml, between about 20 pg/ml and about 800 pg/ml, between about 20 pg/ml and about 750 pg/ml, between about 20 pg/ml and about 500 pg/ml, between about 20 pg/ml and about 250 pg/ml, between about 20 pg/ml and about 200 pg/ml, between about 20 pg/ml and about 150 pg/ml, between about 20 pg/ml and about 100 pg/ml, between about 20 pg/ml and about 50 pg/ml, between about 20 pg/ml and about 25 pg/ml, between about 30 pg/ml and about 1500 pg/ml, between about 30 pg/ml and about 1000 pg/ml, between about 30 pg/ml and about 800 pg/ml, between about 30 pg/ml and about 750 pg/ml, between about 30 pg/ml and about 500 pg/ml, between about 30 pg/ml and about 250 pg/ml, between about 30 pg/ml and about 200 pg/ml, between about 30 pg/ml and about 150 pg/ml, between about 30 pg/ml and about 100 pg/ml, between about 30 pg/ml and about 50 pg/ml, between about 30 pg/ml and about 45 pg/ml, between about 30 pg/ml and about 45 pg/ml, between about 50 pg/ml and about 1500 pg/ml, between about 50 pg/ml and about 1000 pg/ml, between about 50 pg/ml and about 800 pg/ml, between about 50 pg/ml and about 750 pg/ml, between about 50 pg/ml and about 500 pg/ml, between about 50 pg/ml and about 250 pg/ml, between about 50 pg/ml and about 200 pg/ml, between about 50 pg/ml and about 150 pg/ml, between about 50 pg/ml and about 100 pg/ml, between about 50 pg/ml and about 75 pg/ml, between about 100 pg/ml and about 1500 pg/ml, between about 100 pg/ml and about 1000 pg/ml, between about 100 pg/ml and about 800 pg/ml, between about 100 pg/ml and about 750 pg/ml, between about 100 pg/ml and about 500 pg/ml, between about 100 pg/ml and about 250 pg/ml, between about 100 pg/ml and about 200 pg/ml, between about 100 pg/ml and about 150 pg/ml, between about 100 pg/ml and about 125 pg/ml, between about 500 pg/ml and about 1500 pg/ml, between about 500 pg/ml and about 1000 pg/ml, between about 500 pg/ml and about 800 pg/ml, between about 500 pg/ml and about 750 pg/ml, between about 1000 pg/ml and about 1500 pg/ml, or between about 1000 pg/ml and about 1250 pg/ml. It should be appreciated, however, that other suitable markers (e.g., serum iron levels, total iron binding capacity (TIBC), ferritin levels, hemoglobin levels, hepatic iron content, Reticulocytes Hemoglobin Content, creatinine levels, etc.) may be evaluated to determine if the subject is suitable for method of treatment described herein.

In some embodiments, a subject in need of treatment in accordance with the disclosure has never received any therapeutic treatment for a hematologic disorder. In some embodiments, the subject in need is treated for MF-related anemia with any of the hepcidin antagonists described herein. In some embodiments, the hepcidin antagonist is a hemojuvelin-induced BMP signaling antagonist. In some embodiments, the hemojuvelin-induced BMP signaling antagonist is a BMP antagonist. In some embodiments, the BMP antagonist is a BMP2, BMP4, BMPS or BMP6 antagonist. In some embodiments, the BMP antagonist is BMP6 antagonist. In some embodiments, the hemojuvelin-induced BMP signaling antagonist is a BMP6 neutralizing antibody. In some embodiments, the BMP6 neutralizing antibody is LY311359, CSJ137, or KY1070.

In some embodiments, a subject in need of treatment in accordance with the disclosure is treated with a modified heparin selected from: SST0001, RO-82, RO-68, NAc-91, and NacRO-00.

In some embodiments, a subject in need of treatment in accordance with the disclosure is treated with a hemojuvelin (HJV) antagonist. In some embodiments, the HJV antagonist is an anti-HJV antibody (e.g., any of the anti-HJV antibody described in Table 1 or Table 2). In some embodiments, the HJV antagonist is HJV-35202. In some embodiments, the HJV antagonist is a soluble HJV. In some embodiments, the soluble HJV is a soluble hemojuvelin-Fc fusion protein. In some embodiments, the soluble HJV-Fc fusion protein is FMX8. In some embodiments, the HJV antagonist is any of the other HJV antagonists described herein.

In some embodiments, a subject in need of treatment in accordance with the disclosure is treated with a BMP receptor antagonist. In some embodiments, the BMP receptor antagonist is a BMP type I receptor antagonist. In some embodiments, the BMP type I receptor antagonist is an ALK2 antagonist. In some embodiments, the ALK2 antagonist is INCB000928, KER-047 or BLU-782. In some embodiments, the ALK2 inhibitor selectively inhibits its target molecule (e.g., ALK2) compared with a reference molecule (e.g., JAK1/2). In some embodiments, the reference molecule is JAK2. In some embodiments, the ALK2 inhibitor is not Momelotinib. In some embodiments, the ALK2 antagonist is any of the ALK2 antagonist described herein. In some embodiments, the ALK2 inhibitor is not a selective ALK2 inhibitor. In some embodiments, the ALK2 inhibitor also inhibits other target molecules (e.g., JAK1/2). In some embodiments, the ALK2 inhibitor is Momelotinib. In some embodiments, the BMP receptor antagonist is a BMP type II receptor antagonist. In some embodiments, the BMP type II receptor antagonist is an ActRIIA or ActRIIB antagonist. In some embodiments, the ActRIIA or ActRIIB antagonist is a GDF ligand trap. In some embodiments, the GDF ligand trap is sotatercept, or luspatercept. In some embodiments, the BMP receptor antagonist is any of the BMP receptor antagonist described herein.

In some embodiments, a subject in need of treatment in accordance with the disclosure is treated with a recombinant SMAD6 or SMAD7. In some embodiments, a subject in need of treatment in accordance with the disclosure is treated with an antagonist targeting SMAD1, SMAD4, SMAD5 and/or SMAD8 (e.g., intracellular antibodies or inhibitory nucleic acids targeting SMAD1, SMAD4, SMAD5 and/or SMAD8).

In some embodiments a subject in need of treatment in accordance with the disclosure is treated with a hepcidin neutralizing agent. In some embodiments, the hepcidin neutralizing agent is NOX-94, a PEGylated L-stereoisomer RNA aptamer that binds and neutralizes hepcidin. In some embodiments, the hepcidin neutralizing agent is PRS-080, an anticalin against hepcidin. In some embodiments, the hepcidin neutralizing agent is LY2787106, a monoclonal antibody targeting hepcidin. In some embodiments, the hepcidin neutralizing agent is any of the hepcidin neutralizing agent described herein.

In some embodiments, a subject in need of treatment in accordance with the disclosure continues to receive a therapeutic treatment for a hematologic disorder. The disclosure therefore provides, in some aspects, compositions and methods for treating ACD by administering to a subject in need thereof a In some embodiments, a subject is administered a hepcidin antagonist described herein in combination with one or more additional therapeutic agent (e.g., JAK-STAT inhibitors, GDF traps, a BET inhibitor or an immunomodulatory agent/erythropoietin stimulating agent). In some embodiments, the hepcidin antagonist to be used in combination therapy with one or more additional therapeutic agent (e.g., JAK-STAT inhibitors, GDF traps, a BET inhibitor or an immunomodulatory agent/erythropoietin stimulating agent) is an HJV induced BMP-signaling pathway antagonist such as BMP antagonist (e.g., BMP6 antagonist or modified heparins), BMP receptor antagonist (e.g., ALK2 antagonist), HJV antagonist (e.g., anti-HJV antibodies or soluble HJV such as soluble HJV. Fc fusion protein), or hepcidin neutralizing agent (e.g., anti-hepcidin antibody, anticalin targeting hepcidin, or inhibitory nucleic acid targeting hepcidin. In some embodiments, the administration of hepcidin antagonist described herein results in increased level of bio-available iron for erythropoiesis.

In some embodiments, a subject is administered a hepcidin antagonist (e.g., an HJV induced BMP-signaling pathway antagonist such as BMP antagonist (e.g., BMP6 antagonist or modified heparins), BMP receptor antagonist (e.g., ALK2 antagonist), HJV antagonist (e.g., anti-HJV antibodies or soluble HJV such as soluble HJV. Fc fusion protein), or hepcidin neutralizing agent (e.g., anti-hepcidin antibody, anticalin targeting hepcidin, or inhibitory nucleic acid targeting hepcidin)) in combination with immunomodulatory agent/erythropoietin stimulating agent (e.g., danazol, prednisone, thalidomide, lenalidomide, pomalidomide) or EPO. In some embodiments, the immunomodulatory agent/erythropoietin stimulating agent (e.g., danazol, prednisone, thalidomide, lenalidomide, pomalidomide) or EPO is administered in combination with an HJV-induced BMP signaling antagonist. In some embodiments, immunomodulatory agent/erythropoietin stimulating agent (e.g., danazol, prednisone, thalidomide, lenalidomide, pomalidomide) or EPO is combined with a BMP antagonist (e.g., BMP6 antagonist described herein). In some embodiments, the immunomodulatory agent/erythropoietin stimulating agent (e.g., danazol, prednisone, thalidomide, lenalidomide, pomalidomide) or EPO is combined with a HJV antagonist (e.g., HJV antagonist such as anti-HJV antibody, or soluble HJV.Fc fusion proteins). In some embodiments, immunomodulatory agent/erythropoietin stimulating agent (e.g., danazol, prednisone, thalidomide, lenalidomide, pomalidomide) or EPO is combined with an anti-HJV antibody described herein (e.g., any of the anti-HJV antibody listed in Table 1 or Table 2). In some embodiments, the HJV-Fc fusion protein is FMX8. In some embodiments, immunomodulatory agent/erythropoietin stimulating agent (e.g., danazol, prednisone, thalidomide, lenalidomide, pomalidomide) or EPO is combined with a BMP receptor antagonist (e.g., ALK2 inhibitor such as INCB000928, KER-047 or BLU-782 or GDF ligand trap described herein). In some embodiments, immunomodulatory agent/erythropoietin stimulating agent (e.g., danazol, prednisone, thalidomide, lenalidomide, pomalidomide) or EPO is combined with a hepcidin neutralizing agent (e.g., hepcidin neutralizing agent described herein). However, in some embodiments, immunomodulatory agent/erythropoietin stimulating agent (e.g., danazol, prednisone, thalidomide, lenalidomide, pomalidomide) or EPO is administered in combination with a JAK-STAT antagonist and/or any of the hepcidin antagonist described herein.

In some embodiments, a subject is administered a hepcidin antagonist (e.g., an HJV induced BMP-signaling pathway antagonist such as BMP antagonist (e.g., BMP6 antagonist or modified heparins), BMP receptor antagonist (e.g., ALK2 antagonist), HJV antagonist (e.g., anti-HJV antibodies or soluble HJV such as soluble HJV. Fc fusion protein), or hepcidin neutralizing agent (e.g., anti-hepcidin antibody, anticalin targeting hepcidin, or inhibitory nucleic acid targeting hepcidin)) in combination with a JAK-STAT pathway inhibitor. Any of the hepcidin antagonist described herein can be combined with a JAK-STAT inhibitor. In some embodiments, the JAK-STAT pathway inhibitor is a JAK inhibitor or a STAT inhibitor. In some embodiments, the JAK inhibitor is selective for one or both of subtypes JAK1 and JAK2 (e.g., ruxolitinib). In some embodiments, the JAK inhibitor is selective for JAK2 (e.g., fedratinib). In some embodiments, the STAT inhibitor is a STAT3 inhibitor. In some embodiments, the JAK inhibitor is not a selective JAK inhibitor. In some embodiments, the JAK inhibitor is an inhibitor for JAK1/2 and ACVRI/ALK2 (e.g., momelotinib). In some embodiments, the JAK1/2 or STAT3 inhibitor is selected from the group consisting of ruxolitinib, momelotinib, pacritinib, fedratinib, baricitinib, tofacitinib, oclacitinib, INCB039110, NSC13626, AG490, and PpYLKTK. In some embodiments, the JAK1/2 or STAT3 inhibitor is an IL6 antagonist or IL6R antagonist (e.g., IL6 or IL-6R antibodies). In some embodiments, a subject is administered with an HJV antagonist (e.g., anti-HJV antibody described herein, or a soluble HJV such as a soluble HJV.Fc fusion protein) in combination with JAK-STAT inhibitor (e.g., ruxolitinib, fedratinib, momelotinib, or IL6/IL6R antagonist). In some embodiments, a subject is administered with a BMP6 antagonist (e.g., anti-BMP6 antibodies) described herein in combination with JAK-STAT inhibitor (e.g., ruxolitinib, fedratinib, momelotinib, or IL6/IL6R antagonist). In some embodiments, a subject is administered with an ALK2 antagonist (e.g., anti-ALK2 antibodies or ALK2 inhibitors such as INCB000928, KER-047 or BLU-782) described herein in combination with JAK-STAT inhibitor (e.g., ruxolitinib, fedratinib, or IL6/IL6R antagonist). In some embodiments, a subject is administered with hepcidin neutralizing agent (e.g., anti-hepcidin antibodies) described herein in combination with JAK-STAT inhibitor (e.g., ruxolitinib, fedratinib, momelotinib, or IL6/IL6R antagonist).

In some embodiments, a hepcidin antagonist (e.g., an HJV induced BMP-signaling pathway antagonist such as BMP antagonist (e.g., BMP6 antagonist or modified heparins), BMP receptor antagonist (e.g., ALK2 antagonist), HJV antagonist (e.g., anti-HJV antibodies or soluble HJV such as soluble HJV. Fc fusion protein), or hepcidin neutralizing agent (e.g., anti-hepcidin antibody, anticalin targeting hepcidin, or inhibitory nucleic acid targeting hepcidin)) reduces the extent to which a subject exhibits an anemic response to the JAK-STAT pathway inhibitor. For example, in some embodiments, a subject treated with a JAK-STAT pathway inhibitor as a monotherapy may be characterized as having a deficiency in the ability of blood to transport oxygen as compared to the subject's pretreatment state, a deficiency in red blood cells as compared to the subject's pretreatment state, a deficiency in hemoglobin as compared to the subject's pretreatment state, an/or a deficiency in total blood volume as compared to the subject's pretreatment state. Accordingly, in some embodiments, the hepcidin antagonist (e.g., an HJV induced BMP-signaling pathway antagonist such as BMP antagonist (e.g., BMP6 antagonist or modified heparins), BMP receptor antagonist (e.g., ALK2 antagonist), HJV antagonist (e.g., anti-HJV antibodies or soluble HJV such as soluble HJV. Fc fusion protein), or hepcidin neutralizing agent (e.g., anti-hepcidin antibody, anticalin targeting hepcidin, or inhibitory nucleic acid targeting hepcidin)) reduces the extent to which a subject exhibits an anemic response to a JAK-STAT pathway inhibitor selected from the group consisting of ruxolitinib, pacritinib, fedratinib, baricitinib, tofacitinib, oclacitinib, INCB039110, NSC13626, AG490, and PpYLKTK. In some embodiments, the hepcidin antagonist (e.g., an HJV induced BMP-signaling pathway antagonist such as BMP antagonist (e.g., BMP6 antagonist or modified heparins), BMP receptor antagonist (e.g., ALK2 antagonist), HJV antagonist (e.g., anti-HJV antibodies or soluble HJV such as soluble HJV. Fc fusion protein), or hepcidin neutralizing agent (e.g., anti-hepcidin antibody, anticalin targeting hepcidin, or inhibitory nucleic acid targeting hepcidin)) reduces the extent to which a subject exhibits an anemic response to JAK-STAT inhibitor (e.g., ruxolitinib) administration.

In some embodiments, a subject is administered a hepcidin antagonist (e.g., an HJV induced BMP-signaling pathway antagonist such as BMP antagonist (e.g., BMP6 antagonist or modified heparins), BMP receptor antagonist (e.g., ALK2 antagonist), HJV antagonist (e.g., anti-HJV antibodies or soluble HJV such as soluble HJV. Fc fusion protein), or hepcidin neutralizing agent (e.g., anti-hepcidin antibody, anticalin targeting hepcidin, or inhibitory nucleic acid targeting hepcidin)) in combination with a growth factor ligand trap. In some embodiments, the growth factor ligand trap is a transforming growth factor beta (TGF-β) ligand trap. In some embodiments, the TGF-β ligand trap is sotatercept or luspatercept. In some embodiments, a subject is administered a hemojuvelin antagonist in combination with an anti-fibrotic agent. In some embodiments, the anti-fibrotic agent is PRM-151. In some embodiments, a growth factor ligand trap is administered in combination with an HJV-induced BMP signaling antagonist. In some embodiments, a growth factor ligand trap is combined with a BMP antagonist (e.g., BMP6 antagonist described herein). In some embodiments, a growth factor ligand trap is combined with a HJV antagonist (e.g., HJV antagonist such as anti-HJV antibody, or soluble HJV.Fc fusion proteins). In some embodiments, a growth factor ligand trap is combined with an anti-HJV antibody described herein (e.g., any of the anti-HJV antibody listed in Table 1 or Table 2). In some embodiments, the HJV-Fc fusion protein is FMX8. In some embodiments, a growth factor ligand trap is combined with a BMP antagonist (e.g., BMP6 antagonist described herein). In some embodiments, a growth factor ligand trap is combined with a BMP receptor antagonist (e.g., ALK2 inhibitor such as INCB000928, KER-047 or BLU-782 or GDF ligand trap described herein). In some embodiments, a growth factor ligand trap is combined with a hepcidin neutralizing agent (e.g., hepcidin neutralizing agent described herein). However, in some embodiments, a growth factor ligand trap is administered in combination with a JAK-STAT antagonist and/or any of the hepcidin antagonist described herein.

In some embodiments, the present disclosure provides a method of treating anemia in a subject having ACD using a combination of a hepcidin antagonist (e.g., an HJV induced BMP-signaling pathway antagonist such as BMP antagonist (e.g., BMP6 antagonist or modified heparins), BMP receptor antagonist (e.g., ALK2 antagonist), HJV antagonist (e.g., anti-HJV antibodies or soluble HJV such as soluble HJV. Fc fusion protein), or hepcidin neutralizing agent (e.g., anti-hepcidin antibody, anticalin targeting hepcidin, or inhibitory nucleic acid targeting hepcidin)) and a BET inhibitor (e.g., CPI-0610). In some embodiments, a BET inhibitor (e.g., CPI-0610) is administered in combination with an HJV-induced BMP signaling antagonist. In some embodiments, a BET inhibitor (e.g., CPI-0610) is combined with a BMP antagonist (e.g., BMP6 antagonist described herein). In some embodiments, BET inhibitor (e.g., CPI-0610) is combined with a HJV antagonist (e.g., HJV antagonist such as anti-HJV antibody, or soluble HJV.Fc fusion proteins). In some embodiments, a BET inhibitor (e.g., CPI-0610) is combined with an anti-HJV antibody described herein (e.g., any of the anti-HJV antibody listed in Table 1 or Table 2). In some embodiments, the HJV-Fc fusion protein is FMX8. In some embodiments, a BET inhibitor (e.g., CPI-0610) is combined with a BMP antagonist (e.g., BMP6 antagonist described herein). In some embodiments, a BET inhibitor (e.g., CPI-0610) is combined with a BMP receptor antagonist (e.g., ALK2 inhibitor such as INCB000928, KER-047 or BLU-782 or GDF ligand trap described herein). In some embodiments, a BET inhibitor (e.g., CPI-0610) is combined with a hepcidin neutralizing agent (e.g., hepcidin neutralizing agent described herein). However, in some embodiments, a BET inhibitor (e.g., CPI-0610) is administered in combination with a JAK-STAT antagonist and/or any of the hepcidin antagonist described herein.

Successful treatment of a subject in accordance with the disclosure may be determined by methods known in the art or by a skilled practitioner. In some embodiments, hepcidin antagonist treatment is evaluated based on serum hepcidin levels in a subject. For example, in some embodiments, baseline serum hepcidin levels in a subject are determined (e.g., before treatment with a hepcidin antagonist or otherwise in absence of hepcidin antagonist treatment at the time of determining) and compared to post-treatment serum hepcidin levels in the subject. In some embodiments, a subject is successfully treated where a hepcidin antagonist decreases serum hepcidin levels in the subject by between about 1 ng/mL and about 300 ng/mL. In some embodiments, the hepcidin antagonist decreases serum hepcidin levels in a subject by between about 1 ng/mL and about 200 ng/mL, between about 1 ng/mL and about 100 ng/mL, between about 1 ng/mL and about 50 ng/mL, between about 1 ng/mL and about 10 ng/mL, between about 10 ng/mL and about 100 ng/mL, or between about 10 ng/mL and about 50 ng/mL.

In some embodiments, hepcidin antagonist treatment is evaluated based on serum ferritin levels in a subject. For example, in some embodiments, baseline serum ferritin levels in a subject are determined (e.g., before treatment with a hepcidin antagonist or otherwise in absence of hepcidin antagonist treatment at the time of determining) and compared to post-treatment serum ferritin levels in the subject. In some embodiments, a subject is successfully treated where a hepcidin antagonist decreases serum ferritin levels in the subject by between about 1 ng/mL and about 200 ng/mL. In some embodiments, the hepcidin antagonist decreases serum ferritin levels in a subject by between about 1 ng/mL and about 100 ng/mL, between about 1 ng/mL and about 50 ng/mL, between about 1 ng/mL and about 25 ng/mL, between about 1 ng/mL and about 10 ng/mL, between about 10 ng/mL and about 100 ng/mL, or between about 10 ng/mL and about 50 ng/mL.

In some embodiments, hepcidin antagonist treatment is evaluated based on serum hemoglobin levels in a subject. For example, in some embodiments, baseline serum hemoglobin levels in a subject are determined (e.g., before treatment with a hepcidin antagonist or otherwise in absence of hepcidin antagonist treatment at the time of determining) and compared to post-treatment serum hemoglobin levels in the subject. In some embodiments, a subject is successfully treated where a hepcidin antagonist increases serum hemoglobin levels in the subject by between about 0.01 g/dL and about 5 g/dL. In some embodiments, the hepcidin antagonist decreases serum ferritin levels in a subject by between about 0.01 g/dL and about 1 g/dL, between about 0.1 g/dL and about 5 g/dL, between about 1 g/dL and about 5 g/dL, between about 0.01 g/dL and about 0.1 g/dL, between about 0.5 g/dL and about 2.5 g/dL, or between about 0.1 g/dL and about 1 g/dL.

Determination of whether an amount of the hepcidin antagonist achieved the therapeutic effect would be evident to one of skill in the art based on the teachings provided herein. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. The particular dosage regimen, i.e., dose, timing and repetition, used in the method described herein will depend on the particular subject and that subject's medical history, as discussed herein.

Empirical considerations, such as time to maximum effect, half-life, and/or time above a specific concentration generally will contribute to the determination of the dosage.

In some embodiments, dosages for a hepcidin antagonist as described herein may be determined empirically in individuals who have been given one or more administration(s) of the antibody. Individuals are given incremental dosages of the antagonist. To assess efficacy of the antagonist, an indicator of the disease/disorder can be followed.

Dosing frequencies may vary in accordance with the claimed methods. In some embodiments, a composition will be administered once. In some embodiments, a treatment will be administered on multiple occasions. In some embodiments, dosing frequency is every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. In some embodiments, a composition will be administered daily, biweekly, weekly, bimonthly, monthly, or at any time interval that provide suitable (e.g., maximal) efficacy while minimizing safety risks to the subject. Generally, the efficacy and the treatment and safety risks may be monitored throughout the course of treatment.

In some embodiments, administration of hepcidin antagonist results in a decrease in serum hepcidin-25 concentration and/or increase serum TSAT %, and in some embodiments, these effects persist for a period of time (e.g., one month or more). Accordingly, in some embodiments, timing and frequency of administration of hepcidin antagonist can be determined by monitoring one or more biomarkers, e.g., criteria to assess iron availability or flag possible iron overload. For example, in some embodiments, hepcidin antagonist is administered intermittently or in accordance with the level of a particular biomarker such as serum hepcidin-25 levels or transferrin saturation percentage (TSAT %). In some embodiments, a biomarker level described herein can be used to determine whether a subject is a candidate for treatment. However, in some embodiments, a biomarker may be used to determine whether to continue treatment or to resume a treatment or to halt a treatment, e.g., with a hepcidin antagonist.

For example, in some embodiments, a subject may be considered as not being a candidate for treatment if TSAT % of the subject is at or above 70%, at or above 75%, at or above 80%, at or above 85%, at or above 90%, or at or above 95%. In some cases, if TSAT % of the subject is at or above 70%, at or above 75%, at or above 80%, at or above 85%, at or above 90%, or at or above 95%, an ongoing treatment with a hepcidin antagonist may be stopped or temporarily stopped, e.g., to prevent iron overload. In other embodiments, administration of an anti-HJV antibody may be performed when a TSAT % of a subject is at or below 95%, at or below 90%, at or below 80%, at or below 70%, at or below 65%, at or below 60%, at or below 55%, at or below 50%, at or below 45%, at or below 40%, at or below 35%, or at or below 30%. Thus, in some embodiments, TSAT % of a subject can be monitored, e.g., continuously or periodically, while a patient is receiving a treatment or under care of a treating physician, e.g., for anemia, to prevent iron overload or otherwise to assess whether further treatments are appropriate. It should be appreciated, however, that other suitable markers may be monitored to determine dosage and dosage frequency (including, for example, ferritin levels, serum iron levels, creatinine levels, etc.) in accordance with the methods provided herein.

In some embodiments, a subject may be administered a composition provided herein (e.g., hepcidin antagonist) at one or more intervals during a set period of time. In some cases, periods of time during which a subject is administered a composition at one or more intervals may be separated by periods of time in which the subject is not administered the composition. In some embodiments, the relative durations of respective periods of time may depend on the subject's response to treatment or severity of disease or both and/or may be determined based on the judgment of a treating physician. For example, in some embodiments, during the course of a year a subject may be administered a composition weekly, biweekly or monthly for two months and then the administration is stopped for ten months. In some embodiments, during the course of a year a subject may be administered a composition weekly, biweekly or monthly for three months and then the administration is stopped for nine months. In some embodiments, during the course of a year a subject may be administered a composition weekly, biweekly or monthly for four months and then the administration is stopped for eight months. In some embodiments, during the course of a year a subject may be administered a composition weekly, biweekly or monthly for five months and then the administration is stopped for seven months. In some embodiments, during the course of a year a subject may be administered a composition weekly, biweekly or monthly for six months and then the administration is stopped for six months. In some embodiments, during the course of a year a subject may be administered a composition weekly, biweekly or monthly for seven months and then the administration is stopped for five months. In some embodiments, during the course of a year a subject may be administered a composition weekly, biweekly or monthly for eight months and then the administration is stopped for four months. In some embodiments, during the course of a year a subject may be administered a composition weekly, biweekly or monthly for nine months and then the administration is stopped for three months. In some embodiments, during the course of a year a subject may be administered a composition weekly, biweekly or monthly for ten months and then the administration is stopped for two months. In some embodiments, during the course of a year a subject may be administered a composition weekly, biweekly or monthly for two months on, two months off; or for three months on, three months off; or for four months on, four months off.

In some embodiments, a hepcidin antagonist can be administered parenterally. For example, a parenterally administered composition may be administered by subcutaneous, intracutaneous, intravenous, intraperitoneal, intratumor, intramuscular, intraarticular, intraarterial, or infusion techniques. In addition, it can be administered to the subject via injectable depot routes of administration such as using 1-, 3-, or 6-month depot injectable or biodegradable materials and methods. In some embodiments, the hepcidin antagonist is administered subcutaneously. In some embodiments, the hepcidin antagonist is administered intravenously.

### EXAMPLES

### Example 1: Treating Anemia of Chronic Diseases with Hepcidin Antagonists

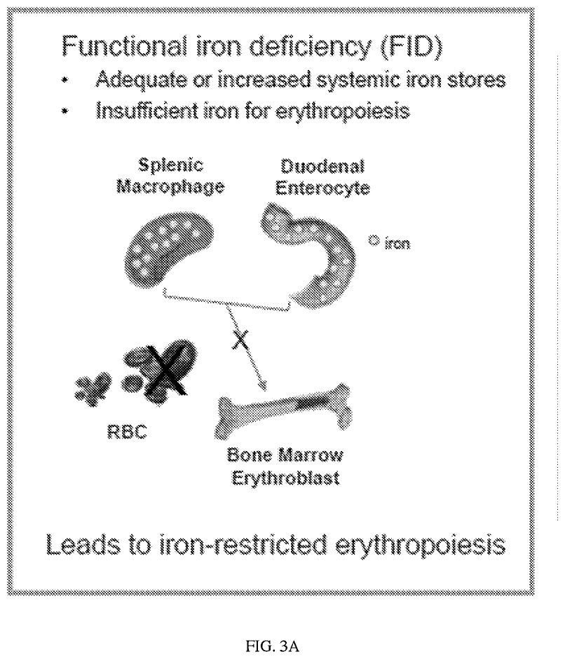

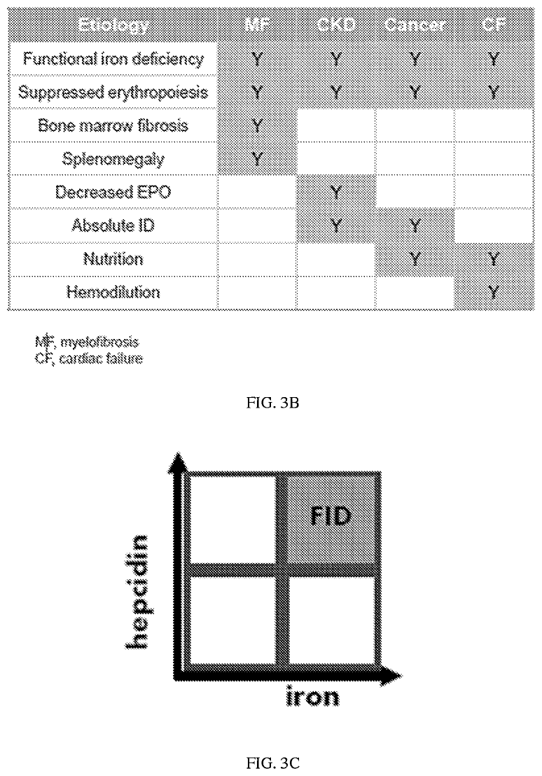

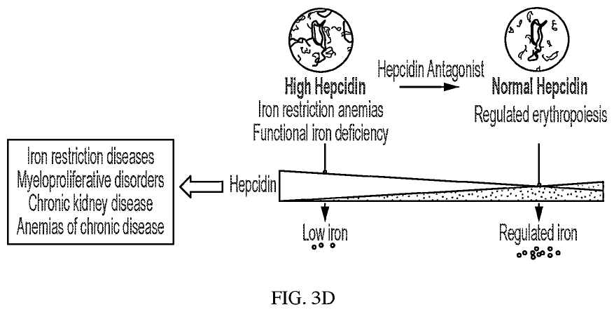

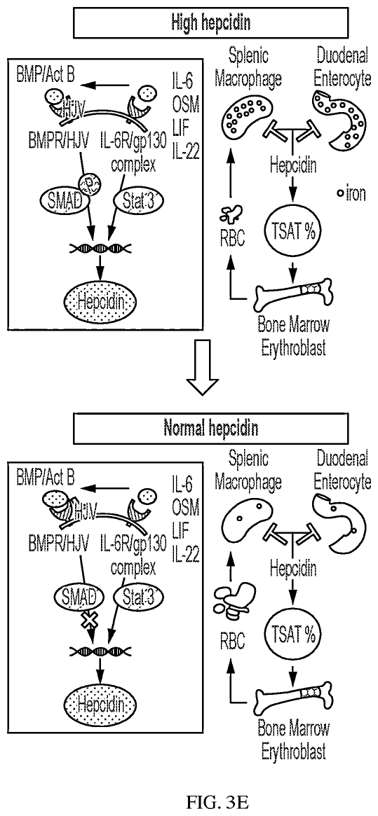

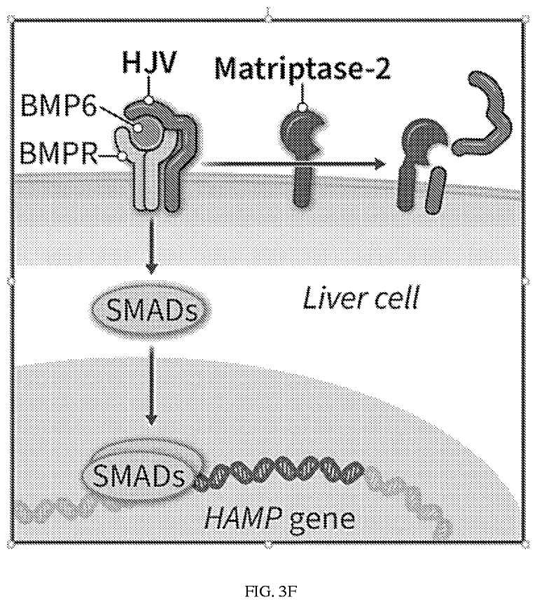

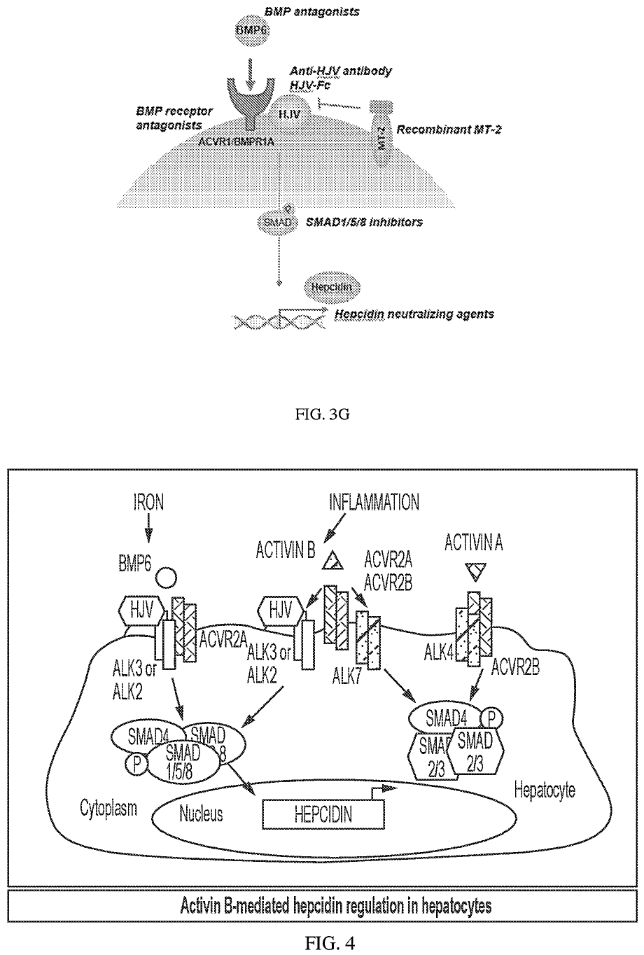

Iron-restricted erythropoiesis occurs in cases of both absolute and functional iron deficiency (FID). FID represents a state of iron-restricted erythropoiesis characterized by an imbalance between iron demand and serum iron that is readily available for effective erythropoiesis. In FID, even when the body has Adequate or increased systemic iron stores, iron is sequestered and not available for erythropoiesis (FIG. 3A). It has been shown that FID is caused by an increase of hepcidin relative to the iron store levels. Increased hepcidin is observed in diseases associated with FID, such as inflammation (e.g., ACD, chronic kidney disease on hemodialysis (CKD-HD), autoimmunity, etc.), iron overload (e.g., CKD). genetic diseases (e.g., iron-refractory iron deficiency anemia (IRIDA)), uremic toxins (e.g., CKD), decreased clearance (e.g., chronic kidney disease on peritoneal dialysis CKD-PD)), and cancer.

FID is a common feature of the anemia of inflammation and chronic diseases (AI/ACD), regardless of etiology of the disease. Contribution of FID to anemia varies between diseases and patients with same disease (AI/ACD has different etiological factors) (FIG. 3B). The shared feature among different etiology of FID is increased hepcidin level and adequate to increased iron storage (FIG. 3C).

The present disclosure, at least in part, is sought to decrease hepcidin level to restore normal erythropoiesis in patients with FID (e.g., ACD patients) using hepcidin antagonists.

The HAMP gene encodes hepcidin precursor protein, which is primarily expressed by hepatocytes in the liver, and at lower levels by other cells in extrahepatic tissues. The precursor protein is subsequently cleaved to yield bioactive hepcidin. Transcriptional regulators of HAMP gene include BMP signaling and JAK-STAT3 signaling. Hemojuvelin (HJV) is an important co-receptor in inducing hepcidin expression by BMP signaling. Inhibition of HJV-induced BMP signaling pathway and/or JAK-STAT3 signaling pathway by targeting any component of these pathways can lead to decrease of hepcidin expression. To decrease hepcidin level by targeting the HJV-induced BMP signaling pathway, BMP antagonists (e.g., BMP6 antagonists), HJV antagonists (e.g., anti-HJV antibody, HJV-Fc), BMP receptor antagonists, SMAD1/5/8 antagonists, or hepcidin neutralizing agent can be employed (FIG. 3G).

For example, anti-HJV antibodies have been shown to be able to reduce hepcidin synthesis and reduce anemia severity (Kovacs et al., Anti-hemojuvelin antibody corrects anemia caused by inappropriately high hepcidin levels, Haematologica. 2016 May; 101(5): e173-e176). FIG. 3E. In addition, HJV is regulated by matripatase-2. Matriptase-2, encoded by the TMPRSS6 gene, is a member of the type II transmembrane serine protease family. Matriptase-2 has been established to be essential in iron homeostasis. TMPRSS6 is expressed mainly in the liver and negatively regulates the production of hepcidin by cleaving the membrane bound hemojvelin (e.g., Du X., et al. (2008). The serine protease TMPRSS6 is required to sense iron deficiency. Science 320 1088-1092) (FIG. 3F). Therefore, increasing matriptase-2 expression in liver cells may be another method to negatively regulate hepcidin expression.

Further, it has been shown that activin B is capable of stimulating SMAD1/5/8 signaling and hepcidin expression in liver cells to a similar degree as canonical SMAD2/3 signaling, and with similar or modestly reduced potency compared with BMP6. Activin B stimulates hepcidin via classical activin type II receptors ACVR2A and ACVR2B, non-canonical BMP type I receptors activin receptor-like kinase 2 and activin receptor-like kinase 3, and SMADS. The co-receptor hemojuvelin binds to activin B and facilitates activin B-SMAD1/5/8 signaling. (Canali et al., Activin B Induces Noncanonical SMAD1/5/8 Signaling via BMP Type I Receptors in Hepatocytes: Evidence for a Role in Hepcidin Induction by Inflammation in Male Mice, Endocrinology. 2016 March; 157(3): 1146-1162). FIG. 4 illustrates how Activin B mediates hepcidin regulation in hepatocytes.

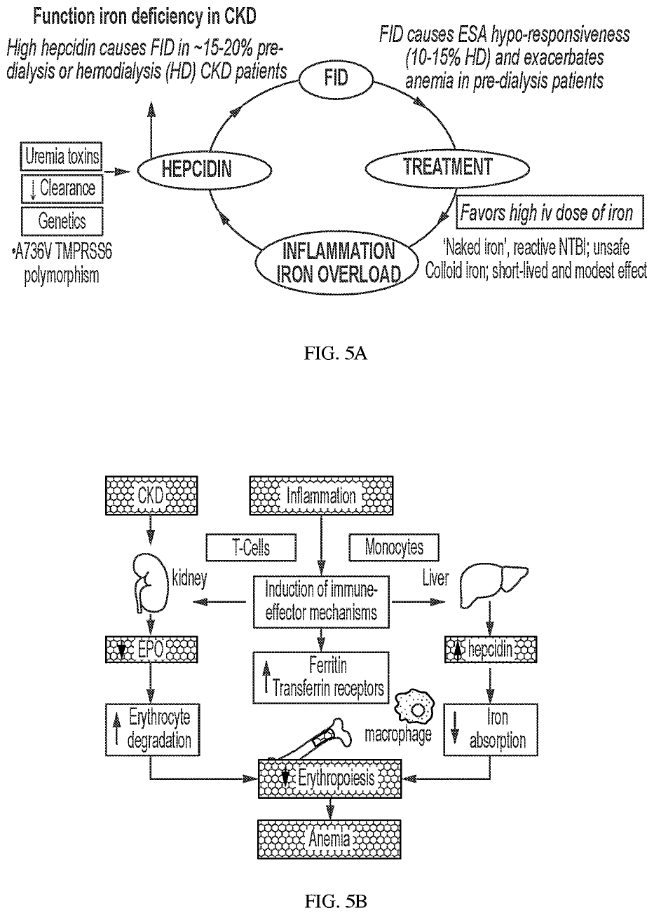

Chronic kidney disease (CKD), which leads to end stage renal disease, is one of the major causes for ACD. Functional iron deficiency is common in CKD patients—about 20-40% CDK patients are reported to have FID. FID in CKD is correlated with anemia severity and disease severity, and is also negatively correlated with survival. The prevalence of anemia in CKD progressively increases when eGFR<60 mL/min/1.73 m2. Anemia appears as early as stage 3 CKD, and is most frequent in stage 4. In CKD, iron deficiency anemia, both absolute and functional, are common complications. Such patients are often treated with high doses of intravenous iron. High dose iron treatment not only does not correctly address the underlying cause of FID, but also results in a vicious cycle of impaired iron metabolism in CKD patient (FIG. 5A). High does intravenous iron leads to inflammation and iron overload, which in turn increases hepcidin expression. At the same time, conditions associated with CKD, such as uremia toxins, reduced clearance, and certain genetic factors (e.g., A736V polymorphism in TMPRSS6 gene), further increase hepcidin level in the patients. High hepcidin level causes FID and anemic symptoms in CKD patients. FID causes erythropoietin stimulating agent (ESA) hypo-responsiveness and exacerbates anemia, which leads to further high does intravenous iron treatment, thereby completing the viscous cycle. In CKD patients, FID is defined as TSAT≤20% and elevated serum ferritin as high as 800 ng/mL (Locatelly et al, Kidney Disease: Improving Global Outcomes Guidelines on Anaemia Management in Chronic Kidney Disease: A European Renal Best Practice Position Statement, Nephrol Dial Transplant, 2013 June; 28(6):1346-59; Capellini et al, Iron deficiency across chronic inflammatory conditions: International expert opinion on definition, diagnosis, and management, Am J Hematol. 2017 October; 92(10): 1068-1078). In present disclosure, FID can be defined as TSAT≤20% and ferritin >200 ng/mL.

Anemia is proposed to also contribute to the deterioration of kidney function. Anemia of CKD leads to hypoxia and proteinuria. Signs and symptoms include shortness of breath, paleness, palpitations, and fatigue. Kidney hypoxia activates RAAS, leading to renal vasoconstriction and proteinuria (in diabetic nephropathy) (O'Mara, 2008). Kidney hypoxia should stimulates the production of erythropoietin (EPO), but in damaged kidney, EPO production is compromised. Further, high hepcidin level decreases ferroportin levels, decreasing iron absorption and consequently erythropoiesis. Proinflammatory cytokines, such as interleukins (IL-1 and IL-6) and TNF-α, destroy RBC precursors and reduce the number of erythropoietin receptors on progenitor cells. Additional contributors to anemia associated with CKD include blood loss, dietary iron, vitB12 or folic acid deficiency, and bone marrow suppression. FIG. 5B describes the pathophysiology of renal anemia.

Accordingly, the present disclosure, provides a method for treating anemia associated with chronic diseases (e.g., CKD) with an increased hepcidin level. Reducing hepcidin level using the hepcidin antagonists described herein, and optionally in combination with additional therapeutic agents (e.g., immunomodulatory agent/erythropoietin stimulating agents, GDF trap, BET inhibitors, and/or JAK-STAT inhibitors) corrects the functional and/or absolute iron deficiency in these patients, thereby improving disease conditions and survival.

### Example 2: Anti-HJV Antibody Decreases IL-6 Induced Hepcidin Expression in Non-Human Primates

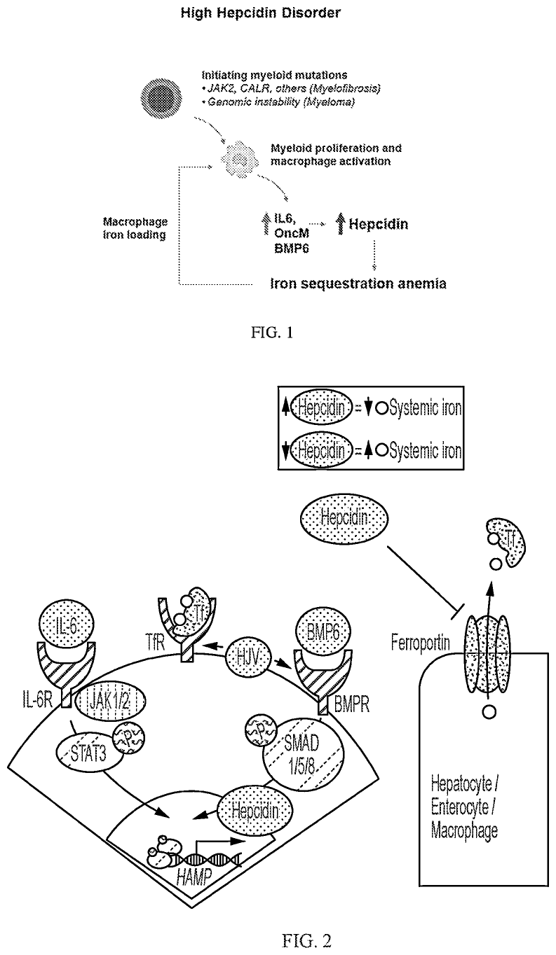

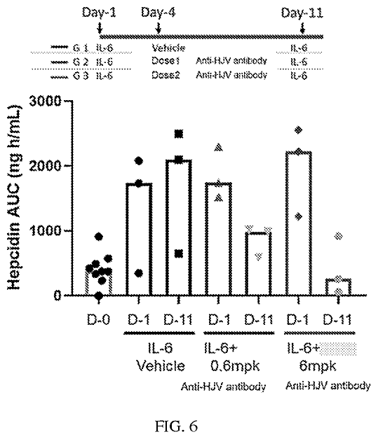

As depict in FIG. 1, in high hepcidin disorders such as anemia of chronic disease, pro-inflammatory cytokines that induce hepcidin synthesis, such as IL-6 and oncostatin-M, are typically increased and associated with iron sequestration, macrophage iron loading, as well as myeloid proliferation and macrophage activation. To test whether IL-6 indeed increases hepcidin expression and whether anti-HJV antibody is capable of inhibiting hepcidin expression induced by IL-6 in non-human primates, cynos were challenged with IL-6 on day 1, and divided into three groups. On day 4, cynos in Group 1 received vehicle control, cynos in Group 2 received an anti-HJV antibody (CDR-H1: SEQ ID NO: 1, CDR-H2: SEQ ID NO: 2, CDR-H3: SEQ ID NO: 3, CDR-L1: SEQ ID NO: 7, CDR-L2: SEQ ID NO: 8, and CDR-L3: SEQ ID NO: 9) at 0.6 mg/kg, and cynos in Group 3 received the same anti-HJV antibody at 6.0 mg/kg. On day 11, cynos in all three groups were challenged with IL-6 again, and plasma hepcidin-25 in all cynos was measured. As shown in FIG. 6, IL-6 challenge increased plasma hepcidin-25 concentrations on Day 1, compared to pre-challenge baseline (BL) in all three groups of cynos. After the second IL-6 challenge on Day 11, cynos in group 1 showed an increase in plasma hepcidin-25 similar to that observed on Day 1. However, for cynos in groups 2 (0.6 mg/kg anti-HJV antibody) and 3 (6 mg/kg anti-HJV antibody), the presence the anti-HJV antibody prevented the IL-6 induced increase in plasma hepcidin-25 on Day 11 in a dose-dependent manner. That is, anti-HJV antibody was effective in preventing inflammation-induced (IL 6) hepcidin increase in a dose-dependent manner in cynos. These results suggest that anti-HJV antibody are capable of inhibiting hepcidin expression induced by the IL-6 signaling pathway.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A method of treating a subject having an anemia of chronic disease, the method comprising:
administering to the subject an effective amount of a hepcidin antagonist.

2. The method of claim 1, wherein the subject has impaired iron availability/functional iron deficiency.

3. The method of claim 1 or 2, wherein the hepcidin antagonist is a hemojuvelin-induced BMP signaling antagonist.

4. The method of claim 3, wherein the hemojuvelin-induced BMP signaling antagonist is a BMP antagonist.

5. The method of claim 4, wherein the BMP antagonist is a BMP2, BMP4, BMP5 or BMP6 antagonist.

6. The method of claim 5, wherein the BMP antagonist is BMP6 antagonist.

7. The method of any one of claims 3-6, wherein the hemojuvelin-induced BMP signaling antagonist selectively inhibits its target molecule.

8. The method of claim 7, wherein the target molecule is a BMP receptor.

9. The method of claim 7 or 8, wherein the hemojuvelin-induced BMP signaling antagonist selectively inhibits its target molecule compared with a reference molecule.

10. The method of claim 9, wherein the reference molecule is JAK2.

11. The method of claim 10, wherein the hemojuvelin-induced BMP signaling antagonist selectively inhibits its target molecule compared with the reference molecule, such that it has an half maximal inhibitory concentration (IC₅₀) for the reference molecule that is at least 10-fold higher (e.g., in the range of 10¹ to 10⁶-fold higher) than the IC₅₀ for the target molecule, as measured in a kinase potency assay.

12. The method of any one of claims 1-4, wherein the Hemojuvelin-induced BMP signaling antagonist is sHJV or a soluble hemojuvelin-Fc fusion protein.

13. The method of claim 12, wherein the soluble HJV-Fc fusion protein is FMX8.

14. The method of claim 4, wherein the hemojuvelin-induced BMP signaling antagonist is a BMP6 neutralizing antibody.

15. The method of claim 14, wherein the BMP6 neutralizing antibody is LY311359, CSJ137, or KY1070.

16. The method of claim 3 or 4, wherein hemojuvelin-induced BMP signaling is a modified heparin selected from: SST0001, RO-82, RO-68, NAc-91, and NacRO-00.

17. The method of claim 3 or 4, wherein the Hemojuvelin-induced BMP signaling antagonist is recombinant SMAD6 or SMAD7.

18. The method of claim 1, wherein the hepcidin antagonist is a hepcidin neutralizing agent.

19. The method of claim 18, wherein the hepcidin neutralizing agent is NOX-94, a PEGylated L-stereoisomer RNA aptamer that binds and neutralizes hepcidin.

20. The method of claim 18, wherein the hepcidin neutralizing agent is PRS-080, an anticalin against hepcidin.

21. The method of claim 18, wherein the hepcidin neutralizing agent is LY2787106, a monoclonal antibody targeting hepcidin.

22. The method of any one of claim 3 or 4, wherein the hemojuvelin-induced BMP signaling antagonist is an ALK2 antagonist.

23. The method of claim 22, wherein the ALK2 antagonist is INCB000928, KER-047 or BLU-782.

24. The method of any one of claims 1 to 4, wherein the hepcidin antagonist is a hemojuvelin antagonist.

25. The method of claim 24, wherein the hemojuvelin antagonist is an anti-hemojuvelin antibody.

26. The method of claim 25, wherein the anti-hemojuvelin antibody preferentially binds RGMc versus RGMa and RGMb.

27. The method of claim 26, wherein the anti-hemojuvelin antibody binds RGMc with an equilibrium dissociation constant (K_{D}) less than 100 nM.

28. The method of claim 27, wherein the anti-HJV antibody is HJV-35202.

29. The method of claim 27, wherein the anti-HJV antibody is an anti-HJV antibody in Table 1.

30. The method of claim 29, wherein the anti-hemojuvelin antibody comprises:
(a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or
(b) a variable light chain region comprising a CDR1 comprising an amino acid sequence of SEQ ID NO: 4, a CDR2 comprising an amino acid sequence of SEQ ID NO: 5, and a CDR3 comprising an amino acid sequence of SEQ ID NO: 6.

31. The method of claim 29, wherein the anti-hemojuvelin antibody comprises:
(a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or
(b) a variable light chain region comprising a CDR1 comprising an amino acid sequence of SEQ ID NO: 7, a CDR2 comprising an amino acid sequence of SEQ ID NO: 8, and a CDR3 comprising an amino acid sequence of SEQ ID NO: 9.

32. The method of claim 29, wherein the anti-hemojuvelin antibody comprises:
(a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or
(b) a variable light chain region comprising a CDR1 comprising an amino acid sequence of SEQ ID NO: 10, a CDR2 comprising an amino acid sequence of SEQ ID NO: 11, and a CDR3 comprising an amino acid sequence of SEQ ID NO: 12.

33. The method of claim 29, wherein the anti-hemojuvelin antibody comprises:
(a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or
(b) a variable light chain region comprising a CDR1 comprising an amino acid sequence of SEQ ID NO: 13, a CDR2 comprising an amino acid sequence of SEQ ID NO: 14, and a CDR3 comprising an amino acid sequence of SEQ ID NO: 15.

34. The method of claim 29, wherein the anti-hemojuvelin antibody comprises:
(a) a variable heavy chain region comprising a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21; and/or
(b) a variable light chain region comprising a CDR1 comprising an amino acid sequence of SEQ ID NO: 22, a CDR2 comprising an amino acid sequence of SEQ ID NO: 23, and a CDR3 comprising an amino acid sequence of SEQ ID NO: 24.

35. The method of any one of claims 1-34, wherein the subject is erythrocyte transfusion-dependent.

36. The method of any one of claims 1-35, wherein the subject is erythrocyte transfusion-independent.

37. The method of any one of claims 1-36, wherein the anemia of chronic disease is an iron-restricted anemia.

38. The method of claim 37, wherein the iron-restricted anemia is associated with a functional iron deficiency.

39. The method of any one of claims 1-38, wherein the anemia of chronic disease is associated with (or caused by) chronic kidney disease.

40. The method of any one of claims 1-38, wherein the anemia of chronic disease is associated with (or caused by) cancer.

41. The method of claim 40, wherein the cancer is a myeloma.

42. The method of any one of claims 1-38, wherein the anemia of chronic disease is associated with (or caused by) a chronic infection.

43. The method of claim 42, wherein the infection is a bacterial, viral, fungal or parasitic infection.

44. The method of any one of claims 1-38, wherein the anemia of chronic disease is associated with (or caused by) an autoimmune disease.

45. The method of any one of claims 1-38, wherein the anemia of chronic disease is associated with (or caused by) a chronic disease that involves inflammation, such as inflammatory bowel disease, diabetes or heart failure.

46. The method of any one of claims 1-45, wherein the subject is identified prior to the treatment as having high hepcidin levels.

47. The method of any one of claims 1-46, wherein the subject is identified as having a functional iron deficiency.

48. The method of any one of claims 1-47, wherein the subject is identified as exhibiting inflammation and/or iron-restricted erythropoiesis.

49. The method of any one of claims 1-48, wherein the subject is a human.

50. The method of any one of claims 1-49, wherein the subject does not have a nutritional iron deficiency.

51. The method of any one of claims 1-50, wherein the subject has ferritin levels above 100 μg/L.

52. The method of any one of claims 1-51, wherein the subject has reticulocytes hemoglobin content less than 26 μg/cell.

53. The method of any one of claims 1-52, wherein the subject has a transferrin saturation level less than 50%.

54. The method of any one of claims 1-53, wherein the subject has hepatic iron levels higher than 2000 μg/g dry weight.

55. The method of any one of claims 1-54, wherein the subject has serum iron levels in a range of less than 50 μg/dL.

56. The method of any one of claims 1-55, wherein the subject has a total iron binding capacity in a range of less than 400 μg/dL.

57. The method of any one of claims 1-56, wherein the subject has hepcidin levels in a range of more than 55 ng/ml.

58. The method of any one of claims 1-57, wherein the subject has IL-6 levels of more than 1.8 pg/mL.

59. The method of any one of claims 1-58, wherein the subject has serum creatinine values of more than 2 mg/dL.

60. The method of any one of claims 1-59, wherein the subject has been identified as having hemoglobin levels in the range of 1.5 to 2.0 g/dL or 2.0 to 4.0 g/dL or more below normal hemoglobin levels.

61. The method of any one of claims 1-60, wherein the subject presents with a serum hemoglobin level of less than 10 g/dL.

62. The method of any one of claims 1-61, wherein the subject presents with a serum hemoglobin level of less than 8 g/dL.

63. The method of any one of claims 1-62, wherein the administration of the hepcidin antagonist increases hemoglobin level at least 1 g/dL from baseline.

64. The method of any one of claims 1-63, wherein the subject presents with thrombocytopenia, anemia, and/or neutropenia.

65. The method of any one of claims 1-64, wherein the subject has received one or more transfusions.

66. The method of claim 65, wherein the subject has transfusion-dependent anemia.

67. The method of claim 66, wherein the subject has received multiple transfusions over a twelve week period.

68. The method of any one of claims 1-67, wherein the subject has previous received one or more administrations of a JAK/STAT antagonist as a treatment for a chronic diseases.

69. The method of claim 68, wherein the subject received treatment with the JAK/STAT antagonist for 2-6 weeks.

70. The method of claim 68 or 69, wherein the JAK/STAT antagonist is selective for JAK1 or JAK2.

71. The method of any one of claims 68-70, wherein the JAK/STAT antagonist is not active against ACVR1/ALK2.

72. The method of any one of claims 68-71, wherein the JAK/STAT antagonist is ruxolitinib, fedratinib, pacritinib, baricitinib, tofacitinib, oclacitinib, or NSC13626.

73. The method of any one of claims 68-72, wherein the JAK/STAT antagonist inhibits IL6 mediated STAT3 activation.

74. The method of any one of claims 68-70, wherein JAK/STAT antagonist is GS-0387 or CYT-387.

75. The method of any one of claims 1-74, further comprising administering the subject with one or more additional therapeutic agents.

76. The method of claim 75, wherein the additional therapeutic agent is selected from a GDF trap, a Bromodomain and extra-terminal domain (BET) inhibitor, an erythropoiesis stimulating agent, or an immunomodulatory agent/erythropoietin stimulating agent.

77. The method of claim 76, wherein the GDF trap is sotatercept, luspatercept or KER-050.

78. The method of claim 76, wherein the BET inhibitor is CPI-0610.

79. The method of claim 76, wherein the immunomodulatory agent/erythropoietin stimulating agent is Pomalidomide.

80. The method of claim 76, wherein the erythropoiesis stimulating agent is Erythropoietin (EPO).

81. A method of treating a subject having an anemia of chronic disease, the method comprising:
the method comprising administering to the subject an effective amount of a hepcidin antagonist, and one or more additional therapeutic agent.

82. The method of claim 81, wherein the hepcidin antagonist is a HJV-induced BMP signaling antagonist, or a hepcidin neutralizing agent.

83. The method of claim 82, wherein the HJV-induced BMP signaling antagonist is a BMP antagonist, a HJV antagonist, a modified heparin targeting BMP6, or a recombinant SMAD6 or SMAD7.

84. The method of claim 83, wherein the BMP antagonist is a BMP6 neutralizing antibody selected from LY311359, CSJ137, and KY1070.

85. The method of claim 82, wherein the HJV-induced BMP signaling antagonist is an HJV antagonist.

86. The method of claim 85, wherein the HJV antagonist is an anti-HJV antibody.

87. The method of any one of claims 81-86, wherein the additional therapeutic agent is selected from a GDF trap, a JAK/STAT inhibitor, a BET inhibitor, erythropoiesis stimulating agent, or an immunomodulatory agent/erythropoietin stimulating agent.

88. The method of claim 87, wherein the additional therapeutic agent is the GDF trap.

89. The method of claim 88, wherein the GDF trap is sotatercept, luspatercept or KER-050.

90. The method of claim 87, wherein the JAK/STAT inhibitor is ruxolitinib, fedratinib, pacritinib, baricitinib, tofacitinib, oclacitinib, NSC13626 or momelotinib.

91. The method of claim 87, wherein the BET inhibitor is CPI-0610.

92. The method of claim 87, wherein the immunomodulatory agent is pomalidomide.

93. The method of claim 87, wherein the erythropoiesis stimulating agent is erythropoietin (EPO).

94. A method of treating a subject having or at risk of having an adverse reaction to a JAK-STAT antagonist, the method comprising: administering to the subject an effective amount of hemojuvelin-induced BMP signaling antagonist.


SEQUENCE LISTING (36 sequences)

SEQ ID NO: 1 - protein, 5 aa, Artificial Sequence
  Synthetic Polypeptide
NYGMN

SEQ ID NO: 2 - protein, 17 aa, Artificial Sequence
  Synthetic Polypeptide
MIYYDSSEKHYADSVKG

SEQ ID NO: 3 - protein, 6 aa, Artificial Sequence
  Synthetic Polypeptide
GTTPDY

SEQ ID NO: 4 - protein, 16 aa, Artificial Sequence
  Synthetic Polypeptide
RSSQSLESSDGDTFLE

SEQ ID NO: 5 - protein, 7 aa, Artificial Sequence
  Synthetic Polypeptide
DVSTRFS

SEQ ID NO: 6 - protein, 9 aa, Artificial Sequence
  Synthetic Polypeptide
FQVTHDPVT

SEQ ID NO: 7 - protein, 16 aa, Artificial Sequence
  Synthetic Polypeptide
RSSQSLEESDGYTFLH

SEQ ID NO: 8 - protein, 7 aa, Artificial Sequence
  Synthetic Polypeptide
EVSTRFS

SEQ ID NO: 9 - protein, 9 aa, Artificial Sequence
  Synthetic Polypeptide
FQATHDPLT

SEQ ID NO: 10 - protein, 16 aa, Artificial Sequence
  Synthetic Polypeptide
RSSQSLADSDGDTFLH

SEQ ID NO: 11 - protein, 7 aa, Artificial Sequence
  Synthetic Polypeptide
AVSHRFS

SEQ ID NO: 12 - protein, 9 aa, Artificial Sequence
  Synthetic Polypeptide
FQATHDPVT

SEQ ID NO: 13 - protein, 16 aa, Artificial Sequence
  Synthetic Polypeptide
RSSQSLEDSDGGTFLE

SEQ ID NO: 14 - protein, 7 aa, Artificial Sequence
  Synthetic Polypeptide
DVSSRFS

SEQ ID NO: 15 - protein, 9 aa, Artificial Sequence
  Synthetic Polypeptide
FQATHDPLS

SEQ ID NO: 16 - protein, 16 aa, Artificial Sequence
  Synthetic Polypeptide
RSSQSLEYSDGYTFLE

SEQ ID NO: 17 - protein, 7 aa, Artificial Sequence
  Synthetic Polypeptide
EVSNRFS

SEQ ID NO: 18 - protein, 9 aa, Artificial Sequence
  Synthetic Polypeptide
FQATHDPLT

SEQ ID NO: 19 - protein, 10 aa, Artificial Sequence
  Synthetic Polypeptide
GFNIRDFYIH

SEQ ID NO: 20 - protein, 17 aa, Artificial Sequence
  Synthetic Polypeptide
WIDPENGDIEYAPKFQG

SEQ ID NO: 21 - protein, 7 aa, Artificial Sequence
  Synthetic Polypeptide
NGYYLDY

SEQ ID NO: 22 - protein, 16 aa, Artificial Sequence
  Synthetic Polypeptide
KSGQSLLHSDGKTYLN

SEQ ID NO: 23 - protein, 7 aa, Artificial Sequence
  Synthetic Polypeptide
LVSKLDS

SEQ ID NO: 24 - protein, 9 aa, Artificial Sequence
  Synthetic Polypeptide
WQGTHSPWT

SEQ ID NO: 25 - protein, 115 aa, Artificial Sequence
  Synthetic Polypeptide
EVQLVESGGGLVQPGGSLRLSCAASGFTFSNYGMNWIRQAPGKGLEWIGMIYYDSSEKHYADSVKGRFTISRDNSKNTLYLQMNSLRAEDTAVYYCAKGTTPDYWGQGTMVTVSS

SEQ ID NO: 26 - protein, 112 aa, Artificial Sequence
  Synthetic Polypeptide
DVVLTQSPLSLPVTLGQPASISCRSSQSLESSDGDTFLEWFQQRPGQSPRLLIYDVSTRFSGVPDRFSGSGSGTDFTLKISRVEAEDVGVYYCFQVTHDPVTFGQGTKLEIK

SEQ ID NO: 27 - protein, 115 aa, Artificial Sequence; identical to SEQ ID NO: 25
  Synthetic Polypeptide

SEQ ID NO: 28 - protein, 112 aa, Artificial Sequence
  Synthetic Polypeptide
DVVLTQSPLSLPVTLGQPASISCRSSQSLEESDGYTFLHWFQQRPGQSPRLLIYEVSTRFSGVPDRFSGSGSGTDFTLKISRVEAEDVGVYYCFQATHDPLTFGQGTKLEIK

SEQ ID NO: 29 - protein, 115 aa, Artificial Sequence
  Synthetic Polypeptide
EVQLVESGGGVVQPGRSLRLSCAASGFTFSNYGMNWVRQAPGKGLEWVAMIYYDSSEKHYADSVKGRFTISRDNSKNTLYLQMNSLRAEDTAVYYCARGTTPDYWGQGTMVTVSS

SEQ ID NO: 30 - protein, 112 aa, Artificial Sequence
  Synthetic Polypeptide
DVVLTQSPLSLPVTLGQPASISCRSSQSLADSDGDTFLHWFQQRPGQSPRLLIYAVSHRFSGVPDRFSGSGSGTDFTLKISRVEAEDVGVYYCFQATHDPVTFGQGTKLEIK

SEQ ID NO: 31 - protein, 115 aa, Artificial Sequence
  Synthetic Polypeptide
EVQLVESGGGLVQPGGSLRLSCAASGFTFSNYGMNWVRQAPGKGLEWVSMIYYDSSEKHYADSVKGRFTISRDNSKNTLYLQMNSLRAEDTAVYYCAKGTTPDYWGQGTMVTVSS

SEQ ID NO: 32 - protein, 112 aa, Artificial Sequence
  Synthetic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLSFGQGTKLEIK

SEQ ID NO: 33 - protein, 115 aa, Artificial Sequence; identical to SEQ ID NO: 25
  Synthetic Polypeptide

SEQ ID NO: 34 - protein, 113 aa, Artificial Sequence
  Synthetic Polypeptide
DVVLTQSPLSLPVTLGQPASISCRSSQSLEYSDGYTFLEWFQQRPGQSPRLLIYEVSNRFSGVPDRFSGSGSGTDFTLKISRVEAEDVGVYYCFQATHDPLTFGQGTKLEIKR

SEQ ID NO: 35 - protein, 116 aa, Artificial Sequence
  Synthetic Polypeptide
EVQLQQSGAELVRSGASVKLSCTASGFNIRDFYIHWVKQRPEQGLEWLGWIDPENGDIEYAPKFQGKATMTADTSSNTAYLQLNSLTSEDTALYYCNGNGYYLDYWGQGTTLTVSS

SEQ ID NO: 36 - protein, 113 aa, Artificial Sequence
  Synthetic Polypeptide
DVVMTQTPLTLSVTIGQPASISCKSGQSLLHSDGKTYLNWLLQRPGQSPKRLIYLVSKLDSGVPDRFTGSGSGTDFTLKISRVEAEDLGVYYCWQGTHSPWTFGGGTKLEIKR